(19) United States
(12) Patent Application Publication
(10) Pub. No.: US 2024/0277664 A1
(43) Pub. Date: Aug. 22, 2024

(54) COMPOSITIONS AND METHODS FOR TREATMENT OF SEXUAL DYSFUNCTION AND RELATED DISEASES, DISORDERS, AND CONDITIONS

(71) Applicant: Adamo Bioscience, Inc., Fernandina Beach, FL (US)

(72) Inventor: Molly Maloof, Fernandina Beach, FL (US)

(21) Appl. No.: 18/565,082

(22) PCT Filed: May 27, 2022
(86) PCT No.: PCT/US2022/031437

§ 371 Date: Nov. 28, 2023

Related U.S. Application Data
(60) Provisional application No. 63/194,740, filed on May 28, 2021.
(60) Provisional application No. 63/301,300, filed on Jan. 20, 2022.

(51) Int. Cl. A61K 31/4045; A61K 31/137; A61K 31/343; A61K 31/4458; A61P 15/12

(52) U.S. Cl. CPC A61K 31/4045; A61K 31/137; A61K 31/343; A61K 31/4458; A61P 15/12

(57) ABSTRACT

Provided herein are compositions and methods for the treatment of sexual dysfunction and related diseases, disorders, and conditions. An aspect of the present disclosure is a combination comprising at least two compounds independently selected from empathogens, stimulants, and tryptamines. In particular, the combinations disclosed relate to the empathogen (or entactogen) 1-(benzofuran-5-yl)-N-methylpropan-2-amine (5-MAPB) combined a tryptamine, such as 4-Acetoxy-N-methyl-N-allyltryptamine (4-AcO-MALT) or 4-Acetoxy-N-methyl-N-ethyltryptamine (metacetin or 4-AcO-MET), and a stimulant, such as 4-fluoromethylphenidate (4F-MPH), 2-fluoromethamphetamine (2-FMA), or 4-fluoromethamphetamine (4-FMA). The combinations disclosed herein can be utilized to augment and support sexual intimacy between mammals, particularly humans. In addition to augmenting sexual intimacy, the combinations disclosure herein can be used to treat sexual disorders.

### CROSS-REFERENCE

This application claims the benefit of U.S. Provisional Application No. 63/194,740 filed May 28, 2021; and U.S. Provisional Application No. 63/301,300 filed Jan. 20, 2022, each of which are incorporated herein by reference in their entirety.

### BACKGROUND OF THE INVENTION

Sexual dysfunction is a complex biopsychosocial problem that can alter human relationships, which have massive influence on health outcomes throughout the lifespan. It's likely that promoting love and social connection through synthetic chemicals could both improve health, deepen relationships and potentially prevent divorce. Furthermore, healing the detrimental effects of trauma on their relationships and sexual function could enable victims of trauma to resume a normal life.

Sexual disorders can result in severely diminished quality of life. Despite the fact that sexual disorders affect a greater percentage of women than men, there are 24 drugs approved for improving men's sexual health and only 2 drugs approved for women's sexual health. However, currently there is no FDA approved solutions for improving human relationships, sexual dysfunction and related disease, disorder, and conditions.

Accordingly, there is a need for new therapeutics and/or nutriceuticals for the treatment or alleviation of sexual disorders, especially for sexual disorders affecting women, and related diseases, disorders, and conditions, including, but are not limited to, sexual dysfunction, trauma-related conditions, attachment related conditions, social stress related conditions, psychosomatic diseases, personality disorders, mood disorders, eating disorders, addiction, grief, and physical, emotional, and sexual abuse.

### SUMMARY OF THE INVENTION

In one aspect, provided herein is a combination comprising:
a. one or more compounds of Formula (I)

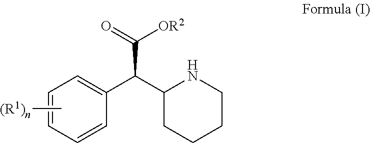

or pharmaceutically acceptable salts, solvates, or hydrates thereof, wherein
each R¹ is independently selected from hydrogen, halo, C₁₋₆alkyl, C₁₋₆haloalkyl, C₁₋₆alkoxy, C₁₋₆ haloalkoxy, —OR³, —C(O)R³, and —C(O)OR³;
R² is selected from hydrogen and C₁₋₆alkyl;
R³ is selected from hydrogen, C₁₋₆alkyl, C₃₋₆cycloalkyl, and C₆₋₁₀aryl; and
n is selected from 1, 2, 3, 4, and 5; and
b. one or more compounds of Formula (II)

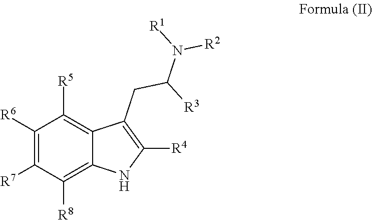

or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂;
R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

In another aspect, provided herein is a combination comprising:
a. one or more compounds of Formula (IV)

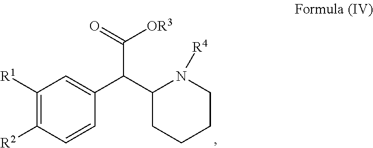

or pharmaceutically acceptable salts, solvates, or hydrates thereof, wherein:
R¹ and R² are independently selected from hydrogen, halo, C₁₋₆alkyl, C₁₋₆haloalkyl, C₁₋₆alkoxy, C₁₋₆haloalkoxy, —OR⁵, —C(O)R⁵, and —C(O)OR⁵; or R¹ and R² together with the atoms to which they are attached join to form 5-8 membered cycloalkyl, 5-8 membered heterocycloalkyl, or 6-membered aryl;
R³ is selected from hydrogen and C₁₋₆alkyl;
R⁴ is selected from hydrogen, C₁₋₆alkyl, and —C(O)OR⁵; and
R⁵ is selected from hydrogen and C₁₋₆alkyl, wherein C₁₋₆alkyl is optionally substituted with aryl, substituted aryl, heteroaryl, and substituted heteroaryl; and
b. one or more compounds of Formula (V)

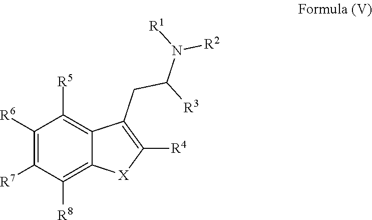

or pharmaceutically acceptable salts, solvates, or hydrates thereof, wherein
X is O or NH;
R¹ and R² are independently selected from C₁₋₆alkyl and C₂₋₆alkenyl, wherein the C₁₋₆alkyl and C₂₋₆alkenyl are optionally substituted with C₆₋₁₀aryl C₃₋₆cycloalkyl, or C₃₋₆heterocycloalkyl; or R¹ and R² together with the nitrogen to which they are attached join to form a 3-6 membered heterocycloalkyl;
R³ is selected from hydrogen, halo, C₁₋₆alkyl, C₁₋₆haloalkyl, C₁₋₆alkoxy, C₁₋₆haloalkoxy, and —N(R⁹)₂;
R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, —C(O)OR⁹ and —OP(O)(OH)₂; and
R⁹ is selected from hydrogen, C₁₋₆alkyl, C₁₋₆haloalkyl C₃₋₆cycloalkyl, and C₆₋₁₀aryl.

In some embodiments, the combination comprises about 1 mg to about 20 mg of said one or more compounds of Formula (I) or pharmaceutically acceptable salts, solvates, or hydrates thereof.

In some embodiments, the combination comprises about 1 mg to about 25 mg of said one or more compounds of Formula (II) or pharmaceutically acceptable salts, solvates, or hydrates thereof.

In some embodiments, the combination comprises about 1 mg to about 20 mg of said one or more compounds of Formula (IV) or pharmaceutically acceptable salts, solvates, or hydrates thereof.

In some embodiments, the combination comprises about 1 mg to about 25 mg of said one or more compounds of Formula (V) or pharmaceutically acceptable salts, solvates, or hydrates thereof.

In some embodiments, the combination further comprises one or more compounds of Formula (III)

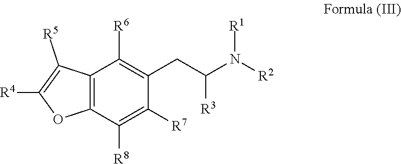

or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂;
R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

In some embodiments, the combination further comprises one or more compounds of Formula (VI)

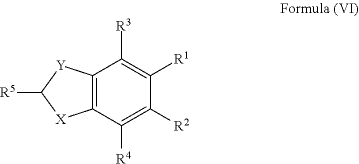

or pharmaceutically acceptable salts, solvates, or hydrates thereof, wherein
X and Y are independently selected from O and CH₂;
R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶;
R³ and R⁴ are independently selected from hydrogen, halogen, C₁₋₆alkyl, C₁₋₆haloalkyl, C₁₋₆-alkoxy, C₁₋₆haloalkoxy, —N(R⁶)₂, —OR⁶, —OC(O)R⁶, —C(O)R⁶, and —C(O)OR⁶;
R⁵ is selected from hydrogen, halogen, —N(R⁶)₂, and —OR⁶; and
R⁶ is selected from hydrogen, C₁₋₆alkyl, C₁₋₆haloalkyl C₃₋₆cycloalkyl, and C₆₋₁₀aryl.

In some embodiments, the combination further comprises one or more compounds of Formula (VII)

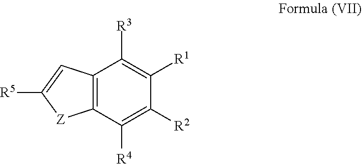

or pharmaceutically acceptable salts, solvates, or hydrates thereof, wherein
Z is selected from NH or O;
R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶;
R³ and R⁴ are independently selected from hydrogen, halogen, C₁₋₆alkyl, C₁₋₆haloalkyl, C₁₋₆-alkoxy, C₁₋₆haloalkoxy, —N(R⁶)₂, —OR⁶, —OC(O)R⁶, —C(O)R⁶, and —C(O)OR⁶;
R⁵ is selected from hydrogen, halogen, —N(R⁶)₂, and —OR⁶; and
R⁶ is selected from hydrogen, C₁₋₆alkyl, C₁₋₆haloalkyl C₃₋₆cycloalkyl, and C₆₋₁₀aryl.

In some embodiments, the combination further comprises one or more compounds

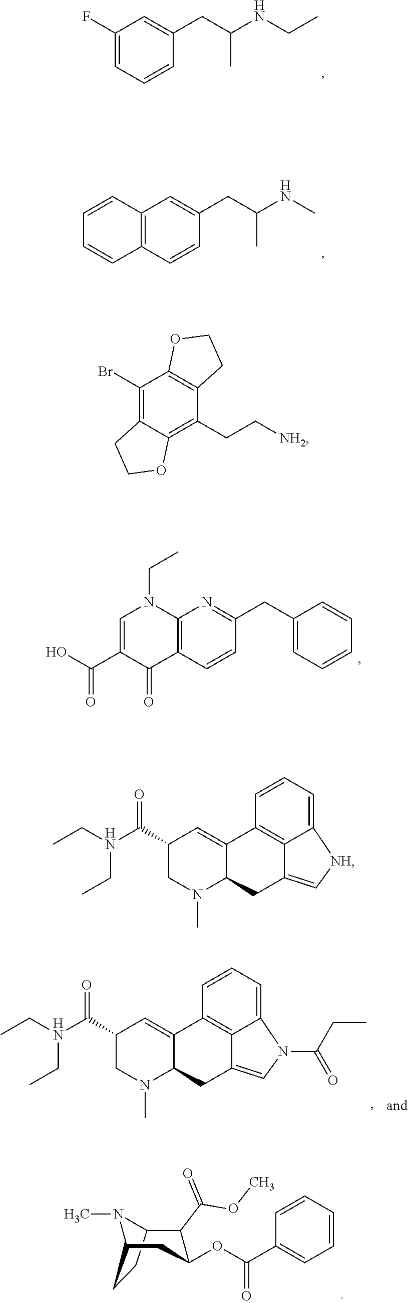

In some embodiments, the combination comprises about 1 mg to about 75 mg of said one or more compounds of Formula (III) or pharmaceutically acceptable salts, solvates, or hydrates thereof.

In some embodiments, the combination comprises about 1 mg to about 70 mg of said one or more compounds of Formula (VI) or pharmaceutically acceptable salts, solvates, or hydrates thereof.

In some embodiments, the combination comprises about 1 mg to about 70 mg of said one or more compounds of Formula (VII) or pharmaceutically acceptable salts, solvates, or hydrates thereof.

In some embodiments, said one or more compounds of Formula (I) comprises one or more compounds of Formula (I-A)

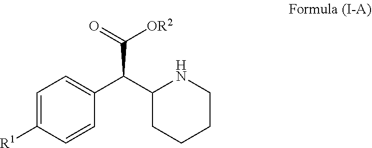

or pharmaceutically acceptable salts, solvates, or hydrates thereof, wherein
each R¹ is independently selected from hydrogen, halo, C₁₋₆alkyl, C₁₋₆haloalkyl, C₁₋₆alkoxy, C₁₋₆ haloalkoxy, —OR³, —C(O)R³, and —C(O)OR³;
R² is selected from hydrogen and C₁₋₆alkyl;
R³ is selected from hydrogen, C₁₋₆alkyl, C₃₋₆cycloalkyl, and C₆₋₁₀aryl; and
n is selected from 1, 2, 3, 4, and 5.

In some embodiments, said one or more compounds of Formula (II) comprises one or more compounds of Formula (II-A)

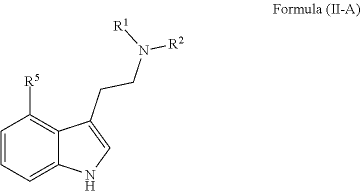

or pharmaceutically acceptable salts, solvates, or hydrates thereof, wherein
R¹ and R² are independently selected from C₁₋₆alkyl and C₂₋₆alkenyl;
R⁵ is selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

In some embodiments, said one or more compounds of Formula (III) comprises one or more compounds of Formula (III-A)

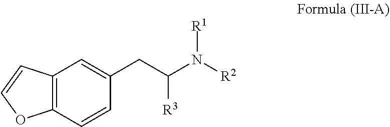

or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂; and
R⁹ is selected from hydrogen, C₁₋₆alkyl, C₁₋₆haloalkyl C₃₋₆cycloalkyl, and C₆₋₁₀aryl.

In some embodiments, said one or more compounds of Formula (I) comprise Compound

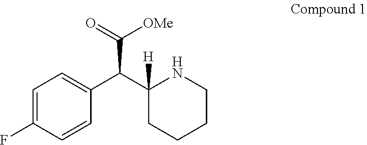 [SMILES: COC(=O)[C@H](c1ccc(F)cc1)[C@H]1CCCCN1]

or a pharmaceutically acceptable salt, solvate, or hydrate thereof.

In some embodiments, said one or more compounds of Formula (II) comprise Compound 2

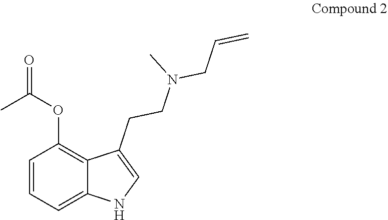 [SMILES: C=CCN(C)CCc1c[nH]c2cccc(OC(C)=O)c12]

or a pharmaceutically acceptable salt, solvate, or hydrate thereof.

In some embodiments, said one or more compounds of Formula (III) comprise Compound 3

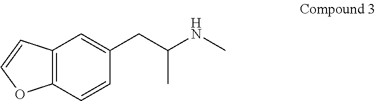

or a pharmaceutically acceptable salt, solvate, or hydrate thereof.

In another aspect, provided herein is a combination comprising one or more compounds selected from:

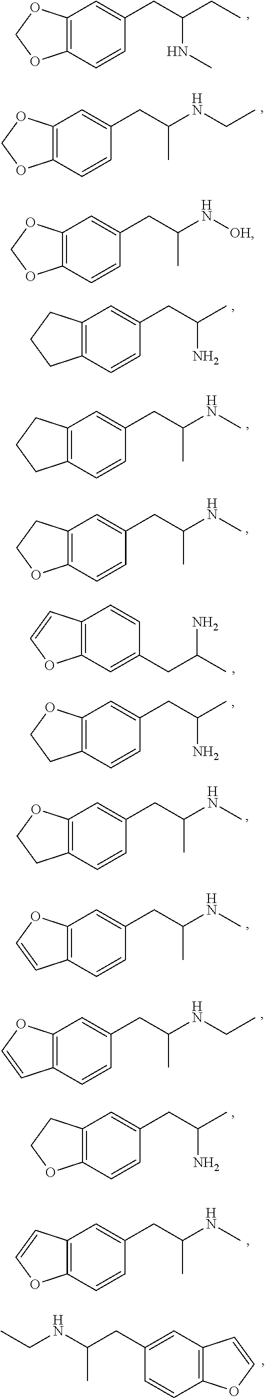

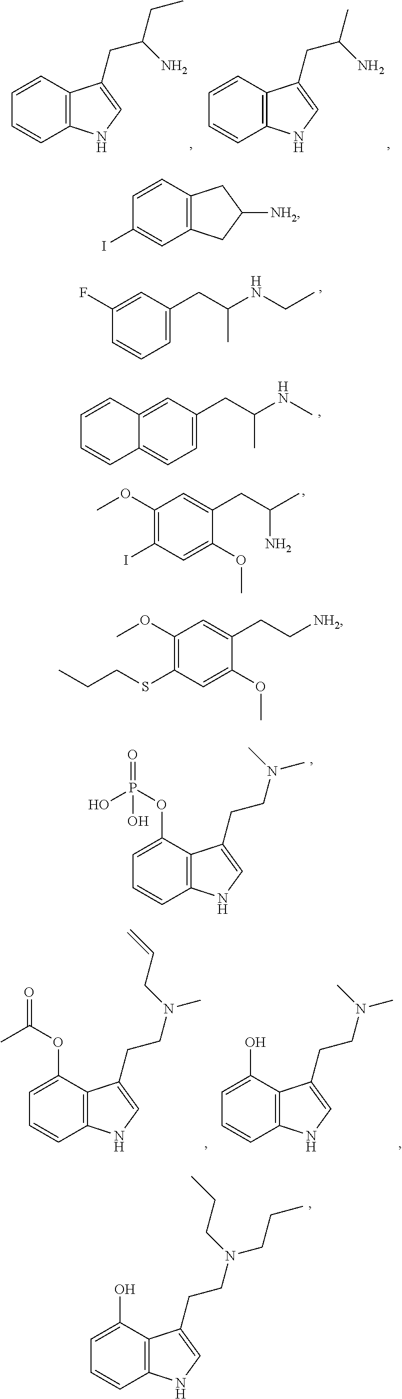

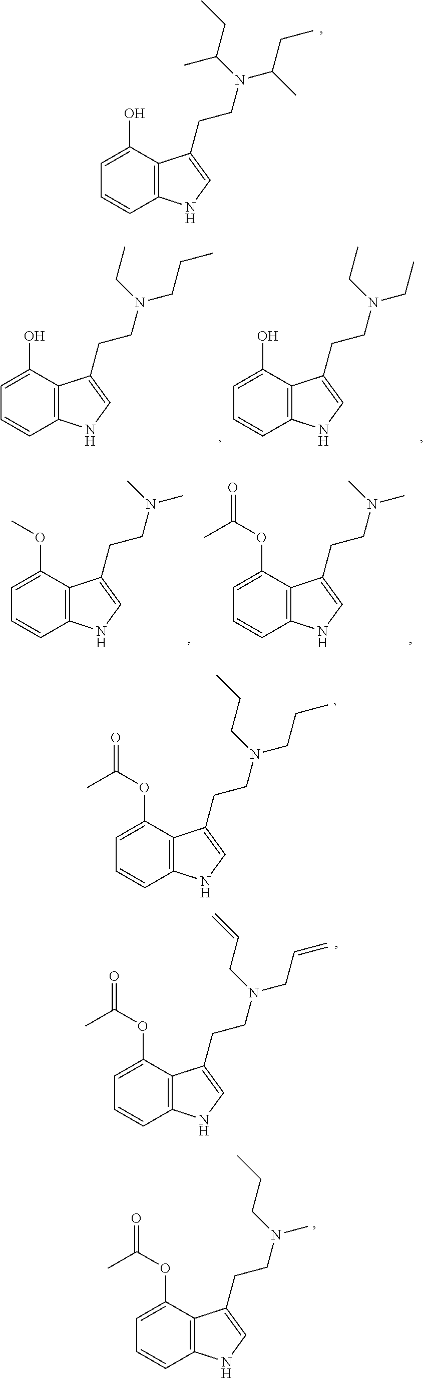

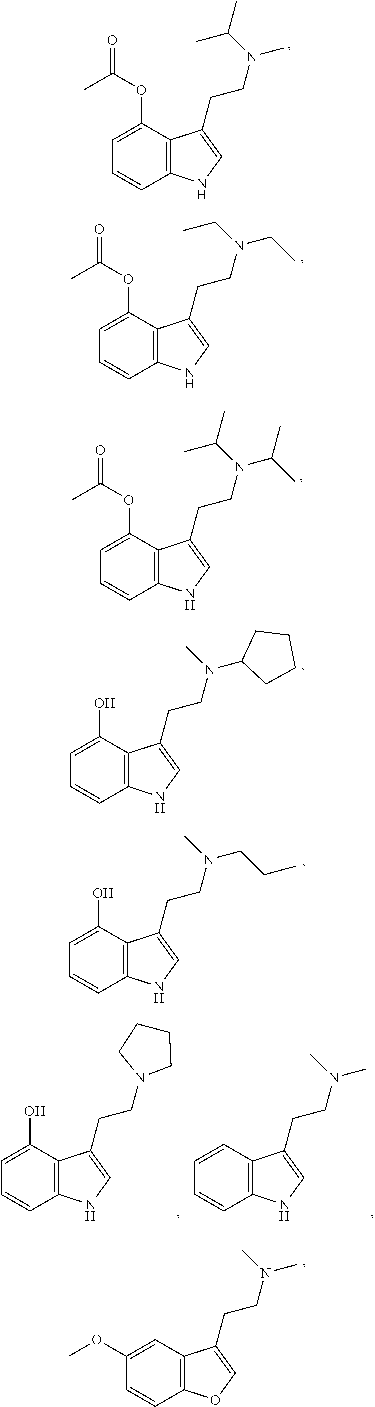

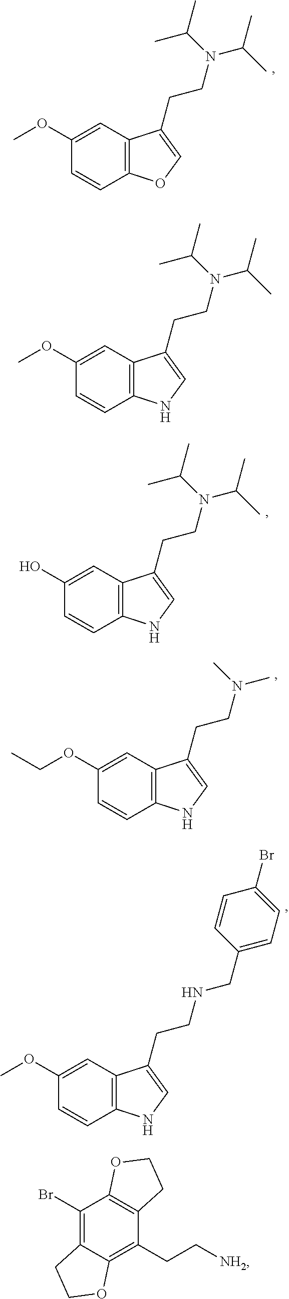

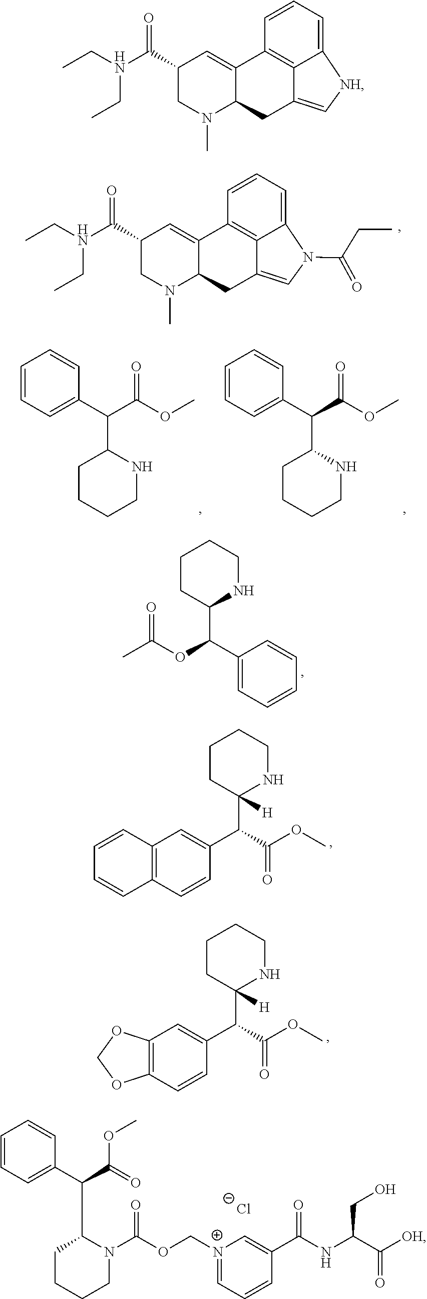

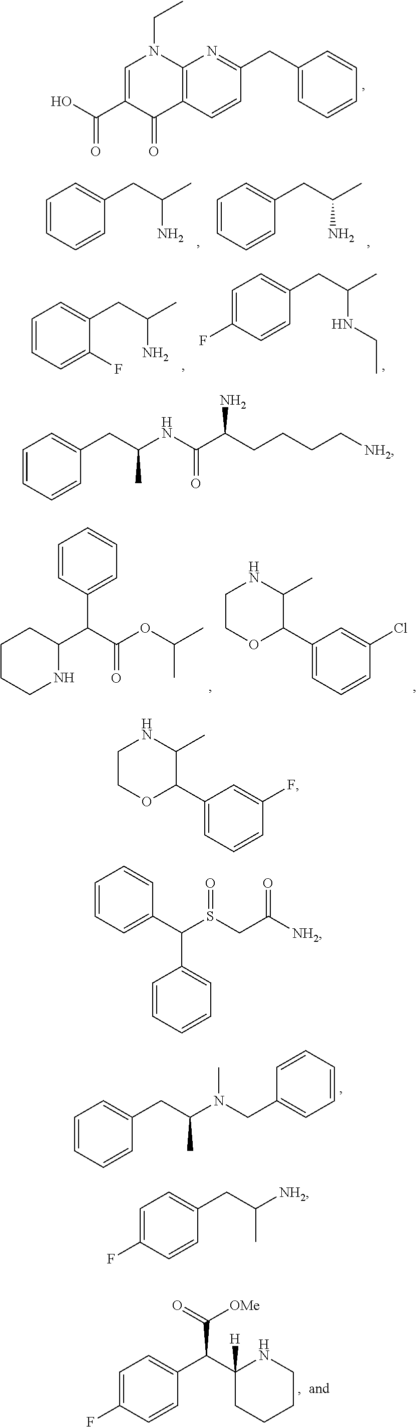

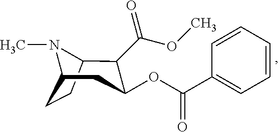

or pharmaceutically acceptable salts, solvates, or hydrates thereof.

In another aspect, provided herein is a pharmaceutical composition, comprising (A) a combination provided herein, or pharmaceutically acceptable salts, solvates, or hydrates thereof, and (B) a pharmaceutically acceptable carrier.

In another aspect, provided herein is an oral unit dosage form comprising (A) a combination or pharmaceutical composition provided herein, or a pharmaceutically acceptable salts, solvates, or hydrates thereof, and (B) a pharmaceutically acceptable carrier.

In some embodiments, the oral unit dosage form is a tablet. In some embodiments, the oral unit dosage form is a liquid.

In another aspect, provided herein is a method for treating or alleviating a sexual disorder in a subject in need thereof, the method comprising administering to said subject a combination provided herein, or pharmaceutically acceptable salts, solvates, or hydrates thereof, a pharmaceutical composition provided herein, or an oral unit dosage form provided herein. In some embodiments, said sexual disorder is selected from hypoactive sexual desire disorder, sexual arousal disorder, anorgasmia, vaginismus, dyspareunia, erectile dysfunction, diminished libido, and abnormal ejaculation.

In another aspect, provided herein is a method for treating or alleviating a disease, a disorder, or a condition in a subject in need thereof, the method comprising administering to said subject a combination provided herein, or pharmaceutically acceptable salts, solvates, or hydrates thereof, a pharmaceutical composition provided, or an oral unit dosage form provided herein. In some embodiments, the disease, the disorder, or the condition is selected from sexual abuse, mood disorders, phobia disorders, dissociative disorders, stress disorders, personality disorders, obsessive compulsive disorders, Tourette's disorder, grief, autism, eating disorders, alcohol use disorder, conduct disorder, conversion disorder, and addiction.

In another aspect, provided herein is a combination comprising two or more compounds, or pharmaceutically acceptable salts, solvates, or hydrates thereof, useful for the treatment of one or more sexual disorders, wherein said two or more compounds comprise an optionally fused benzene ring. In some embodiments, said combination comprises three or more compounds useful for the treatment of one or more sexual disorders, wherein said three or more compounds comprise an optionally fused benzene ring.

In some embodiments, said compounds are independently selected from phenethylamines, tryptamines, benzofurans, and amphetamines.

In some embodiments, at least one of said two or more compounds comprises Compound 1

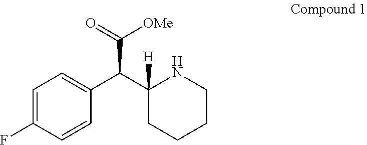 [SMILES: COC(=O)[C@H](c1ccc(F)cc1)[C@H]1CCCCN1]

or a pharmaceutically acceptable salt, solvate, or hydrate thereof.

In some embodiments, at least one of said two or more compounds comprises Compound 2

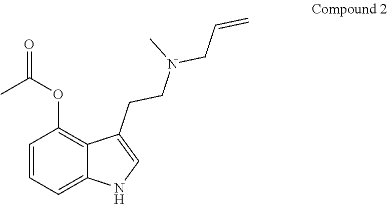 [SMILES: C=CCN(C)CCc1c[nH]c2cccc(OC(C)=O)c12]

or a pharmaceutically acceptable salt, solvate, or hydrate thereof.

In some embodiments, at least one of said two or more compounds comprises Compound 3

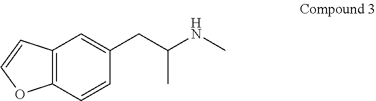

or a pharmaceutically acceptable salt, solvate, or hydrate thereof.

In some embodiments, Compound 1

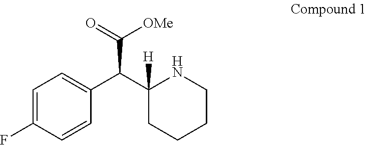 [SMILES: COC(=O)[C@H](c1ccc(F)cc1)[C@H]1CCCCN1]

or a pharmaceutically acceptable salt, solvate, or hydrate thereof, is present in the combination at about 1 mg to about 20 mg.

In some embodiments, Compound 2

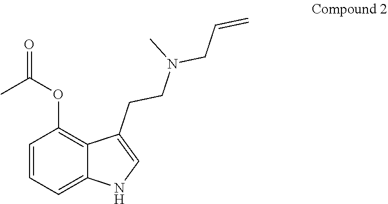 [SMILES: C=CCN(C)CCc1c[nH]c2cccc(OC(C)=O)c12]

or a pharmaceutically acceptable salt, solvate, or hydrate thereof, is present in the combination at about 1 mg to about 25 mg.

In some embodiments, Compound 3

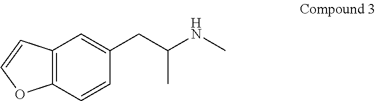

or a pharmaceutically acceptable salt, solvate, or hydrate thereof, is present in the combination at about 1 mg to about 70 mg.

In another aspect, provided herein is a pharmaceutical composition, comprising (A) a combination provided herein, or pharmaceutically acceptable salts, solvates, or hydrates thereof, and (B) a pharmaceutically acceptable carrier.

In another aspect, provided herein is an oral unit dosage form comprising (A) a combination or pharmaceutical composition provided herein, or a pharmaceutically acceptable salts, solvates, or hydrates thereof, and (B) a pharmaceutically acceptable carrier.

In some embodiments, the oral unit dosage form is a tablet. In some embodiments, the oral unit dosage form is a liquid.

In another aspect, provided herein is a combination comprising at least two compounds independently selected from empathogens, stimulants, and tryptamines. In some embodiments, the combination comprises an empathogen, a stimulant, and a tryptamine.

In another aspect, provided herein is a method for treating or alleviating a sexual disorder in a subject in need thereof, the method comprising administering to said subject a combination provided herein, or pharmaceutically acceptable salts, solvates, or hydrates thereof, a pharmaceutical composition provided herein, or pharmaceutically acceptable salts, solvates, or hydrates thereof, or an oral unit dosage form provided herein.

In some embodiments, said sexual disorder is selected from hypoactive sexual desire disorder, sexual arousal disorder, anorgasmia, vaginismus, dyspareunia, erectile dysfunction, diminished libido, and abnormal ejaculation.

In another aspect, provided herein is a method for enhancing sexual drive in a subject, the method comprising administering to said subject a combination provided herein, or pharmaceutically acceptable salts, solvates, or hydrates thereof, a pharmaceutical composition provided herein, or pharmaceutically acceptable salts, solvates, or hydrates thereof, or an oral unit dosage form provided herein.

Another aspect of the present disclosure is a combination comprising: one or more compounds of Formula (I)

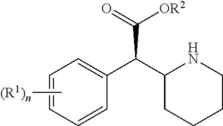

Formula (I) or pharmaceutically acceptable salts, solvates, or hydrates thereof, wherein each R¹ is independently selected from hydrogen, halo, C₁₋₆alkyl, C₁₋₆haloalkyl, C₁₋₆alkoxy, C₁₋₆haloalkoxy, —OR³, —C(O)R³, and —C(O)OR³; R² is selected from hydrogen and C₁₋₆alkyl; R³ is selected from hydrogen, C₁₋₆alkyl, C₃₋₆cycloalkyl, and C₆₋₁₀aryl; and n is selected from 1, 2, 3, 4, and 5; one or more compounds of Formula (II)

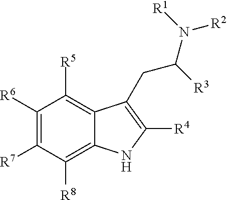

Formula (II) or pharmaceutically acceptable salts, solvates, or hydrates thereof, wherein R¹ and R² are independently selected from C₁₋₆alkyl and C₂₋₆alkenyl; R³ is selected from hydrogen, halo, C₁₋₆alkyl, C₁₋₆haloalkyl, C₁₋₆alkoxy, C₁₋₆haloalkoxy, and —N(R⁹)₂; R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and R⁹ is selected from hydrogen, C₁₋₆alkyl, C₁₋₆haloalkyl C₃₋₆cycloalkyl, and C₆₋₁₀aryl; and one or more compounds of Formula (III)

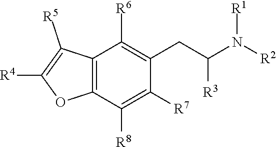

Formula (III) or pharmaceutically acceptable salts, solvates, or hydrates thereof, wherein R¹ and R² are independently selected from C₁₋₆alkyl and C₂₋₆alkenyl; R³ is selected from hydrogen, halo, C₁₋₆alkyl, C₁₋₆haloalkyl, C₁₋₆-alkoxy, C₁₋₆haloalkoxy, and —N(R⁹)₂; R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and R⁹ is selected from hydrogen, C₁₋₆alkyl, C₁₋₆haloalkyl C₃₋₆ cycloalkyl, and C₆₋₁₀aryl. In some embodiments, the combination comprises about 1 mg to about 20 mg of the one or more compounds of Formula (I) or pharmaceutically acceptable salts, solvates, or hydrates thereof. In some embodiments, the combination comprises about 1 mg to about 20 mg of the one or more compounds of Formula (II) or pharmaceutically acceptable salts, solvates, or hydrates thereof. In some embodiments, the combination comprises about 1 mg to about 140 mg of the one or more compounds of Formula (III) or pharmaceutically acceptable salts, solvates, or hydrates thereof. In some embodiments, the one or more compounds of Formula (III) are present at an amount that is 2-fold, 3-fold, 4-fold, 5-fold, 6-fold, 7-fold, or 8-fold greater than the amount of the one or more compounds of Formula (I), the one or more compounds of Formula (II), or both. In some embodiments, the one or more compounds of Formula (III) are present at an amount that is 7-fold greater than the amount of the one or more compounds of Formula (I) and the one or more compounds of Formula (II). In some embodiments, the combination comprises: 10 mg of the one or more compounds of Formula (I); 10 mg of the one or more compounds of Formula (II); and 70 mg of the one or more compounds of Formula (III). In some embodiments, the one or more compounds of Formula (I) is Compound 1. In some embodiments, the one or more compounds of Formula (II) is Compound 2. In some embodiments, the one or more compounds of Formula (III) is Compound 3.

Another aspect of the present disclosure is method for treating or alleviating a disease, a disorder, or a condition in a subject in need thereof, the method comprising administering to the subject: the combination of any one of the afore-mentioned embodiments, or pharmaceutically acceptable salts, solvates, or hydrates thereof. In some embodiments, the disease, the disorder, or the condition is selected from sexual abuse, mood disorders, phobia disorders, dissociative disorders, stress disorders, personality disorders, obsessive compulsive disorders, tourette's disorder, grief, autism, eating disorders, alcohol use disorder, conduct disorder, conversion disorder, and addiction. In some embodiments, the sexual abuse is selected from adult sexual disorder and child sexual disorder. In some embodiments, the mood disorder is selected from persistent depressive disorder or dysthymia, major depressive disorder, cyclothymic disorder, premenstrual dysphoric disorder, generalized anxiety disorder, and panic disorder. In some embodiments, the phobia disorder is selected from phobia of animal, phobia of blood-injection-injury, phobia of blood, phobia of injections or transfusions, phobia of injury, phobia of medical care, phobia of natural environment, and situational phobias. In some embodiments, the stress disorder is selected from acute stress disorder, posttraumatic stress disorder, unspecified trauma- and stressor-related disorder, nightmare disorder, adjustment disorder, reactive attachment disorder, and disinhibited attached disorder. In some embodiments, the personality disorder is selected from antisocial behavior, antisocial personality disorder, avoidant personality disorder, borderline personality disorder, narcissistic personality disorder, and obsessive-compulsive personality disorder. In some embodiments, the eating disorder is selected from avoidant/restrictive food intake disorder, binge-eating disorder, body dysmorphic disorder, bulimia nervosa, and anorexia nervosa. In some embodiments, the addiction is selected from tobacco use addiction, opioid use disorder, and alcohol use disorder.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE DRAWINGS

The novel features of the present disclosure are set forth with particularity in the appended claims. An understanding of the features and advantages of the present disclosure may be obtained by reference to the following detailed description that sets forth illustrative embodiments, in which the principles of the present disclosure are utilized, and the accompanying drawings of which:

FIG. 1 shows a bar graph of the positive feelings that were experienced by the participants.

FIG. 2 shows a bar graph of the negative feelings that were experienced by the participants.

FIG. 3 shows the survey results on the questions “would the participant like to participate in said experience again”.

FIG. 4 shows the survey results on the question “how soon would the participate like to participate in said experience again”

FIG. 5 shows the rating of the overall level of satisfaction (on a scale of 0-100) of the participants from this experiment.

FIG. 6 shows survey results on how likely (on a scale of 0-100) the participants would recommend this experience to a friend or loved one.

FIG. 7 shows an exemplary combination of the disclosure.

### DETAILED DESCRIPTION OF THE INVENTION

The present disclosure relates to combinations and methods useful for the treatment of sexual disorders and related disease, disorder, and condition. Specifically, the present disclosure a novel fixed-dosed combination drug that combines a benzofuran, a stimulant, and a psychedelic synergistically to reproduce the neurobiology of love, which can be used as a treatment for a variety of medical disorders, such as sexual dysfunction, abuse, a mood disorder, a phobia disorder, a dissociative disorder, a stress disorder, a personality disorders, an obsessive-compulsive disorders Tourette's disorder, grief, autism, an eating disorder, an alcohol use disorder, a conduct disorder, a conversion disorder, an addiction, or other related disease, disorder, and conditions as described herein.

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. An alkyl group is optionally substituted by one or more substituents such as those substituents described herein.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. An alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. An alkynyl group is optionally substituted by one or more substituents such as those substituents described herein.

“Halo” or “Halogen”, alone or in combination with any other term means halogens such as chloro (Cl), fluoro (F), bromo (Br) and iodo (I).

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

The term “alkoxy” refers to the group R—O—, where R is optionally substituted alkyl or optionally substituted cycloalkyl, or optionally substituted alkenyl or optionally substituted alkynyl; where alkyl, alkenyl, alkynyl, and cycloalkyl are as defined herein. The term “haloalkoxy” refers to an alkoxy group substituted with one or more halogens. Representative examples of alkoxy groups include but are not limited to methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “cycloalkyl” refers to unless otherwise mentioned, carbocyclic groups of from 3 to 6 carbon atoms having a single cyclic ring or multiple condensed rings or spirocyclic rings or bridged rings. This definition encompasses rings that are saturated or partially unsaturated. Such cycloalkyl groups include, by way of example, single ring structures such as cyclopropyl, cyclobutyl, cyclopentyl, cyclopentenyl, cyclohexyl, cyclohexenyl, and the like.

The term “aryl” refers to a radical derived from a hydrocarbon ring system comprising hydrogen, 6 to 30 carbon atoms and at least one aromatic ring. This definition encompasses monocyclic, bicyclic, tricyclic or tetracyclic ring system, as well as fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

“Pharmaceutically acceptable salt” embraces salts with a pharmaceutically acceptable acid or base. Pharmaceutically acceptable acids include both inorganic acids, for example hydrochloric, sulfuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulfonic, ethanesulfonic, benzenesulfonic or p-toluenesulfonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines. In some embodiments, the compound is a pharmaceutically acceptable salt derived from acids including, but not limited to, the following: acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, or p-toluenesulfonic acid.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition comprising a compound of the present disclosure and a pharmaceutically acceptable carrier.

“Carrier” refers to a diluent, adjuvant, excipient, or vehicle with which the therapeutic is administered. In some embodiments, such pharmaceutical carriers are sterile liquids, such as water and oils, including those of petroleum, animal, vegetable or synthetic origin, including but not limited to peanut oil, soybean oil, mineral oil, sesame oil and the like. In some embodiments, water is a carrier when the pharmaceutical composition is administered orally. In some embodiments, saline and aqueous dextrose are exemplary carriers when the pharmaceutical composition is administered intravenously. In some embodiments, saline solutions and aqueous dextrose and glycerol solutions are employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some embodiments, the composition comprises minor amounts of wetting or emulsifying agents, or pH buffering agents. In some embodiments, these compositions take the form of solutions, suspensions, emulsions, tablets, pills, capsules, powders, sustained-release formulations and the like. In some embodiments, the composition is formulated as a suppository, with traditional binders and carriers such as triglycerides. In some embodiments, an oral formulation comprises carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, for example in purified form, together with a suitable amount of carrier so as to provide the form for proper administration to the patient. The formulation should suit the mode of administration.

“Combined” or “in combination” or“combination” should be understood as a functional coadministration, encompassing scenarios wherein compounds are administered separately, in different formulations, different modes of administration (for example subcutaneous, intravenous or oral) and different times of administration. In some embodiments, the individual compounds of such combinations are administered sequentially in separate pharmaceutical compositions. In some embodiments, the individual compounds of such combinations are administered simultaneously in combined pharmaceutical compositions.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “sexual disorder” encompasses problems that affect a subject's ability to engage in and derive satisfaction from sexual activity. Sexual disorders include, but are not limited to, disorders that affect a subject's desire to engage in sexual activity, disorders that affect a subject's ability to experience physical sexual arousal, disorders that affect a subject's ability to achieve orgasm, and disorders that cause pain during sexual activity. Examples of sexual disorders include, but are not limited to, hypoactive sexual desire disorder, sexual arousal disorder, anorgasmia, vaginismus, dyspareunia, erectile dysfunction, diminished libido, and abnormal ejaculation. As used herein, “sexual disorder” encompasses sexual problems regardless of cause. Common causes of sexual disorders include, but are not limited to, stress, mental illness, side effects from medications (such as selective serotonin reuptake inhibitors), past sexual trauma, intimate relationship problems, hormonal imbalances, diabetes, heart disease, neurological disorders, chronic illness, alcoholism, and drug abuse.

As used herein, “hypoactive sexual disorder” or “diminished libido” refers to a level of sexual desire (sex drive) and/or romantic love that is sufficiently low to cause distress or interpersonal difficulties to a subject.

As used herein, “sexual arousal disorder” refers to an inability to achieve or maintain a typical physiological response to sexual arousal. In subjects with vaginas, symptoms include, but are not limited to, lack of vaginal lubrication, lack of vaginal dilation or lengthening, decreased genital tumescence, and decreased genital sensation. In subjects with penises, symptoms include, but are not limited to, erectile dysfunction.

As used herein, “anorgasmia” refers to an inability to achieve orgasm despite a level of sexual stimulation that would typically result in orgasm. Anorgasmia is far more common in subjects with vaginas than in subjects with penises and is especially common in post-menopausal subjects. Anorgasmia can be chronic or situational and is a leading cause of sexual frustration.

As used herein, “vaginismus” refers to a condition in which involuntary muscle spasms make vaginal penetration difficult or impossible. Vaginismus can result in problems with sexual activity (inability to achieve digital, penile, or object penetration), gynecological health (inability to accommodate gynecological instruments such as speculums), and personal hygiene (inability to insert menstrual products such as tampons or menstrual cups).

As used herein, “dyspareunia” refers to pain during sexual intercourse. In subjects with vaginas, causes of dyspareunia include, but are not limited to, vaginal lesions, retroversion of the uterus, urinary tract infection, lack of vaginal lubrication, scar tissue, abnormal growths, tender pelvic sites, infections, cancer of the reproductive tract, anatomical variants, hormonal causes, pelvic masses, bladder irritation, vulvodynia, muscular dysfunction, and vaginismus. In subjects with penises, causes of dyspareunia include, but are not limited to, infections, interstitial cystitis, sexually transmitted diseases, anatomic deformities of the penis (such as Peyronie's disease), frenulum breve, and phimosis.

As used herein, “erectile dysfunction” refers to the inability to achieve or maintain an erection of sufficient quality to enjoy sexual activity. In some instances, a subject experiencing erectile dysfunction may be able to attain a low-quality erection that is insufficiently rigid to achieve penetration. Causes of erectile dysfunction include, but are not limited to, cardiovascular disease, diabetes, neurological problems, side effects of drugs, and psychological problems.

As used herein, “abnormal ejaculation” refers to a condition in which a subject with a penis experiences ejaculation in a timeframe that causes distress or interpersonal problems to the subject. “Delayed ejaculation” refers to an ability to ejaculate in a timely fashion despite sufficient sexual stimulation; delayed ejaculation is a form of anorgasmia. Subjects experiencing delayed ejaculation are often able to ejaculate from masturbation but are unable to ejaculate with a sexual partner, leading to sexual frustration. “Premature ejaculation” refers to a condition in which a subject with a penis experiences orgasm soon after the onset of sexual activity and with minimal sexual stimulation. Due to the refractory period, premature ejaculation often results in the cessation of penile penetration and can lead to sexual frustration.

As used herein, the terms “subject with a penis” and “subject with a vagina” are meant to reflect a subject's anatomy and not gender identity. For example, “subject with a penis” is meant to encompass all subjects with penises, including, but not limited to, cis men (those assigned male at birth who identify as male), trans women (those assigned male at birth who identify as female), and non-binary individuals (those who do not identify on the gender binary). Likewise, “subject with a vagina” is meant to encompass all subjects with vaginas, including, but not limited to, cis women (those assigned female at birth who identify as female), trans men (those assigned female at birth who identify as male), and non-binary individuals (those who do not identify on the gender binary). Similarly, the terms “men” and “women” are meant to indicate gender identity regardless of anatomy (i.e., “men” encompasses both cis and trans men, and “women” encompasses both cis and trans women).

As used herein, “phenethylamine” refers to a compound containing a substituted core corresponding to the following formula:

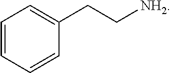

In other words, “phenethylamines” includes phenethylamine itself as well as all compounds wherein one or more hydrogen atoms of phenethylamine are replaced with substituents. Examples of phenethylamines include, but are not limited to, dopamine and epinephrine.

As used herein, “benzofuran” refers to a compound containing a substituted core corresponding to the following formula:

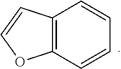

In other words, “benzofurans” includes benzofuran itself as well as all compounds wherein one or more hydrogen atoms of benzofuran are replaced with substituents. Examples of benzofurans include, but are not limited to, BPAP and brofaromine.

As used herein, “amphetamine” refers to a compound containing a substituted core corresponding to the following formula:

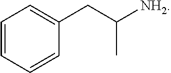

In other words, “amphetamines” includes amphetamine itself as well as all compounds wherein one or more hydrogen atoms of amphetamine are replaced with substituents. Amphetamines are a sub-class of phenethylamines. Examples of amphetamines include, but are not limited to, methamphetamine and ephedrine.

As used herein, “tryptamine” refers to a compound containing a substituted core corresponding to the following formula:

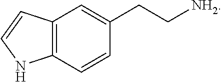 [SMILES: NCCc1ccc2[nH]ccc2c1]

In other words, “tryptamines” includes tryptamine itself as well as all compounds wherein one or more hydrogen atoms of amphetamine are replaced with substituents. Tryptamines are a sub-class of phenethylamines. Examples of tryptamines include, but are not limited to, serotonin and DMT.

As used herein, “stimulant” refers to a substance that increases activity of the central nervous system. Stimulants belong to a wide variety of chemical classes, including but not limited to phenethylamines and amphetamines. Examples of stimulants include, but are not limited to, amphetamine, caffeine, ephedrine, MDMA, nicotine, and cocaine.

As used herein, “empathogen” or “entactogen” refers to a substance that produces feelings of emotional communion, oneness, relatedness, emotional openness, empathy, or sympathy in a subject. Most empathogens are phenethylamines, amphetamines, or tryptamines. Examples of empathogens include, but are not limited to, MDMA, MDA, MDEA, MDOH, MBDB, 6-APB, methylone, mephedrone, aMT, aET, and MDAI.

### Compounds and Combinations

In one aspect, provided herein is a combination comprising:
a. one or more compounds of Formula (I)

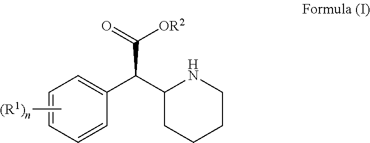

or pharmaceutically acceptable salts, solvates, or hydrates thereof, wherein
each R¹ is independently selected from hydrogen, halo, C₁₋₆alkyl, C₁₋₆haloalkyl, C₁₋₆alkoxy, C₁₋₆haloalkoxy, —OR³, —C(O)R³, and —C(O)OR³;
R² is selected from hydrogen and C₁₋₆alkyl;
R³ is selected from hydrogen, C₁₋₆alkyl, C₃₋₆cycloalkyl, and C₆₋₁₀aryl; and
n is selected from 1, 2, 3, 4, and 5; and
b. one or more compounds of Formula (II)

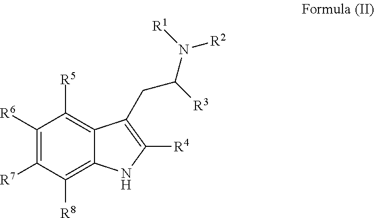

or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂;
R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

In some embodiments, for a compound of Formula (I), each R¹ is independently selected from hydrogen, halo, C₁₋₆alkyl, C₁₋₆haloalkyl, C₁₋₆alkoxy, C₁₋₆haloalkoxy, —OR³, —C(O)R³, and —C(O)OR³. In some embodiments, for a compound of Formula (I), each R¹ is independently selected from hydrogen, halo, C₁₋₆alkyl, C₁₋₆alkoxy, —OR³, —C(O)R³, and —C(O)OR³. In some embodiments, for a compound of Formula (I), each R¹ is independently selected from hydrogen, halo, —OR³, —C(O)R³, and —C(O)OR³. In some embodiments, for a compound of Formula (I), each R¹ is independently selected from hydrogen, halo, and —OR³. In some embodiments, for a compound of Formula (I), each R¹ is independently selected from hydrogen and halo. In some embodiments, for a compound of Formula (I), each R¹ is halo. In some embodiments, for a compound of Formula (I), each R¹ is fluoro.

In some embodiments, for a compound of Formula (I), R² is selected from hydrogen and C₁₋₆alkyl. In some embodiments, for a compound of Formula (I), R² is hydrogen. In some embodiments, for a compound of Formula (I), R² is C₁₋₆alkyl. In some embodiments, for a compound of Formula (I), R² is methyl.

In some embodiments, for a compound of Formula (I), R³ is selected from hydrogen, C₁₋₆ alkyl, C₃₋₆cycloalkyl, and C₆₋₁₀aryl. In some embodiments, for a compound of Formula (I), R³ is selected from hydrogen and C₁₋₆alkyl. In some embodiments, for a compound of Formula (I), R³ is hydrogen. In some embodiments, for a compound of Formula (I), R³ is C₁₋₆alkyl. In some embodiments, for a compound of Formula (I), R³ is C₃₋₆cycloalkyl. In some embodiments, for a compound of Formula (I), R³ is C₆₋₁₀aryl.

In some embodiments, for a compound of Formula (I), n is selected from 1, 2, 3, 4, and 5. In some embodiments, for a compound of Formula (I), n is 1. In some embodiments, for a compound of Formula (I), n is 2. In some embodiments, for a compound of Formula (I), n is 3. In some embodiments, for a compound of Formula (I), n is 4. In some embodiments, for a compound of Formula (I), n is 5.

In some embodiments, for a compound of Formula (II), R¹ and R² are independently selected from C₁₋₆alkyl and C₂₋₆alkenyl. In some embodiments, for a compound of Formula (II), R¹ is C₁₋₆alkyl. In some embodiments, for a compound of Formula (II), R¹ is C₂₋₆alkenyl. In some embodiments, for a compound of Formula (II), R² is C₁₋₆alkyl. In some embodiments, for a compound of Formula (II), R² is C₂₋₆alkenyl. In some embodiments, for a compound of Formula (II), R¹ is C₁₋₆alkyl and R² is C₁₋₆alkyl. In some embodiments, for a compound of Formula (II), R¹ is C₂₋₆alkenyl and R² is C₁₋₆alkyl. In some embodiments, for a compound of Formula (II), R¹ is C₁₋₆alkyl and R² is C₂₋₆alkenyl. In some embodiments, for a compound of Formula (II), R¹ is C₂₋₆alkenyl and R² is C₂₋₆alkenyl. In some embodiments, for a compound of Formula (II), R¹ is methyl and R² is allyl. In some embodiments, for a compound of Formula (II), R¹ is methyl and R² is allyl. In some embodiments, for a compound of Formula (II), R¹ is methyl and R² is ethyl. In some embodiments, for a compound of Formula (II), R¹ is ethyl and R² is methyl. In some embodiments, for a compound of Formula (II), R¹ is methyl and R² is isopropyl. In some embodiments, for a compound of Formula (II), R¹ is isopropyl and R² is methyl. In some embodiments, for a compound of Formula (II), R¹ is isopropyl and R² is isopropyl. In some embodiments, for a compound of Formula (II), R¹ is methyl and R² is n-propyl. In some embodiments, for a compound of Formula (II), R¹ is n-propyl and R² is methyl.

In some embodiments, for a compound of Formula (II), R³ is selected from hydrogen, halo, C₁₋₆alkyl, C₁₋₆haloalkyl, C₁₋₆alkoxy, C₁₋₆haloalkoxy, and —N(R⁹)₂. In some embodiments, for a compound of Formula (II), R³ is selected from hydrogen, halo, C₁₋₆alkyl, and C₁₋₆haloalkyl. In some embodiments, for a compound of Formula (II), R³ is selected from hydrogen and C₁₋₆alkyl. In some embodiments, for a compound of Formula (II), R³ is hydrogen. In some embodiments, for a compound of Formula (II), R³ is C₁₋₆alkyl. In some embodiments, for a compound of Formula (II), R³ is methyl.

In some embodiments, for a compound of Formula (II), R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹. In some embodiments, for a compound of Formula (II), R⁴, R⁵, R⁶, R⁷, and R⁸ are independently selected from hydrogen, halo, C₁₋₆alkyl, C₁₋₆haloalkyl, C₁₋₆alkoxy, —OR⁹, —OC(O)R⁹, —C(O)R⁹, and —C(O)OR⁹. In some embodiments, for a compound of Formula (II), R⁴, R⁵, R⁶, R⁷, and R⁸ are independently selected from hydrogen, halo, C₁₋₆alkoxy, —OR⁹, and —OC(O)R⁹. In some embodiments, for a compound of Formula (II), R⁴, R⁵, R⁶, R⁷, and R⁸ are independently selected from hydrogen, C₁₋₆alkoxy, —OR⁹, and —OC(O)R⁹. In some embodiments, for a compound of Formula (II), R⁴ and R⁸ are hydrogen, and R⁵ and R⁶ are independently selected from hydrogen, C₁₋₆alkoxy, —OR⁹, and —OC(O)R⁹. In some embodiments, for a compound of Formula (II), R⁴, R⁶, and R⁸ are hydrogen, and R⁵ is —OH. In some embodiments, for a compound of Formula (II), R⁴, R⁶, and R⁸ are hydrogen, and R⁵ is —OC(O)Me. In some embodiments, for a compound of Formula (II), R⁴, R⁵, and R⁸ are hydrogen, and R⁶ is —OH. In some embodiments, for a compound of Formula (II), R⁴, R⁵, and R⁸ are hydrogen, and R⁶ is —OMe.

In some embodiments, for a compound of Formula (II), R⁹ is selected from hydrogen, C₁₋₆ alkyl, C₁₋₆haloalkyl C₃₋₆cycloalkyl, and C₆₋₁₀aryl. In some embodiments, for a compound of Formula (II), R⁹ is selected from hydrogen, C₁₋₆alkyl, and C₁₋₆haloalkyl. In some embodiments, for a compound of Formula (II), R⁹ is selected from hydrogen and C₁₋₆alkyl. In some embodiments, for a compound of Formula (II), R⁹ is hydrogen. In some embodiments, for a compound of Formula (II), R⁹ is C₁₋₆alkyl. In some embodiments, for a compound of Formula (II), R⁹ is methyl.

In some embodiments, the combination comprises about 1 mg to about 20 mg of said one or more compounds of Formula (I) or pharmaceutically acceptable salts, solvates, or hydrates thereof. In some embodiments, the combination comprises about 1 mg to about 10 mg of said one or more compounds of Formula (I) or pharmaceutically acceptable salts, solvates, or hydrates thereof. In some embodiments, the combination comprises about 10 mg to about 20 mg of said one or more compounds of Formula (I) or pharmaceutically acceptable salts, solvates, or hydrates thereof. In some embodiments, the combination comprises about 1 mg to about 5 mg of said one or more compounds of Formula (I) or pharmaceutically acceptable salts, solvates, or hydrates thereof. In some embodiments, the combination comprises about 5 mg to about 10 mg of said one or more compounds of Formula (I) or pharmaceutically acceptable salts, solvates, or hydrates thereof. In some embodiments, the combination comprises about 10 mg to about 15 mg of said one or more compounds of Formula (I) or pharmaceutically acceptable salts, solvates, or hydrates thereof. In some embodiments, the combination comprises about 15 mg to about 20 mg of said one or more compounds of Formula (I) or pharmaceutically acceptable salts, solvates, or hydrates thereof. In some embodiments, the combination comprises about 20 mg to about 25 mg of said one or more compounds of Formula (I) or pharmaceutically acceptable salts, solvates, or hydrates thereof. In some embodiments, the combination comprises about 1 mg of said one or more compounds of Formula (I) or pharmaceutically acceptable salts, solvates, or hydrates thereof. In some embodiments, the combination comprises about 2 mg of said one or more compounds of Formula (I) or pharmaceutically acceptable salts, solvates, or hydrates thereof. In some embodiments, the combination comprises about 3 mg of said one or more compounds of Formula (I) or pharmaceutically acceptable salts, solvates, or hydrates thereof. In some embodiments, the combination comprises about 4 mg of said one or more compounds of Formula (I) or pharmaceutically acceptable salts, solvates, or hydrates thereof. In some embodiments, the combination comprises about 5 mg of said one or more compounds of Formula (I) or pharmaceutically acceptable salts, solvates, or hydrates thereof. In some embodiments, the combination comprises about 6 mg of said one or more compounds of Formula (I) or pharmaceutically acceptable salts, solvates, or hydrates thereof. In some embodiments, the combination comprises about 7 mg of said one or more compounds of Formula (I) or pharmaceutically acceptable salts, solvates, or hydrates thereof. In some embodiments, the combination comprises about 8 mg of said one or more compounds of Formula (I) or pharmaceutically acceptable salts, solvates, or hydrates thereof. In some embodiments, the combination comprises about 9 mg of said one or more compounds of Formula (I) or pharmaceutically acceptable salts, solvates, or hydrates thereof. In some embodiments, the combination comprises about 10 mg of said one or more compounds of Formula (I) or pharmaceutically acceptable salts, solvates, or hydrates thereof. In some embodiments, the combination comprises about 11 mg of said one or more compounds of Formula (I) or pharmaceutically acceptable salts, solvates, or hydrates thereof. In some embodiments, the combination comprises about 12 mg of said one or more compounds of Formula (I) or pharmaceutically acceptable salts, solvates, or hydrates thereof. In some embodiments, the combination comprises about 13 mg of said one or more compounds of Formula (I) or pharmaceutically acceptable salts, solvates, or hydrates thereof. In some embodiments, the combination comprises about 14 mg of said one or more compounds of Formula (I) or pharmaceutically acceptable salts, solvates, or hydrates thereof. In some embodiments, the combination comprises about 15 mg of said one or more compounds of Formula (I) or pharmaceutically acceptable salts, solvates, or hydrates thereof. In some embodiments, the combination comprises about 16 mg of said one or more compounds of Formula (I) or pharmaceutically acceptable salts, solvates, or hydrates thereof. In some embodiments, the combination comprises about 17 mg of said one or more compounds of Formula (I) or pharmaceutically acceptable salts, solvates, or hydrates thereof. In some embodiments, the combination comprises about 18 mg of said one or more compounds of Formula (I) or pharmaceutically acceptable salts, solvates, or hydrates thereof. In some embodiments, the combination comprises about 19 mg of said one or more compounds of Formula (I) or pharmaceutically acceptable salts, solvates, or hydrates thereof. In some embodiments, the combination comprises about 20 mg of said one or more compounds of Formula (I) or pharmaceutically acceptable salts, solvates, or hydrates thereof. In some embodiments, the combination comprises about 25 mg of said one or more compounds of Formula (I) or pharmaceutically acceptable salts, solvates, or hydrates thereof.

In some embodiments, the combination comprises about 1 mg to about 25 mg of said one or more compounds of Formula (II) or pharmaceutically acceptable salts, solvates, or hydrates thereof. In some embodiments, the combination comprises about 1 mg to about 10 mg of said one or more compounds of Formula (II) or pharmaceutically acceptable salts, solvates, or hydrates thereof. In some embodiments, the combination comprises about 10 mg to about 25 mg of said one or more compounds of Formula (II) or pharmaceutically acceptable salts, solvates, or hydrates thereof. In some embodiments, the combination comprises about 1 mg to about 5 mg of said one or more compounds of Formula (II) or pharmaceutically acceptable salts, solvates, or hydrates thereof. In some embodiments, the combination comprises about 5 mg to about 10 mg of said one or more compounds of Formula (II) or pharmaceutically acceptable salts, solvates, or hydrates thereof. In some embodiments, the combination comprises about 10 mg to about 15 mg of said one or more compounds of Formula (II) or pharmaceutically acceptable salts, solvates, or hydrates thereof. In some embodiments, the combination comprises about 15 mg to about 20 mg of said one or more compounds of Formula (II) or pharmaceutically acceptable salts, solvates, or hydrates thereof. In some embodiments, the combination comprises about 20 mg to about 25 mg of said one or more compounds of Formula (II) or pharmaceutically acceptable salts, solvates, or hydrates thereof. In some embodiments, the combination comprises about 1 mg of said one or more compounds of Formula (II) or pharmaceutically acceptable salts, solvates, or hydrates thereof. In some embodiments, the combination comprises about 2 mg of said one or more compounds of Formula (II) or pharmaceutically acceptable salts, solvates, or hydrates thereof. In some embodiments, the combination comprises about 3 mg of said one or more compounds of Formula (II) or pharmaceutically acceptable salts, solvates, or hydrates thereof. In some embodiments, the combination comprises about 4 mg of said one or more compounds of Formula (II) or pharmaceutically acceptable salts, solvates, or hydrates thereof. In some embodiments, the combination comprises about 5 mg of said one or more compounds of Formula (II) or pharmaceutically acceptable salts, solvates, or hydrates thereof. In some embodiments, the combination comprises about 6 mg of said one or more compounds of Formula (II) or pharmaceutically acceptable salts, solvates, or hydrates thereof. In some embodiments, the combination comprises about 7 mg of said one or more compounds of Formula (II) or pharmaceutically acceptable salts, solvates, or hydrates thereof. In some embodiments, the combination comprises about 8 mg of said one or more compounds of Formula (II) or pharmaceutically acceptable salts, solvates, or hydrates thereof. In some embodiments, the combination comprises about 9 mg of said one or more compounds of Formula (II) or pharmaceutically acceptable salts, solvates, or hydrates thereof. In some embodiments, the combination comprises about 10 mg of said one or more compounds of Formula (II) or pharmaceutically acceptable salts, solvates, or hydrates thereof. In some embodiments, the combination comprises about 11 mg of said one or more compounds of Formula (II) or pharmaceutically acceptable salts, solvates, or hydrates thereof. In some embodiments, the combination comprises about 12 mg of said one or more compounds of Formula (II) or pharmaceutically acceptable salts, solvates, or hydrates thereof. In some embodiments, the combination comprises about 13 mg of said one or more compounds of Formula (II) or pharmaceutically acceptable salts, solvates, or hydrates thereof. In some embodiments, the combination comprises about 14 mg of said one or more compounds of Formula (II) or pharmaceutically acceptable salts, solvates, or hydrates thereof. In some embodiments, the combination comprises about 15 mg of said one or more compounds of Formula (II) or pharmaceutically acceptable salts, solvates, or hydrates thereof. In some embodiments, the combination comprises about 16 mg of said one or more compounds of Formula (II) or pharmaceutically acceptable salts, solvates, or hydrates thereof. In some embodiments, the combination comprises about 17 mg of said one or more compounds of Formula (II) or pharmaceutically acceptable salts, solvates, or hydrates thereof. In some embodiments, the combination comprises about 18 mg of said one or more compounds of Formula (II) or pharmaceutically acceptable salts, solvates, or hydrates thereof. In some embodiments, the combination comprises about 19 mg of said one or more compounds of Formula (II) or pharmaceutically acceptable salts, solvates, or hydrates thereof. In some embodiments, the combination comprises about 20 mg of said one or more compounds of Formula (II) or pharmaceutically acceptable salts, solvates, or hydrates thereof. In some embodiments, the combination comprises about 21 mg of said one or more compounds of Formula (II) or pharmaceutically acceptable salts, solvates, or hydrates thereof. In some embodiments, the combination comprises about 22 mg of said one or more compounds of Formula (II) or pharmaceutically acceptable salts, solvates, or hydrates thereof. In some embodiments, the combination comprises about 23 mg of said one or more compounds of Formula (II) or pharmaceutically acceptable salts, solvates, or hydrates thereof. In some embodiments, the combination comprises about 24 mg of said one or more compounds of Formula (II) or pharmaceutically acceptable salts, solvates, or hydrates thereof. In some embodiments, the combination comprises about 25 mg of said one or more compounds of Formula (II) or pharmaceutically acceptable salts, solvates, or hydrates thereof.

In some embodiments, the compound of Formula (I) is selected from

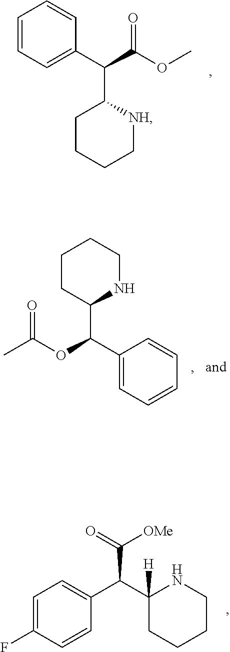

or pharmaceutically acceptable salts, solvates, or hydrates thereof.

In some embodiments the compound of Formula (II) is selected from

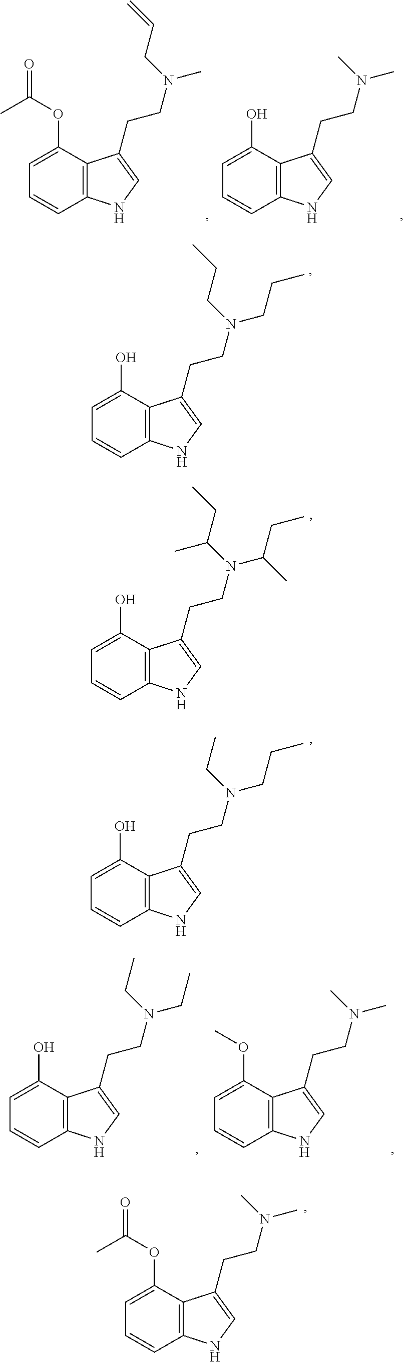

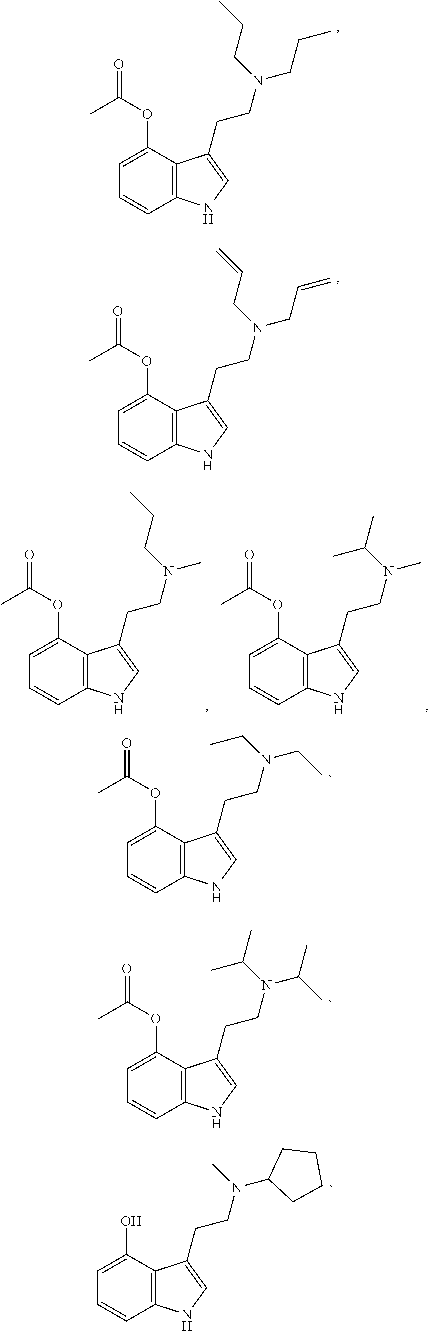

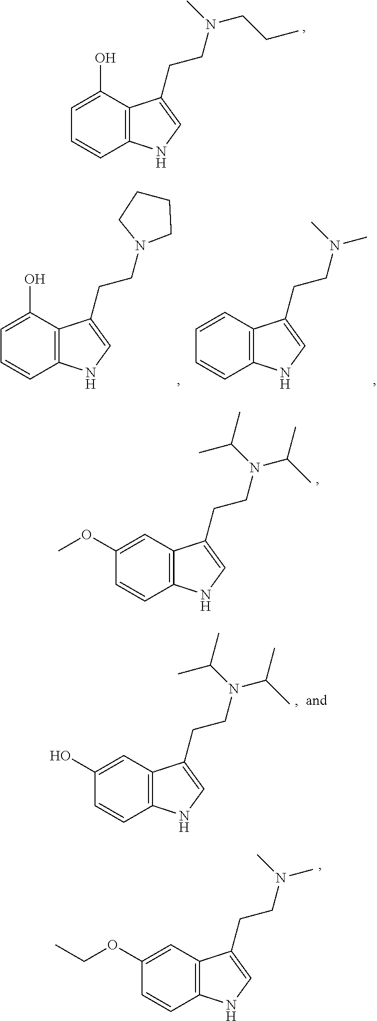

or pharmaceutically acceptable salts, solvates, or hydrates thereof.

In a second aspect, provided herein is a combination comprising:
a. one or more compounds of Formula (IV):

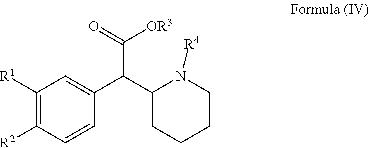

or a pharmaceutically acceptable salts, solvates, or hydrates thereof, wherein:
R¹ and R² are independently selected from hydrogen, halo, C₁₋₆alkyl, C₁₋₆haloalkyl, C₁₋₆alkoxy, C₁₋₆haloalkoxy, —OR⁵, —C(O)R⁵, and —C(O)OR⁵; or R¹ and R² together with the atoms to which they are attached join to form 5-8 membered cycloalkyl, 5-8 membered heterocycloalkyl, or 6-membered aryl;
R³ is selected from hydrogen and C1-6alkyl;
R⁴ is selected from hydrogen, C₁₋₆alkyl, and —C(O)OR⁵; and
R⁵ is selected from hydrogen and C₁₋₆alkyl, wherein C₁₋₆alkyl is optionally substituted with aryl, substituted aryl, heteroaryl, and substituted heteroaryl; and
b. one or more compounds of Formula (V)

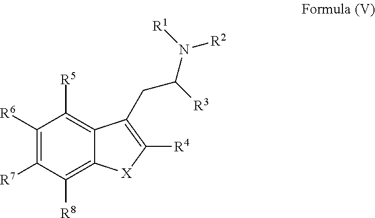

or pharmaceutically acceptable salts, solvates, or hydrates thereof, wherein
X is O or NH;
R¹ and R² are independently selected from C₁₋₆alkyl and C₂₋₆alkenyl, wherein the C₁₋₆ alkyl and C₂₋₆alkenyl are optionally substituted with C₆₋₁₀aryl C₃₋₆cycloalkyl, or C₃₋₆heterocycloalkyl; or R¹ and R² together with the nitrogen to which they are attached join to form a 3-6 membered heterocycloalkyl;
R³ is selected from hydrogen, halo, C₁₋₆alkyl, C₁₋₆haloalkyl, C₁₋₆alkoxy, C₁₋₆-haloalkoxy, and —N(R⁹)₂;
R⁴, R⁵, R⁶, R⁷, and R⁸ are independently selected from hydrogen, halo, C₁₋₆alkyl, C₁₋₆ haloalkyl, C₁₋₆alkoxy, C₁₋₆haloalkoxy, —N(R⁹)₂, —OR⁹, —OC(O)R⁹, —C(O)R⁹, —C(O)OR⁹ and —OP(O)OH₂; and
R⁹ is selected from hydrogen, C₁₋₆alkyl, C₁₋₆haloalkyl C₃₋₆cycloalkyl, and C₆₋₁₀aryl.

In some embodiments of the compound of Formula (IV), X is O. In some embodiments of the compound of Formula (IV), X is NH.

In some embodiments of the compound of Formula (IV), each R¹ and R² are independently selected from hydrogen, halo, C₁₋₆alkyl, C₁₋₆haloalkyl, C₁₋₆alkoxy, C₁₋₆haloalkoxy, —OR³, —C(O)R³, and —C(O)OR³. In some embodiments of the compound of Formula (IV), each R¹ and R² are independently selected from hydrogen, halo, C₁₋₆alkyl, C₁₋₆alkoxy, —OR³, —C(O)R³, and —C(O)OR³. In some embodiments of the compound of Formula (IV), each R¹ and R² are independently selected from hydrogen, halo, —OR³, —C(O)R³, and —C(O)OR³. In some embodiments of the compound of Formula (IV), each R¹ and R² are independently selected from hydrogen, halo, and —OR³. In some embodiments of the compound of Formula (IV), each R¹ and R² are is independently selected from hydrogen and halo. In some embodiments of the compound of Formula (IV), each R¹ and R² are halo. In some embodiments of the compound of Formula (IV), each R¹ and R² are fluoro.

In some embodiments of the compound of Formula (IV), R¹ and R² together with the atoms to which they are attached join to form a 5-8 membered cycloalkyl, 5-8 membered heterocycloakyl, or a 6-membered aryl. In some embodiments of the compound of Formula (IV), R¹ and R² together with the atoms to which they are attached join to form a 5-8 membered cycloalkyl. In some embodiments of the compound of Formula (IV), R¹ and R² together with the atoms to which they are attached join to form a 55-8 membered heterocycloakyl. In some embodiments of the compound of Formula (IV), R¹ and R² together with the atoms to which they are attached join to form a 6-membered aryl.

In some embodiments of the compound of Formula (IV), R³ is selected from hydrogen and C₁₋₆alkyl. In some embodiments of the compound of Formula (IV), R³ is hydrogen. In some embodiments of the compound of Formula (IV), R³ is C₁₋₆alkyl. In some embodiments of the compound of Formula (IV), R³ is methyl.

In some embodiments of the compound of Formula (IV), R⁴ is selected from hydrogen, C₁₋₆alkyl, and —C(O)OR⁵. In some embodiments of the compound of Formula (IV), R⁴ is hydrogen. In some embodiments of the compound of Formula (IV), R⁴ is C₁₋₆alkyl. In some embodiments of the compound of Formula (IV), R⁴ is —C(O)OR⁵. In some embodiments of the compound of Formula (IV), R⁴ is methyl. In some embodiments of the compound of Formula (IV), R⁴ is

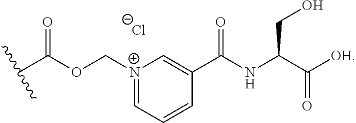 [SMILES: *C(=O)OC[n+]1cccc(C(=O)N[C@@H](CO)C(=O)O)c1 ; [Cl-]]

In some embodiments of the compound of Formula (IV), R⁵ is selected from hydrogen, C₁₋₆alkyl, C₃₋₆cycloalkyl, and C₆₋₁₀aryl, wherein the C₁₋₆alkyl, C₃₋₆cycloalkyl, and C₆₋₁₀aryl are optionally substituted with aryl, substituted aryl, heteroaryl, and substituted heteroaryl. In some embodiments of the compound of Formula (IV), R⁵ is selected from hydrogen and C₁₋₆alkyl, wherein the C₁₋₆alkyl is optionally substituted with aryl, substituted aryl, heteroaryl, and substituted heteroaryl. In some embodiments of the compound of Formula (IV), R⁵ is hydrogen. In some embodiments of the compound of Formula (IV), R⁵ is C₁₋₆alkyl, wherein the C₁₋₆alkyl is optionally substituted with aryl, substituted aryl, heteroaryl, and substituted heteroaryl.

In some embodiments of a compound of Formula (V), R¹ and R² are independently selected from C₁₋₆alkyl and C₂₋₆alkenyl, wherein the C₁₋₆alkyl and C₂₋₆alkenyl are optionally substituted with C₆₋₁₀aryl C₃₋₆cycloalkyl, or C₃₋₆heterocycloalkyl; or R¹ and R² together with the nitrogen to which they are attached join to form a 3-6 membered heterocycloalkyl. In some embodiments of a compound of Formula (V), R¹ is C₁₋₆alkyl. In some embodiments of a compound of Formula (V), R¹ is C₂₋₆alkenyl. In some embodiments of a compound of Formula (V), R² is C₁₋₆alkyl. In some embodiments of a compound of Formula (V), R² is C₂₋₆alkenyl. In some embodiments of a compound of Formula (V), R¹ is C₁₋₆alkyl and R² is C₁₋₆alkyl. In some embodiments of a compound of Formula (V), R¹ is C₂₋₆alkenyl and R² is C₁₋₆alkyl. In some embodiments of a compound of Formula (V), R¹ is C₁₋₆alkyl and R² is C₂₋₆alkenyl. In some embodiments of a compound of Formula (V), R¹ is C₂₋₆alkenyl and R² is C₂₋₆alkenyl. In some embodiments of a compound of Formula (V), R¹ is methyl and R² is allyl. In some embodiments of a compound of Formula (V), R¹ is methyl and R² is allyl. In some embodiments of a compound of Formula (V), R¹ is methyl and R² is ethyl. In some embodiments of a compound of Formula (V), R¹ is ethyl and R² is methyl. In some embodiments of a compound of Formula (V), R¹ is methyl and R² is isopropyl. In some embodiments of a compound of Formula (V) R¹ is isopropyl and R² is methyl. In some embodiments of a compound of Formula (V), R¹ is isopropyl and R² is isopropyl. In some embodiments of a compound of Formula (V), R¹ is methyl and R² is n-propyl. In some embodiments of a compound of Formula (V), R¹ is n-propyl and R² is methyl.

In some embodiments of a compound of Formula (V), R³ is selected from hydrogen, halo, C₁₋₆alkyl, C₁₋₆haloalkyl, C₁₋₆alkoxy, C₁₋₆haloalkoxy, and —N(R⁹)₂. In some embodiments of a compound of Formula (V), R³ is selected from hydrogen, halo, C₁₋₆alkyl, and C₁₋₆haloalkyl. In some embodiments of a compound of Formula (V), R³ is selected from hydrogen and C₁₋₆alkyl. In some embodiments of a compound of Formula (V), R³ is hydrogen. In some embodiments of a compound of Formula (V), R³ is C₁₋₆alkyl. In some embodiments of a compound of Formula (V), R³ is methyl.

In some embodiments of the compound of Formula (V), R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, —C(O)OR⁹, and —OP(O)(OH)₂. In some embodiments of the compound of Formula (V), R⁴, R⁵, R⁶, R⁷, and R⁸ are independently selected from hydrogen, halo, C₁₋₆alkyl, C₁₋₆haloalkyl, C₁₋₆alkoxy, —OR⁹, —OC(O)R⁹, —C(O)R⁹, —C(O)OR⁹, and —OP(O)(OH)₂. In some embodiments of the compound of Formula (V), R⁴, R⁵, R⁶, R⁷, and R⁸ are independently selected from hydrogen, halo, C₁₋₆alkoxy, —OR⁹, and —OC(O)R⁹. In some embodiments of the compound of Formula (V), R⁴, R⁵, R⁶, R⁷, and R⁸ are independently selected from hydrogen, C₁₋₆alkoxy, —OR⁹, and —OC(O)R⁹. In some embodiments of the compound of Formula (V), R⁴ and R⁸ are hydrogen, and R⁵ and R⁶ are independently selected from hydrogen, C₁₋₆alkoxy, —OR⁹, and —OC(O)R⁹. In some embodiments of the compound of Formula (V), R⁴, R⁶, and R⁸ are hydrogen, and R⁵ is —OH. In some embodiments of the compound of Formula (V), R⁴, R⁶, and R⁸ are hydrogen, and R⁵ is —OC(O)Me. In some embodiments of the compound of Formula (V), R⁴, R⁵, and R⁸ are hydrogen, and R⁶ is —OH. In some embodiments of the compound of Formula (V), R⁴, R⁵, and R⁸ are hydrogen, and R⁶ is —OMe.

In some embodiments of the compound of Formula (V), R⁹ is selected from hydrogen, C₁₋₆ alkyl, C₁₋₆haloalkyl C₃₋₆cycloalkyl, and C₆₋₁₀aryl. In some embodiments of the compound of Formula (V), R⁹ is selected from hydrogen, C₁₋₆alkyl, and C₁₋₆haloalkyl. In some embodiments of the compound of Formula (V), R⁹ is selected from hydrogen and C₁₋₆alkyl. In some embodiments of the compound of Formula (V), R⁹ is hydrogen. In some embodiments of the compound of Formula (V), R⁹ is C₁₋₆alkyl. In some embodiments of the compound of Formula (V), R⁹ is methyl.

In some embodiments, the combination comprises about 1 mg to about 20 mg of said one or more compounds of Formula (IV) or pharmaceutically acceptable salts, solvates, or hydrates thereof. In some embodiments, the combination comprises about 1 mg to about 10 mg of said one or more compounds of Formula (IV) or pharmaceutically acceptable salts, solvates, or hydrates thereof. In some embodiments, the combination comprises about 10 mg to about 20 mg of said one or more compounds of Formula (IV) or pharmaceutically acceptable salts, solvates, or hydrates thereof. In some embodiments, the combination comprises about 1 mg to about 5 mg of said one or more compounds of Formula (IV) or pharmaceutically acceptable salts, solvates, or hydrates thereof. In some embodiments, the combination comprises about 5 mg to about 10 mg of said one or more compounds of Formula (IV) or pharmaceutically acceptable salts, solvates, or hydrates thereof. In some embodiments, the combination comprises about 10 mg to about 15 mg of said one or more compounds of Formula (IV) or pharmaceutically acceptable salts, solvates, or hydrates thereof. In some embodiments, the combination comprises about 15 mg to about 20 mg of said one or more compounds of Formula (IV) or pharmaceutically acceptable salts, solvates, or hydrates thereof. In some embodiments, the combination comprises about 20 mg to about 25 mg of said one or more compounds of Formula (IV) or pharmaceutically acceptable salts, solvates, or hydrates thereof. In some embodiments, the combination comprises about 1 mg of said one or more compounds of Formula (IV) or pharmaceutically acceptable salts, solvates, or hydrates thereof. In some embodiments, the combination comprises about 2 mg of said one or more compounds of Formula (IV) or pharmaceutically acceptable salts, solvates, or hydrates thereof. In some embodiments, the combination comprises about 3 mg of said one or more compounds of Formula (IV) or pharmaceutically acceptable salts, solvates, or hydrates thereof. In some embodiments, the combination comprises about 4 mg of said one or more compounds of Formula (IV) or pharmaceutically acceptable salts, solvates, or hydrates thereof. In some embodiments, the combination comprises about 5 mg of said one or more compounds of Formula (IV) or pharmaceutically acceptable salts, solvates, or hydrates thereof. In some embodiments, the combination comprises about 6 mg of said one or more compounds of Formula (IV) or pharmaceutically acceptable salts, solvates, or hydrates thereof. In some embodiments, the combination comprises about 7 mg of said one or more compounds of Formula (IV) or pharmaceutically acceptable salts, solvates, or hydrates thereof. In some embodiments, the combination comprises about 8 mg of said one or more compounds of Formula (IV) or pharmaceutically acceptable salts, solvates, or hydrates thereof. In some embodiments, the combination comprises about 9 mg of said one or more compounds of Formula (IV) or pharmaceutically acceptable salts, solvates, or hydrates thereof. In some embodiments, the combination comprises about 10 mg of said one or more compounds of Formula (IV) or pharmaceutically acceptable salts, solvates, or hydrates thereof. In some embodiments, the combination comprises about 11 mg of said one or more compounds of Formula (IV) or pharmaceutically acceptable salts, solvates, or hydrates thereof. In some embodiments, the combination comprises about 12 mg of said one or more compounds of Formula (IV) or pharmaceutically acceptable salts, solvates, or hydrates thereof. In some embodiments, the combination comprises about 13 mg of said one or more compounds of Formula (IV) or pharmaceutically acceptable salts, solvates, or hydrates thereof. In some embodiments, the combination comprises about 14 mg of said one or more compounds of Formula (IV or pharmaceutically acceptable salts, solvates, or hydrates thereof. In some embodiments, the combination comprises about 15 mg of said one or more compounds of Formula (IV) or pharmaceutically acceptable salts, solvates, or hydrates thereof. In some embodiments, the combination comprises about 16 mg of said one or more compounds of Formula (IV) or pharmaceutically acceptable salts, solvates, or hydrates thereof. In some embodiments, the combination comprises about 17 mg of said one or more compounds of Formula (IV) or pharmaceutically acceptable salts, solvates, or hydrates thereof. In some embodiments, the combination comprises about 18 mg of said one or more compounds of Formula (IV) or pharmaceutically acceptable salts, solvates, or hydrates thereof. In some embodiments, the combination comprises about 19 mg of said one or more compounds of Formula (IV) or pharmaceutically acceptable salts, solvates, or hydrates thereof. In some embodiments, the combination comprises about 20 mg of said one or more compounds of Formula (IV) or pharmaceutically acceptable salts, solvates, or hydrates thereof. In some embodiments, the combination comprises about 25 mg of said one or more compounds of Formula (IV) or pharmaceutically acceptable salts, solvates, or hydrates thereof.

In some embodiments, the combination comprises about 1 mg to about 25 mg of said one or more compounds of Formula (V) or pharmaceutically acceptable salts, solvates, or hydrates thereof. In some embodiments, the combination comprises about 1 mg to about 10 mg of said one or more compounds of Formula (V) or pharmaceutically acceptable salts, solvates, or hydrates thereof. In some embodiments, the combination comprises about 10 mg to about 25 mg of said one or more compounds of Formula (V) or pharmaceutically acceptable salts, solvates, or hydrates thereof. In some embodiments, the combination comprises about 1 mg to about 5 mg of said one or more compounds of Formula (V) or pharmaceutically acceptable salts, solvates, or hydrates thereof. In some embodiments, the combination comprises about 5 mg to about 10 mg of said one or more compounds of Formula (V) or pharmaceutically acceptable salts, solvates, or hydrates thereof. In some embodiments, the combination comprises about 10 mg to about 15 mg of said one or more compounds of Formula (V) or pharmaceutically acceptable salts, solvates, or hydrates thereof. In some embodiments, the combination comprises about 15 mg to about 20 mg of said one or more compounds of Formula (V) or pharmaceutically acceptable salts, solvates, or hydrates thereof. In some embodiments, the combination comprises about 20 mg to about 25 mg of said one or more compounds of Formula (V) or pharmaceutically acceptable salts, solvates, or hydrates thereof. In some embodiments, the combination comprises about 1 mg of said one or more compounds of Formula (V) or pharmaceutically acceptable salts, solvates, or hydrates thereof. In some embodiments, the combination comprises about 2 mg of said one or more compounds of Formula (V) or pharmaceutically acceptable salts, solvates, or hydrates thereof. In some embodiments, the combination comprises about 3 mg of said one or more compounds of Formula (V) or pharmaceutically acceptable salts, solvates, or hydrates thereof. In some embodiments, the combination comprises about 4 mg of said one or more compounds of Formula (V) or pharmaceutically acceptable salts, solvates, or hydrates thereof. In some embodiments, the combination comprises about 5 mg of said one or more compounds of Formula (V) or pharmaceutically acceptable salts, solvates, or hydrates thereof. In some embodiments, the combination comprises about 6 mg of said one or more compounds of Formula (V) or pharmaceutically acceptable salts, solvates, or hydrates thereof. In some embodiments, the combination comprises about 7 mg of said one or more compounds of Formula (V) or pharmaceutically acceptable salts, solvates, or hydrates thereof. In some embodiments, the combination comprises about 8 mg of said one or more compounds of Formula (V) or pharmaceutically acceptable salts, solvates, or hydrates thereof. In some embodiments, the combination comprises about 9 mg of said one or more compounds of Formula (V) or pharmaceutically acceptable salts, solvates, or hydrates thereof. In some embodiments, the combination comprises about 10 mg of said one or more compounds of Formula (V) or pharmaceutically acceptable salts, solvates, or hydrates thereof. In some embodiments, the combination comprises about 11 mg of said one or more compounds of Formula (V) or pharmaceutically acceptable salts, solvates, or hydrates thereof. In some embodiments, the combination comprises about 12 mg of said one or more compounds of Formula (V) or pharmaceutically acceptable salts, solvates, or hydrates thereof. In some embodiments, the combination comprises about 13 mg of said one or more compounds of Formula (V) or pharmaceutically acceptable salts, solvates, or hydrates thereof. In some embodiments, the combination comprises about 14 mg of said one or more compounds of Formula (V) or pharmaceutically acceptable salts, solvates, or hydrates thereof. In some embodiments, the combination comprises about 15 mg of said one or more compounds of Formula (V) or pharmaceutically acceptable salts, solvates, or hydrates thereof. In some embodiments, the combination comprises about 16 mg of said one or more compounds of Formula (V) or pharmaceutically acceptable salts, solvates, or hydrates thereof. In some embodiments, the combination comprises about 17 mg of said one or more compounds of Formula (V) or pharmaceutically acceptable salts, solvates, or hydrates thereof. In some embodiments, the combination comprises about 18 mg of said one or more compounds of Formula (V) or pharmaceutically acceptable salts, solvates, or hydrates thereof. In some embodiments, the combination comprises about 19 mg of said one or more compounds of Formula (V) or pharmaceutically acceptable salts, solvates, or hydrates thereof. In some embodiments, the combination comprises about 20 mg of said one or more compounds of Formula (V) or pharmaceutically acceptable salts, solvates, or hydrates thereof. In some embodiments, the combination comprises about 21 mg of said one or more compounds of Formula (V) or pharmaceutically acceptable salts, solvates, or hydrates thereof. In some embodiments, the combination comprises about 22 mg of said one or more compounds of Formula (V) or pharmaceutically acceptable salts, solvates, or hydrates thereof. In some embodiments, the combination comprises about 23 mg of said one or more compounds of Formula (V) or pharmaceutically acceptable salts, solvates, or hydrates thereof. In some embodiments, the combination comprises about 24 mg of said one or more compounds of Formula (V) or pharmaceutically acceptable salts, solvates, or hydrates thereof. In some embodiments, the combination comprises about 25 mg of said one or more compounds of Formula (V) or pharmaceutically acceptable salts, solvates, or hydrates thereof.

In some embodiments, the compound of Formula (IV) is selected from:

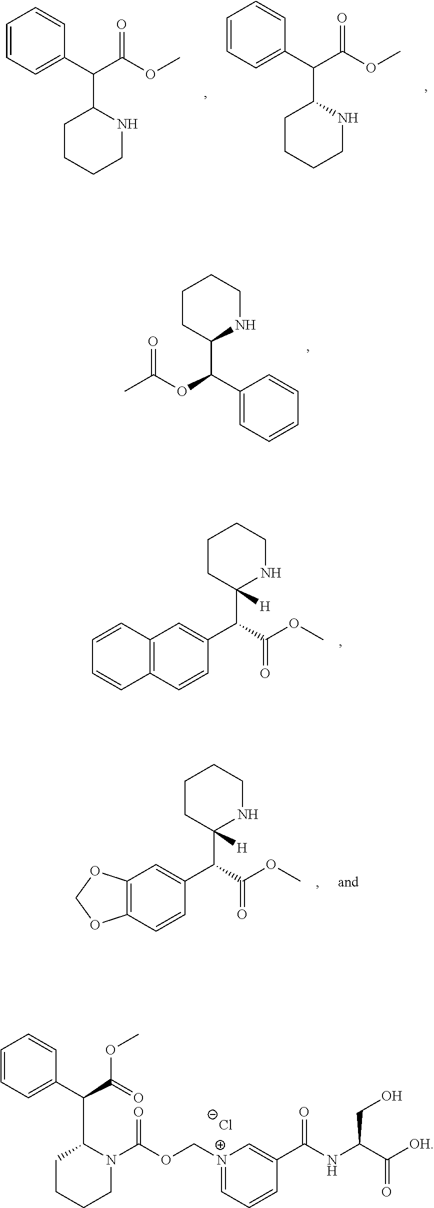

In some embodiments, the compound of Formula (V) is selected from:

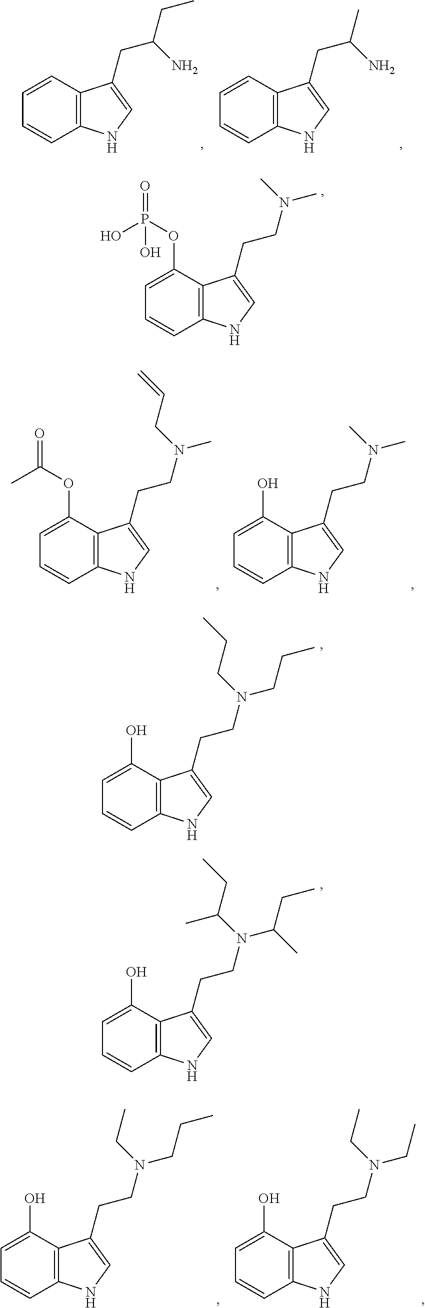

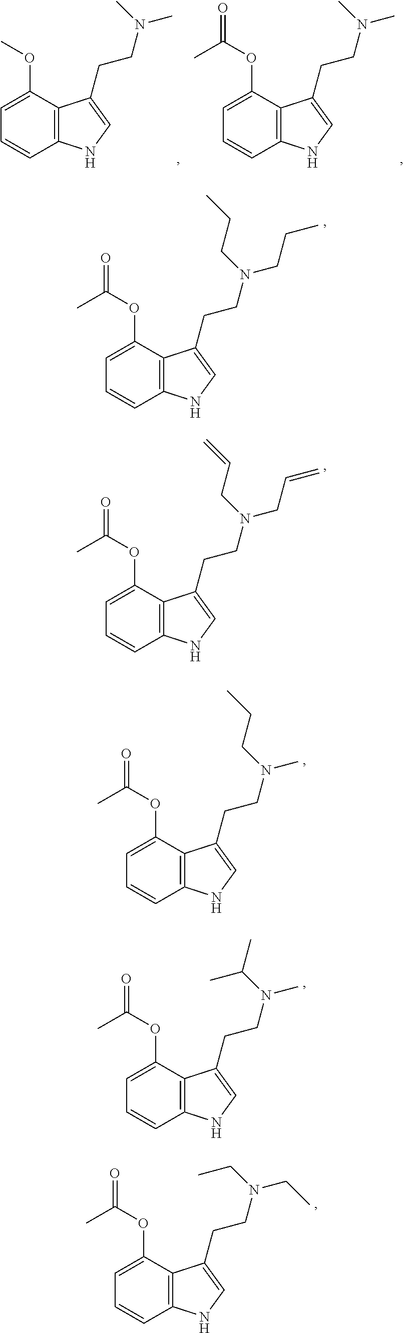

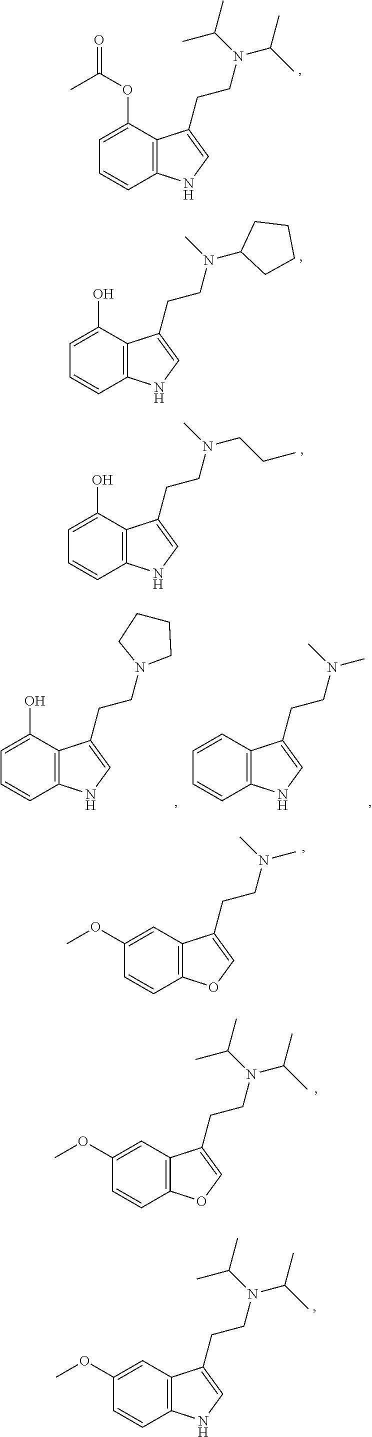

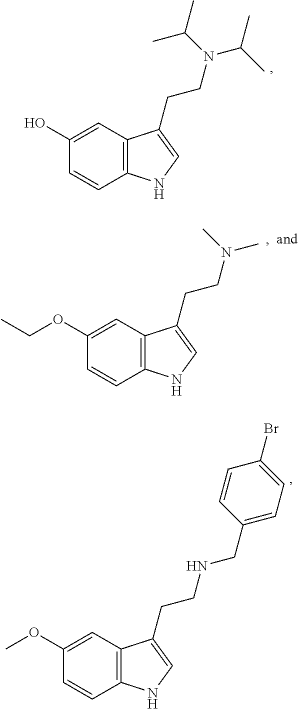

or pharmaceutically acceptable salts, solvates, or hydrates thereof.

In some embodiments, the combination further comprises one or more compounds of Formula (III)

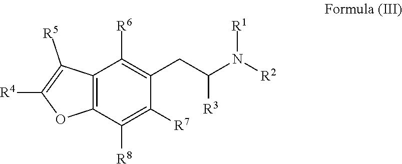

or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂;
R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

In some embodiments, for a compound of Formula (III), R¹ and R² are independently selected from C₁₋₆alkyl and C₂₋₆alkenyl. In some embodiments, for a compound of Formula (III), R¹ is C₁₋₆alkyl. In some embodiments, for a compound of Formula (III), R¹ is C₂₋₆alkenyl. In some embodiments, for a compound of Formula (III), R² is C₁₋₆alkyl. In some embodiments, for a compound of Formula (III), R² is C₂₋₆alkenyl. In some embodiments, for a compound of Formula (III), R¹ is C₁₋₆alkyl and R² is C₁₋₆alkyl. In some embodiments, for a compound of Formula (III), R¹ is C₂₋₆alkenyl and R² is C₁₋₆alkyl. In some embodiments, for a compound of Formula (III), R¹ is C₁₋₆alkyl and R² is C₂₋₆alkenyl. In some embodiments, for a compound of Formula (III), R¹ is C₂₋₆alkenyl and R² is C₂₋₆alkenyl. In some embodiments, for a compound of Formula (III), R¹ is methyl and R² is allyl. In some embodiments, for a compound of Formula (III), R¹ is methyl and R² is allyl. In some embodiments, for a compound of Formula (III), R¹ is methyl and R² is ethyl. In some embodiments, for a compound of Formula (III), R¹ is ethyl and R² is methyl. In some embodiments, for a compound of Formula (III), R¹ is methyl and R² is isopropyl. In some embodiments, for a compound of Formula (III), R¹ is isopropyl and R² is methyl. In some embodiments, for a compound of Formula (III), R¹ is isopropyl and R² is isopropyl. In some embodiments, for a compound of Formula (III), R¹ is methyl and R² is n-propyl. In some embodiments, for a compound of Formula (III), R¹ is n-propyl and R² is methyl.

In some embodiments, for a compound of Formula (III), R³ is selected from hydrogen, halo, C₁₋₆alkyl, C₁₋₆haloalkyl, C₁₋₆alkoxy, C₁₋₆haloalkoxy, and —N(R⁹)₂. In some embodiments, for a compound of Formula (III), R³ is selected from hydrogen, halo, C₁₋₆alkyl, and C₁₋₆haloalkyl. In some embodiments, for a compound of Formula (III), R³ is selected from hydrogen and C₁₋₆alkyl. In some embodiments, for a compound of Formula (III), R³ is hydrogen. In some embodiments, for a compound of Formula (III), R³ is C₁₋₆alkyl. In some embodiments, for a compound of Formula (III), R³ is methyl.

In some embodiments, for a compound of Formula (III), R⁴, R⁵, R⁶, R⁷, and R⁸ are independently selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹. In some embodiments, for a compound of Formula (III), R⁴, R⁵, R⁶, R⁷, and R⁸ are independently selected from hydrogen, halo, C₁₋₆alkyl, C₁₋₆haloalkyl, C₁₋₆alkoxy, —OR⁹, —OC(O)R⁹, —C(O)R⁹, and —C(O)OR⁹. In some embodiments, for a compound of Formula (III), R⁴, R⁵, R⁶, R⁷, and R⁸ are independently selected from hydrogen, halo, C₁₋₆alkoxy, —OR⁹, and —OC(O)R⁹. In some embodiments, for a compound of Formula (III), R⁴, R⁵, R⁶, R⁷, and R⁸ are independently selected from hydrogen, C₁₋₆alkoxy, —OR⁹, and —OC(O)R⁹. In some embodiments, for a compound of Formula (III), R⁴ and R⁸ are hydrogen, and R⁵ and R⁶ are independently selected from hydrogen, C₁₋₆alkoxy, —OR⁹, and —OC(O)R⁹. In some embodiments, for a compound of Formula (III), R⁴, R⁶, and R⁸ are hydrogen, and R⁵ is —OH. In some embodiments, for a compound of Formula (III), R⁴, R⁶, and R⁸ are hydrogen, and R⁵ is —OC(O)Me. In some embodiments, for a compound of Formula (III), R⁴, R⁵, and R⁸ are hydrogen, and R⁶ is —OH. In some embodiments, for a compound of Formula (III), R⁴, R⁵, and R⁸ are hydrogen, and R⁶ is —OMe.

In some embodiments, for a compound of Formula (III), R⁹ is selected from hydrogen, C₁₋₆alkyl, C₁₋₆haloalkyl C₃₋₆cycloalkyl, and C₆₋₁₀aryl. In some embodiments, for a compound of Formula (III), R⁹ is selected from hydrogen, C₁₋₆alkyl, and C₁₋₆haloalkyl. In some embodiments, for a compound of Formula (III), R⁹ is selected from hydrogen and C₁₋₆alkyl. In some embodiments, for a compound of Formula (III), R⁹ is hydrogen. In some embodiments, for a compound of Formula (III), R⁹ is C₁₋₆alkyl. In some embodiments, for a compound of Formula (III), R⁹ is methyl.

In some embodiments, the combination comprises about 1 mg to about 25 mg of said one or more compounds of Formula (III) or pharmaceutically acceptable salts, solvates, or hydrates thereof. In some embodiments, the combination comprises about 1 mg to about 10 mg of said one or more compounds of Formula (III) or pharmaceutically acceptable salts, solvates, or hydrates thereof. In some embodiments, the combination comprises about 10 mg to about 25 mg of said one or more compounds of Formula (III) or pharmaceutically acceptable salts, solvates, or hydrates thereof. In some embodiments, the combination comprises about 1 mg to about 5 mg of said one or more compounds of Formula (III) or pharmaceutically acceptable salts, solvates, or hydrates thereof. In some embodiments, the combination comprises about 5 mg to about 10 mg of said one or more compounds of Formula (III) or pharmaceutically acceptable salts, solvates, or hydrates thereof. In some embodiments, the combination comprises about 10 mg to about 15 mg of said one or more compounds of Formula (III) or pharmaceutically acceptable salts, solvates, or hydrates thereof. In some embodiments, the combination comprises about 15 mg to about 20 mg of said one or more compounds of Formula (III) or pharmaceutically acceptable salts, solvates, or hydrates thereof. In some embodiments, the combination comprises about 20 mg to about 25 mg of said one or more compounds of Formula (III) or pharmaceutically acceptable salts, solvates, or hydrates thereof. In some embodiments, the combination comprises about 25 mg to about 30 mg of said one or more compounds of Formula (III) or pharmaceutically acceptable salts, solvates, or hydrates thereof. In some embodiments, the combination comprises about 30 mg to about 35 mg of said one or more compounds of Formula (III) or pharmaceutically acceptable salts, solvates, or hydrates thereof. In some embodiments, the combination comprises about 35 mg to about 40 mg of said one or more compounds of Formula (III) or pharmaceutically acceptable salts, solvates, or hydrates thereof. In some embodiments, the combination comprises about 40 mg to about 45 mg of said one or more compounds of Formula (III) or pharmaceutically acceptable salts, solvates, or hydrates thereof. In some embodiments, the combination comprises about 45 mg to about 50 mg of said one or more compounds of Formula (III) or pharmaceutically acceptable salts, solvates, or hydrates thereof. In some embodiments, the combination comprises about 50 mg to about 55 mg of said one or more compounds of Formula (III) or pharmaceutically acceptable salts, solvates, or hydrates thereof. In some embodiments, the combination comprises about 55 mg to about 60 mg of said one or more compounds of Formula (III) or pharmaceutically acceptable salts, solvates, or hydrates thereof. In some embodiments, the combination comprises about 60 mg to about 65 mg of said one or more compounds of Formula (III) or pharmaceutically acceptable salts, solvates, or hydrates thereof. In some embodiments, the combination comprises about 65 mg to about 70 mg of said one or more compounds of Formula (III) or pharmaceutically acceptable salts, solvates, or hydrates thereof. In some embodiments, the combination comprises about 70 mg to about 75 mg of said one or more compounds of Formula (III) or pharmaceutically acceptable salts, solvates, or hydrates thereof. In some embodiments, the combination comprises about 1 mg of said one or more compounds of Formula (III) or pharmaceutically acceptable salts, solvates, or hydrates thereof. In some embodiments, the combination comprises about 2 mg of said one or more compounds of Formula (III) or pharmaceutically acceptable salts, solvates, or hydrates thereof. In some embodiments, the combination comprises about 3 mg of said one or more compounds of Formula (III) or pharmaceutically acceptable salts, solvates, or hydrates thereof. In some embodiments, the combination comprises about 4 mg of said one or more compounds of Formula (III) or pharmaceutically acceptable salts, solvates, or hydrates thereof. In some embodiments, the combination comprises about 5 mg of said one or more compounds of Formula (III) or pharmaceutically acceptable salts, solvates, or hydrates thereof. In some embodiments, the combination comprises about 6 mg of said one or more compounds of Formula (III) or pharmaceutically acceptable salts, solvates, or hydrates thereof. In some embodiments, the combination comprises about 7 mg of said one or more compounds of Formula (III) or pharmaceutically acceptable salts, solvates, or hydrates thereof. In some embodiments, the combination comprises about 8 mg of said one or more compounds of Formula (III) or pharmaceutically acceptable salts, solvates, or hydrates thereof. In some embodiments, the combination comprises about 9 mg of said one or more compounds of Formula (III) or pharmaceutically acceptable salts, solvates, or hydrates thereof. In some embodiments, the combination comprises about 10 mg of said one or more compounds of Formula (III) or pharmaceutically acceptable salts, solvates, or hydrates thereof. In some embodiments, the combination comprises about 11 mg of said one or more compounds of Formula (III) or pharmaceutically acceptable salts, solvates, or hydrates thereof. In some embodiments, the combination comprises about 12 mg of said one or more compounds of Formula (III) or pharmaceutically acceptable salts, solvates, or hydrates thereof. In some embodiments, the combination comprises about 13 mg of said one or more compounds of Formula (III) or pharmaceutically acceptable salts, solvates, or hydrates thereof. In some embodiments, the combination comprises about 14 mg of said one or more compounds of Formula (III) or pharmaceutically acceptable salts, solvates, or hydrates thereof. In some embodiments, the combination comprises about 15 mg of said one or more compounds of Formula (III) or pharmaceutically acceptable salts, solvates, or hydrates thereof. In some embodiments, the combination comprises about 16 mg of said one or more compounds of Formula (III) or pharmaceutically acceptable salts, solvates, or hydrates thereof. In some embodiments, the combination comprises about 17 mg of said one or more compounds of Formula (III) or pharmaceutically acceptable salts, solvates, or hydrates thereof. In some embodiments, the combination comprises about 18 mg of said one or more compounds of Formula (III) or pharmaceutically acceptable salts, solvates, or hydrates thereof. In some embodiments, the combination comprises about 19 mg of said one or more compounds of Formula (III) or pharmaceutically acceptable salts, solvates, or hydrates thereof. In some embodiments, the combination comprises about 20 mg of said one or more compounds of Formula (III) or pharmaceutically acceptable salts, solvates, or hydrates thereof. In some embodiments, the combination comprises about 21 mg of said one or more compounds of Formula (III) or pharmaceutically acceptable salts, solvates, or hydrates thereof. In some embodiments, the combination comprises about 22 mg of said one or more compounds of Formula (III) or pharmaceutically acceptable salts, solvates, or hydrates thereof. In some embodiments, the combination comprises about 23 mg of said one or more compounds of Formula (III) or pharmaceutically acceptable salts, solvates, or hydrates thereof. In some embodiments, the combination comprises about 24 mg of said one or more compounds of Formula (III) or pharmaceutically acceptable salts, solvates, or hydrates thereof. In some embodiments, the combination comprises about 25 mg of said one or more compounds of Formula (III) or pharmaceutically acceptable salts, solvates, or hydrates thereof. In some embodiments, the combination comprises about 30 mg of said one or more compounds of Formula (III) or pharmaceutically acceptable salts, solvates, or hydrates thereof. In some embodiments, the combination comprises about 35 mg of said one or more compounds of Formula (III) or pharmaceutically acceptable salts, solvates, or hydrates thereof. In some embodiments, the combination comprises about 40 mg of said one or more compounds of Formula (III) or pharmaceutically acceptable salts, solvates, or hydrates thereof. In some embodiments, the combination comprises about 45 mg of said one or more compounds of Formula (III) or pharmaceutically acceptable salts, solvates, or hydrates thereof. In some embodiments, the combination comprises about 50 mg of said one or more compounds of Formula (III) or pharmaceutically acceptable salts, solvates, or hydrates thereof. In some embodiments, the combination comprises about 55 mg of said one or more compounds of Formula (III) or pharmaceutically acceptable salts, solvates, or hydrates thereof. In some embodiments, the combination comprises about 60 mg of said one or more compounds of Formula (III) or pharmaceutically acceptable salts, solvates, or hydrates thereof. In some embodiments, the combination comprises about 65 mg of said one or more compounds of Formula (III) or pharmaceutically acceptable salts, solvates, or hydrates thereof. In some embodiments, the combination comprises about 70 mg of said one or more compounds of Formula (III) or pharmaceutically acceptable salts, solvates, or hydrates thereof. In some embodiments, the combination comprises about 75 mg of said one or more compounds of Formula (III) or pharmaceutically acceptable salts, solvates, or hydrates thereof.

In some embodiments, the compound of Formula (III) is selected from

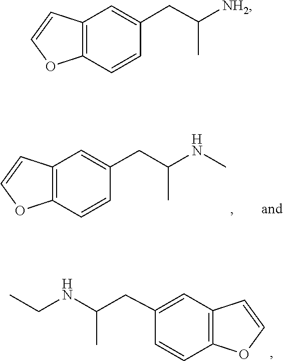

or pharmaceutically acceptable salts, solvates, or hydrates thereof.

In some embodiments, the combination further comprises one or more compounds of Formula (VI)

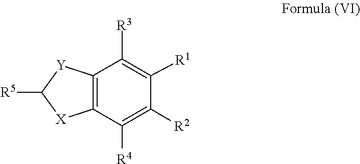

or pharmaceutically acceptable salts, solvates, or hydrates thereof, wherein
X and Y are independently selected from O and CH₂;
R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶;
R³ and R⁴ are independently selected from hydrogen, halogen, C₁₋₆alkyl, C₁₋₆haloalkyl, C₁₋₆alkoxy, C₁₋₆haloalkoxy, —N(R⁶)₂, —OR⁶, —OC(O)R⁶, —C(O)R⁶, and —C(O)OR⁶;
R⁵ is selected from hydrogen, halogen, —N(R⁶)₂, and —OR⁶; and
R⁶ is selected from hydrogen, C₁₋₆alkyl, C₁₋₆haloalkyl C₃₋₆cycloalkyl, and C₆₋₁₀aryl.

In some embodiments of the compound of Formula (VI), X is O. In some embodiments of the compound of Formula (VI), X is CH₂.

In some embodiments of the compound of Formula (VI), Y is O. In some embodiments of the compound of Formula (VI), Y is CH₂.

In some embodiments of the compound of Formula (VI), R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶. In some embodiments of the compound of Formula (VI), R¹ and R² are independently C₁₋₆alkyl, wherein the C₁₋₆alkyl is optionally substituted with —N(R⁶)₂ and —N(R⁶)OH. In some embodiments of the compound of Formula (VI), R¹ and R² are independently C₁₋₆alkyl, wherein the C₁₋₆alkyl is optionally substituted with —NH₂. In some embodiments of the compound of Formula (VI), R¹ and R² are independently C₁₋₆alkyl, wherein the C₁₋₆alkyl is optionally substituted with —NH(CH₃). In some embodiments of the compound of Formula (VI), R¹ and R² are independently hydrogen. In some embodiments of the compound of Formula (VI), R¹ and R² are independently halogen.

In some embodiments of the compound of Formula (VI), R³ and R⁴ are independently hydrogen.

In some embodiments of the compound of Formula (VI), R⁵ is selected from hydrogen, halogen, and —N(R⁶)₂. In some embodiments of the compound of Formula (VI), R⁵ is hydrogen. In some embodiments of the compound of Formula (VI), R⁵ is halogen. In some embodiments of the compound of Formula (VI), R⁵ is —N(R⁶)₂. In some embodiments of the compound of Formula (VI), R⁵ is —NH₂

In some embodiments of the compound of Formula (VI), R⁶ is hydrogen. In some embodiments of the compound of Formula (VI), R⁶ is C₁₋₆alkyl. In some embodiments of the compound of Formula (VI), R⁶ is methyl. In some embodiments of the compound of Formula (VI), R⁶ is ethyl.

In some embodiments, the combination comprises about 1 mg to about 25 mg of said one or more compounds of Formula (VI) or pharmaceutically acceptable salts, solvates, or hydrates thereof. In some embodiments, the combination comprises about 1 mg to about 10 mg of said one or more compounds of Formula (VI) or pharmaceutically acceptable salts, solvates, or hydrates thereof. In some embodiments, the combination comprises about 10 mg to about 25 mg of said one or more compounds of Formula (VI) or pharmaceutically acceptable salts, solvates, or hydrates thereof. In some embodiments, the combination comprises about 1 mg to about 5 mg of said one or more compounds of Formula (VI) or pharmaceutically acceptable salts, solvates, or hydrates thereof. In some embodiments, the combination comprises about 5 mg to about 10 mg of said one or more compounds of Formula (VI) or pharmaceutically acceptable salts, solvates, or hydrates thereof. In some embodiments, the combination comprises about 10 mg to about 15 mg of said one or more compounds of Formula (VI) or pharmaceutically acceptable salts, solvates, or hydrates thereof. In some embodiments, the combination comprises about 15 mg to about 20 mg of said one or more compounds of Formula (VI) or pharmaceutically acceptable salts, solvates, or hydrates thereof. In some embodiments, the combination comprises about 20 mg to about 25 mg of said one or more compounds of Formula (VI) or pharmaceutically acceptable salts, solvates, or hydrates thereof. In some embodiments, the combination comprises about 25 mg to about 30 mg of said one or more compounds of Formula (VI) or pharmaceutically acceptable salts, solvates, or hydrates thereof. In some embodiments, the combination comprises about 30 mg to about 35 mg of said one or more compounds of Formula (VI) or pharmaceutically acceptable salts, solvates, or hydrates thereof. In some embodiments, the combination comprises about 35 mg to about 40 mg of said one or more compounds of Formula (VI) or pharmaceutically acceptable salts, solvates, or hydrates thereof. In some embodiments, the combination comprises about 40 mg to about 45 mg of said one or more compounds of Formula (VI) or pharmaceutically acceptable salts, solvates, or hydrates thereof. In some embodiments, the combination comprises about 45 mg to about 50 mg of said one or more compounds of Formula (VI) or pharmaceutically acceptable salts, solvates, or hydrates thereof. In some embodiments, the combination comprises about 50 mg to about 55 mg of said one or more compounds of Formula (VI) or pharmaceutically acceptable salts, solvates, or hydrates thereof. In some embodiments, the combination comprises about 55 mg to about 60 mg of said one or more compounds of Formula (VI) or pharmaceutically acceptable salts, solvates, or hydrates thereof. In some embodiments, the combination comprises about 60 mg to about 65 mg of said one or more compounds of Formula (VI) or pharmaceutically acceptable salts, solvates, or hydrates thereof. In some embodiments, the combination comprises about 65 mg to about 70 mg of said one or more compounds of Formula (VI) or pharmaceutically acceptable salts, solvates, or hydrates thereof. In some embodiments, the combination comprises about 70 mg to about 75 mg of said one or more compounds of Formula (VI) or pharmaceutically acceptable salts, solvates, or hydrates thereof. In some embodiments, the combination comprises about 1 mg of said one or more compounds of Formula (VI) or pharmaceutically acceptable salts, solvates, or hydrates thereof. In some embodiments, the combination comprises about 2 mg of said one or more compounds of Formula (VI) or pharmaceutically acceptable salts, solvates, or hydrates thereof. In some embodiments, the combination comprises about 3 mg of said one or more compounds of Formula (VI) or pharmaceutically acceptable salts, solvates, or hydrates thereof. In some embodiments, the combination comprises about 4 mg of said one or more compounds of Formula (VI) or pharmaceutically acceptable salts, solvates, or hydrates thereof. In some embodiments, the combination comprises about 5 mg of said one or more compounds of Formula (VI) or pharmaceutically acceptable salts, solvates, or hydrates thereof. In some embodiments, the combination comprises about 6 mg of said one or more compounds of Formula (VI) or pharmaceutically acceptable salts, solvates, or hydrates thereof. In some embodiments, the combination comprises about 7 mg of said one or more compounds of Formula (VI) or pharmaceutically acceptable salts, solvates, or hydrates thereof. In some embodiments, the combination comprises about 8 mg of said one or more compounds of Formula (VI) or pharmaceutically acceptable salts, solvates, or hydrates thereof. In some embodiments, the combination comprises about 9 mg of said one or more compounds of Formula (VI) or pharmaceutically acceptable salts, solvates, or hydrates thereof. In some embodiments, the combination comprises about 10 mg of said one or more compounds of Formula (VI) or pharmaceutically acceptable salts, solvates, or hydrates thereof. In some embodiments, the combination comprises about 11 mg of said one or more compounds of Formula (VI) or pharmaceutically acceptable salts, solvates, or hydrates thereof. In some embodiments, the combination comprises about 12 mg of said one or more compounds of Formula (VI) or pharmaceutically acceptable salts, solvates, or hydrates thereof. In some embodiments, the combination comprises about 13 mg of said one or more compounds of Formula (VI) or pharmaceutically acceptable salts, solvates, or hydrates thereof. In some embodiments, the combination comprises about 14 mg of said one or more compounds of Formula (VI) or pharmaceutically acceptable salts, solvates, or hydrates thereof. In some embodiments, the combination comprises about 15 mg of said one or more compounds of Formula (VI) or pharmaceutically acceptable salts, solvates, or hydrates thereof. In some embodiments, the combination comprises about 16 mg of said one or more compounds of Formula (VI) or pharmaceutically acceptable salts, solvates, or hydrates thereof. In some embodiments, the combination comprises about 17 mg of said one or more compounds of Formula (VI) or pharmaceutically acceptable salts, solvates, or hydrates thereof. In some embodiments, the combination comprises about 18 mg of said one or more compounds of Formula (VI) or pharmaceutically acceptable salts, solvates, or hydrates thereof. In some embodiments, the combination comprises about 19 mg of said one or more compounds of Formula (VI) or pharmaceutically acceptable salts, solvates, or hydrates thereof. In some embodiments, the combination comprises about 20 mg of said one or more compounds of Formula (VI) or pharmaceutically acceptable salts, solvates, or hydrates thereof. In some embodiments, the combination comprises about 21 mg of said one or more compounds of Formula (VI) or pharmaceutically acceptable salts, solvates, or hydrates thereof. In some embodiments, the combination comprises about 22 mg of said one or more compounds of Formula (VI) or pharmaceutically acceptable salts, solvates, or hydrates thereof. In some embodiments, the combination comprises about 23 mg of said one or more compounds of Formula (VI) or pharmaceutically acceptable salts, solvates, or hydrates thereof. In some embodiments, the combination comprises about 24 mg of said one or more compounds of Formula (VI) or pharmaceutically acceptable salts, solvates, or hydrates thereof. In some embodiments, the combination comprises about 25 mg of said one or more compounds of Formula (VI) or pharmaceutically acceptable salts, solvates, or hydrates thereof. In some embodiments, the combination comprises about 30 mg of said one or more compounds of Formula (VI) or pharmaceutically acceptable salts, solvates, or hydrates thereof. In some embodiments, the combination comprises about 35 mg of said one or more compounds of Formula (VI) or pharmaceutically acceptable salts, solvates, or hydrates thereof. In some embodiments, the combination comprises about 40 mg of said one or more compounds of Formula (VI) or pharmaceutically acceptable salts, solvates, or hydrates thereof. In some embodiments, the combination comprises about 45 mg of said one or more compounds of Formula (VI) or pharmaceutically acceptable salts, solvates, or hydrates thereof. In some embodiments, the combination comprises about 50 mg of said one or more compounds of Formula (VI) or pharmaceutically acceptable salts, solvates, or hydrates thereof. In some embodiments, the combination comprises about 55 mg of said one or more compounds of Formula (VI) or pharmaceutically acceptable salts, solvates, or hydrates thereof. In some embodiments, the combination comprises about 60 mg of said one or more compounds of Formula (VI) or pharmaceutically acceptable salts, solvates, or hydrates thereof. In some embodiments, the combination comprises about 65 mg of said one or more compounds of Formula (VI) or pharmaceutically acceptable salts, solvates, or hydrates thereof. In some embodiments, the combination comprises about 70 mg of said one or more compounds of Formula (VI) or pharmaceutically acceptable salts, solvates, or hydrates thereof. In some embodiments, the combination comprises about 75 mg of said one or more compounds of Formula (VI) or pharmaceutically acceptable salts, solvates, or hydrates thereof.

In some embodiments, the combination further comprises one or more compounds of Formula (VII)

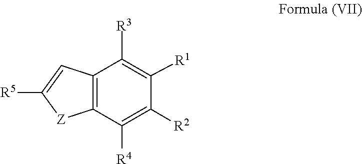

or pharmaceutically acceptable salts, solvates, or hydrates thereof, wherein
Z is selected from NH or O;
R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶;
R³ and R⁴ are independently selected from hydrogen, halogen, C₁₋₆alkyl, C₁₋₆haloalkyl, C₁₋₆ alkoxy, C₁₋₆haloalkoxy, —N(R⁶)₂, —OR⁶, —OC(O)R⁶, —C(O)R⁶, and —C(O)OR⁶;
R⁵ is selected from hydrogen, halogen, —N(R⁶)₂, and —OR⁶; and
R⁶ is selected from hydrogen, C₁₋₆alkyl, C₁₋₆haloalkyl C₃₋₆cycloalkyl, and C₆₋₁₀aryl.

In some embodiments of the compound of Formula (VII), Z is O. In some embodiments of the compound of Formula (VI), Z is NH.

In some embodiments of the compound of Formula (VII), R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶. In some embodiments of the compound of Formula (VII), R¹ and R² are independently C₁₋₆alkyl, wherein the C₁₋₆alkyl is optionally substituted with —N(R⁶)₂ and —N(R⁶)OH. In some embodiments of the compound of Formula (VII), R¹ and R² are independently C₁₋₆alkyl, wherein the C₁₋₆alkyl is optionally substituted with —NH₂. In some embodiments of the compound of Formula (VII), R¹ and R² are independently C₁₋₆alkyl, wherein the C₁₋₆alkyl is optionally substituted with —NH(CH₃).

In some embodiments of the compound of Formula (VII), R³ and R⁴ are independently hydrogen.

In some embodiments of the compound of Formula (VII), R⁵ is selected from hydrogen, halogen, and —N(R⁶)₂. In some embodiments of the compound of Formula (VII), R⁵ is hydrogen. In some embodiments of the compound of Formula (VII), R⁵ is halogen. In some embodiments of the compound of Formula (VII), R⁵ is —N(R⁶)₂.

In some embodiments of the compound of Formula (VII), R⁶ is hydrogen. In some embodiments of the compound of Formula (VII), R⁶ is C₁₋₆alkyl. In some embodiments of the compound of Formula (VII), R⁶ is methyl. In some embodiments of the compound of Formula (VII), R⁶ is ethyl.

In some embodiments, the combination comprises about 1 mg to about 25 mg of said one or more compounds of Formula (VII) or pharmaceutically acceptable salts, solvates, or hydrates thereof. In some embodiments, the combination comprises about 1 mg to about 10 mg of said one or more compounds of Formula (VII) or pharmaceutically acceptable salts, solvates, or hydrates thereof. In some embodiments, the combination comprises about 10 mg to about 25 mg of said one or more compounds of Formula (VII) or pharmaceutically acceptable salts, solvates, or hydrates thereof. In some embodiments, the combination comprises about 1 mg to about 5 mg of said one or more compounds of Formula (VII) or pharmaceutically acceptable salts, solvates, or hydrates thereof. In some embodiments, the combination comprises about 5 mg to about 10 mg of said one or more compounds of Formula (VII) or pharmaceutically acceptable salts, solvates, or hydrates thereof. In some embodiments, the combination comprises about 10 mg to about 15 mg of said one or more compounds of Formula (VII) or pharmaceutically acceptable salts, solvates, or hydrates thereof. In some embodiments, the combination comprises about 15 mg to about 20 mg of said one or more compounds of Formula (VII) or pharmaceutically acceptable salts, solvates, or hydrates thereof. In some embodiments, the combination comprises about 20 mg to about 25 mg of said one or more compounds of Formula (VII) or pharmaceutically acceptable salts, solvates, or hydrates thereof. In some embodiments, the combination comprises about 25 mg to about 30 mg of said one or more compounds of Formula (VII) or pharmaceutically acceptable salts, solvates, or hydrates thereof. In some embodiments, the combination comprises about 30 mg to about 35 mg of said one or more compounds of Formula (VII) or pharmaceutically acceptable salts, solvates, or hydrates thereof. In some embodiments, the combination comprises about 35 mg to about 40 mg of said one or more compounds of Formula (VII) or pharmaceutically acceptable salts, solvates, or hydrates thereof. In some embodiments, the combination comprises about 40 mg to about 45 mg of said one or more compounds of Formula (VII) or pharmaceutically acceptable salts, solvates, or hydrates thereof. In some embodiments, the combination comprises about 45 mg to about 50 mg of said one or more compounds of Formula (VII) or pharmaceutically acceptable salts, solvates, or hydrates thereof. In some embodiments, the combination comprises about 50 mg to about 55 mg of said one or more compounds of Formula (VII) or pharmaceutically acceptable salts, solvates, or hydrates thereof. In some embodiments, the combination comprises about 55 mg to about 60 mg of said one or more compounds of Formula (VII) or pharmaceutically acceptable salts, solvates, or hydrates thereof. In some embodiments, the combination comprises about 60 mg to about 65 mg of said one or more compounds of Formula (VII) or pharmaceutically acceptable salts, solvates, or hydrates thereof. In some embodiments, the combination comprises about 65 mg to about 70 mg of said one or more compounds of Formula (VII) or pharmaceutically acceptable salts, solvates, or hydrates thereof. In some embodiments, the combination comprises about 70 mg to about 75 mg of said one or more compounds of Formula (VII) or pharmaceutically acceptable salts, solvates, or hydrates thereof. In some embodiments, the combination comprises about 1 mg of said one or more compounds of Formula (VII) or pharmaceutically acceptable salts, solvates, or hydrates thereof. In some embodiments, the combination comprises about 2 mg of said one or more compounds of Formula (VII) or pharmaceutically acceptable salts, solvates, or hydrates thereof. In some embodiments, the combination comprises about 3 mg of said one or more compounds of Formula (VII) or pharmaceutically acceptable salts, solvates, or hydrates thereof. In some embodiments, the combination comprises about 4 mg of said one or more compounds of Formula (VII) or pharmaceutically acceptable salts, solvates, or hydrates thereof. In some embodiments, the combination comprises about 5 mg of said one or more compounds of Formula (VII) or pharmaceutically acceptable salts, solvates, or hydrates thereof. In some embodiments, the combination comprises about 6 mg of said one or more compounds of Formula (VII) or pharmaceutically acceptable salts, solvates, or hydrates thereof. In some embodiments, the combination comprises about 7 mg of said one or more compounds of Formula (VII) or pharmaceutically acceptable salts, solvates, or hydrates thereof. In some embodiments, the combination comprises about 8 mg of said one or more compounds of Formula (VII) or pharmaceutically acceptable salts, solvates, or hydrates thereof. In some embodiments, the combination comprises about 9 mg of said one or more compounds of Formula (VII) or pharmaceutically acceptable salts, solvates, or hydrates thereof. In some embodiments, the combination comprises about 10 mg of said one or more compounds of Formula (VII) or pharmaceutically acceptable salts, solvates, or hydrates thereof. In some embodiments, the combination comprises about 11 mg of said one or more compounds of Formula (VII) or pharmaceutically acceptable salts, solvates, or hydrates thereof. In some embodiments, the combination comprises about 12 mg of said one or more compounds of Formula (VII) or pharmaceutically acceptable salts, solvates, or hydrates thereof. In some embodiments, the combination comprises about 13 mg of said one or more compounds of Formula (VII) or pharmaceutically acceptable salts, solvates, or hydrates thereof. In some embodiments, the combination comprises about 14 mg of said one or more compounds of Formula (VII) or pharmaceutically acceptable salts, solvates, or hydrates thereof. In some embodiments, the combination comprises about 15 mg of said one or more compounds of Formula (VII) or pharmaceutically acceptable salts, solvates, or hydrates thereof. In some embodiments, the combination comprises about 16 mg of said one or more compounds of Formula (VII) or pharmaceutically acceptable salts, solvates, or hydrates thereof. In some embodiments, the combination comprises about 17 mg of said one or more compounds of Formula (VII) or pharmaceutically acceptable salts, solvates, or hydrates thereof. In some embodiments, the combination comprises about 18 mg of said one or more compounds of Formula (VII) or pharmaceutically acceptable salts, solvates, or hydrates thereof. In some embodiments, the combination comprises about 19 mg of said one or more compounds of Formula (VII) or pharmaceutically acceptable salts, solvates, or hydrates thereof. In some embodiments, the combination comprises about 20 mg of said one or more compounds of Formula (VII) or pharmaceutically acceptable salts, solvates, or hydrates thereof. In some embodiments, the combination comprises about 21 mg of said one or more compounds of Formula (VII) or pharmaceutically acceptable salts, solvates, or hydrates thereof. In some embodiments, the combination comprises about 22 mg of said one or more compounds of Formula (VII) or pharmaceutically acceptable salts, solvates, or hydrates thereof. In some embodiments, the combination comprises about 23 mg of said one or more compounds of Formula (VII) or pharmaceutically acceptable salts, solvates, or hydrates thereof. In some embodiments, the combination comprises about 24 mg of said one or more compounds of Formula (VII) or pharmaceutically acceptable salts, solvates, or hydrates thereof. In some embodiments, the combination comprises about 25 mg of said one or more compounds of Formula (VI) or pharmaceutically acceptable salts, solvates, or hydrates thereof. In some embodiments, the combination comprises about 30 mg of said one or more compounds of Formula (VII) or pharmaceutically acceptable salts, solvates, or hydrates thereof. In some embodiments, the combination comprises about 35 mg of said one or more compounds of Formula (VII) or pharmaceutically acceptable salts, solvates, or hydrates thereof. In some embodiments, the combination comprises about 40 mg of said one or more compounds of Formula (VII) or pharmaceutically acceptable salts, solvates, or hydrates thereof. In some embodiments, the combination comprises about 45 mg of said one or more compounds of Formula (VII) or pharmaceutically acceptable salts, solvates, or hydrates thereof. In some embodiments, the combination comprises about 50 mg of said one or more compounds of Formula (VII) or pharmaceutically acceptable salts, solvates, or hydrates thereof. In some embodiments, the combination comprises about 55 mg of said one or more compounds of Formula (VII) or pharmaceutically acceptable salts, solvates, or hydrates thereof. In some embodiments, the combination comprises about 60 mg of said one or more compounds of Formula (VII) or pharmaceutically acceptable salts, solvates, or hydrates thereof. In some embodiments, the combination comprises about 65 mg of said one or more compounds of Formula (VII) or pharmaceutically acceptable salts, solvates, or hydrates thereof. In some embodiments, the combination comprises about 70 mg of said one or more compounds of Formula (VII) or pharmaceutically acceptable salts, solvates, or hydrates thereof. In some embodiments, the combination comprises about 75 mg of said one or more compounds of Formula (VII) or pharmaceutically acceptable salts, solvates, or hydrates thereof.

In some embodiments, the compound of Formula (VI) is selected from

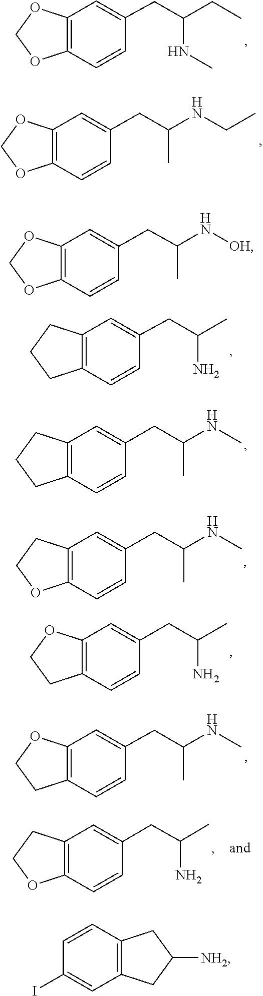

or pharmaceutically acceptable salts, solvates, or hydrates thereof.

In some embodiments, the compound of Formula VII is selected from:

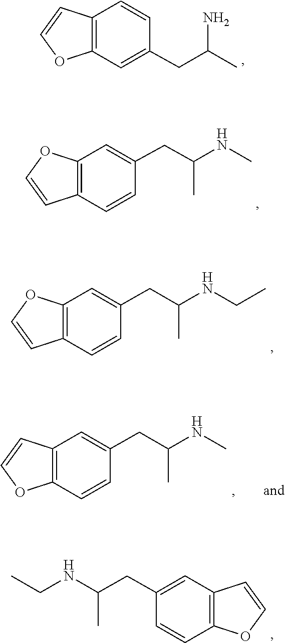

or pharmaceutically acceptable salts, solvates, or hydrates thereof.

In some embodiments, said one or more compounds of Formula (I) comprises one or more compounds of Formula (I-A)

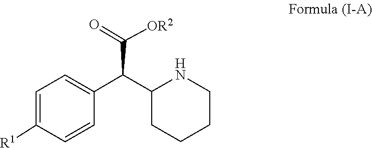

or pharmaceutically acceptable salts, solvates, or hydrates thereof, wherein
each R¹ is independently selected from hydrogen, halo, C₁₋₆alkyl, C₁₋₆haloalkyl, C₁₋₆alkoxy, C₁₋₆ haloalkoxy, —OR³, —C(O)R³, and —C(O)OR³;
R² is selected from hydrogen and C₁₋₆alkyl;
R³ is selected from hydrogen, C₁₋₆alkyl, C₃₋₆cycloalkyl, and C₆₋₁₀aryl; and
n is selected from 1, 2, 3, 4, and 5.

In some embodiments, said one or more compounds of Formula (II) comprises one or more compounds of Formula (II-A)

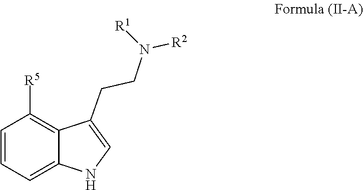

or pharmaceutically acceptable salts, solvates, or hydrates thereof, wherein
R¹ and R² are independently selected from C₁₋₆alkyl and C₂₋₆alkenyl;
R⁵ is selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

In some embodiments, said one or more compounds of Formula (III) comprises one or more compounds of Formula (III-A)

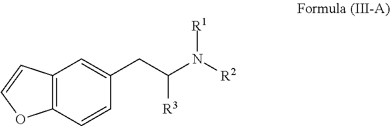

or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂; and
R⁹ is selected from hydrogen, C₁₋₆alkyl, C₁₋₆haloalkyl C₃₋₆cycloalkyl, and C₆₋₁₀aryl.

In some embodiments, said one or more compounds of Formula (I) comprise Compound 1

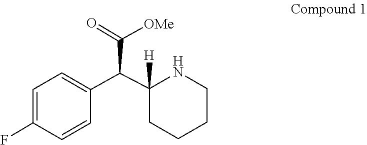 [SMILES: COC(=O)[C@H](c1ccc(F)cc1)[C@H]1CCCCN1]

or a pharmaceutically acceptable salt, solvate, or hydrate thereof.

In some embodiments, said one or more compounds of Formula (II) comprise Compound 2

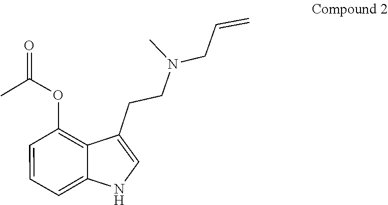 [SMILES: C=CCN(C)CCc1c[nH]c2cccc(OC(C)=O)c12]

or a pharmaceutically acceptable salt, solvate, or hydrate thereof.

In some embodiments, said one or more compounds of Formula (II) comprise Compound 2-A

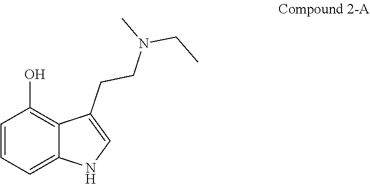 [SMILES: CCN(C)CCc1c[nH]c2cccc(O)c12]

or a pharmaceutically acceptable salt, solvate, or hydrate thereof.

In some embodiments, said one or more compounds of Formula (II) comprise Compound 2-B

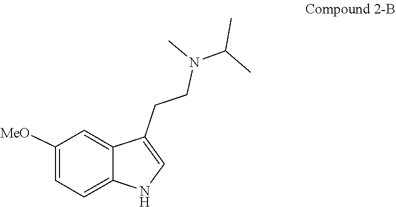 [SMILES: COc1ccc2[nH]cc(CCN(C)C(C)C)c2c1]

or a pharmaceutically acceptable salt, solvate, or hydrate thereof.

In some embodiments, said one or more compounds of Formula (II) comprise Compound 2-C

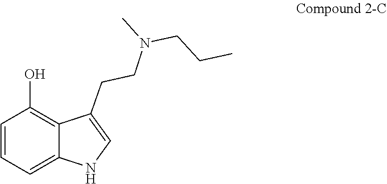 [SMILES: CCCN(C)CCc1c[nH]c2cccc(O)c12]

or a pharmaceutically acceptable salt, solvate, or hydrate thereof.

In some embodiments, said one or more compounds of Formula (II) comprise Compound 2-D

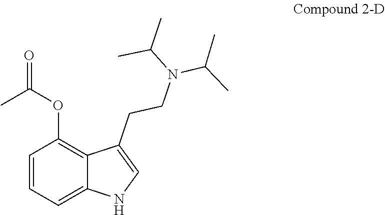 [SMILES: CC(=O)Oc1cccc2[nH]cc(CCN(C(C)C)C(C)C)c12]

or a pharmaceutically acceptable salt, solvate, or hydrate thereof.

In some embodiments, said one or more compounds of Formula (II) comprise Compound 2-E

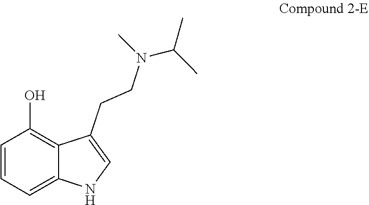 [SMILES: CC(C)N(C)CCc1c[nH]c2cccc(O)c12]

or a pharmaceutically acceptable salt, solvate, or hydrate thereof.

In some embodiments, said one or more compounds of Formula (II) comprise Compound 2-F

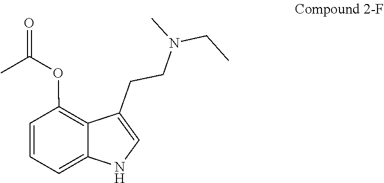 [SMILES: CCN(C)CCc1c[nH]c2cccc(OC(C)=O)c12]

or a pharmaceutically acceptable salt, solvate, or hydrate thereof.

In some embodiments, said one or more compounds of Formula (III) comprise Compound 3

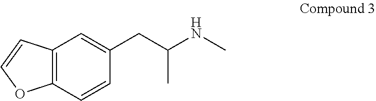

or a pharmaceutically acceptable salt, solvate, or hydrate thereof.

In some embodiments, said one or more compounds of Formula (III) comprise Compound 3-A

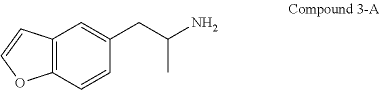

or a pharmaceutically acceptable salt, solvate, or hydrate thereof.

In another aspect, provided herein is a combination comprising two or more compounds useful for the treatment of one or more sexual disorders, or pharmaceutically acceptable salts, solvates, or hydrates thereof, wherein said two or more compounds comprise an optionally fused benzene ring.

In some embodiments, said combination comprises three or more compounds useful for the treatment of one or more sexual disorders, wherein said three or more compounds comprise an optionally fused benzene ring.

In some embodiments, said compounds are independently selected from phenethylamines, tryptamines, benzofurans, and amphetamines. In some embodiments, said combination comprises a phenethylamine, a tryptamine, and a benzofuran.

In some embodiments, the tryptamine is selected from

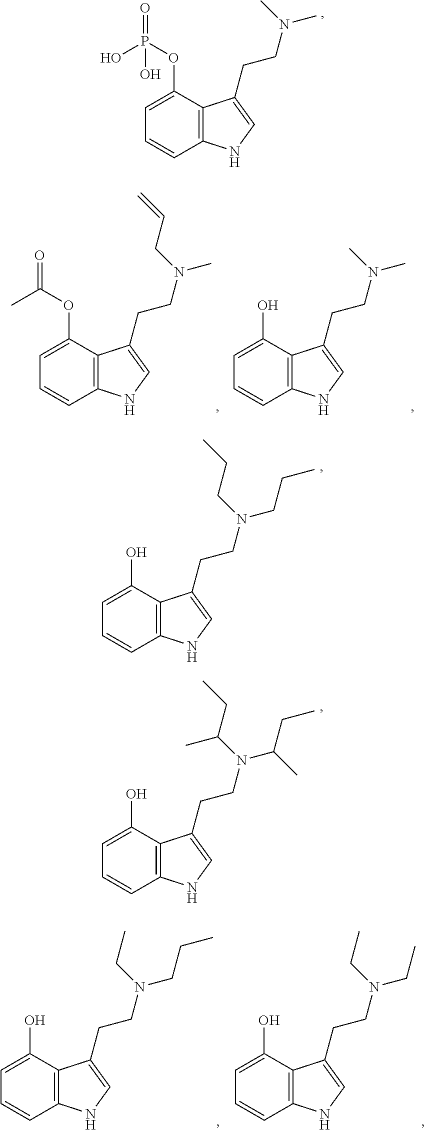

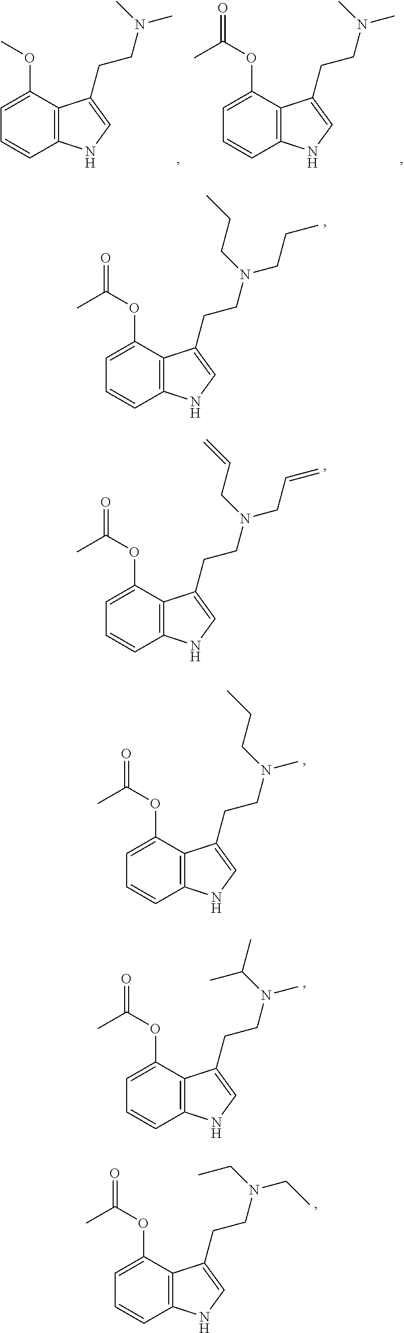

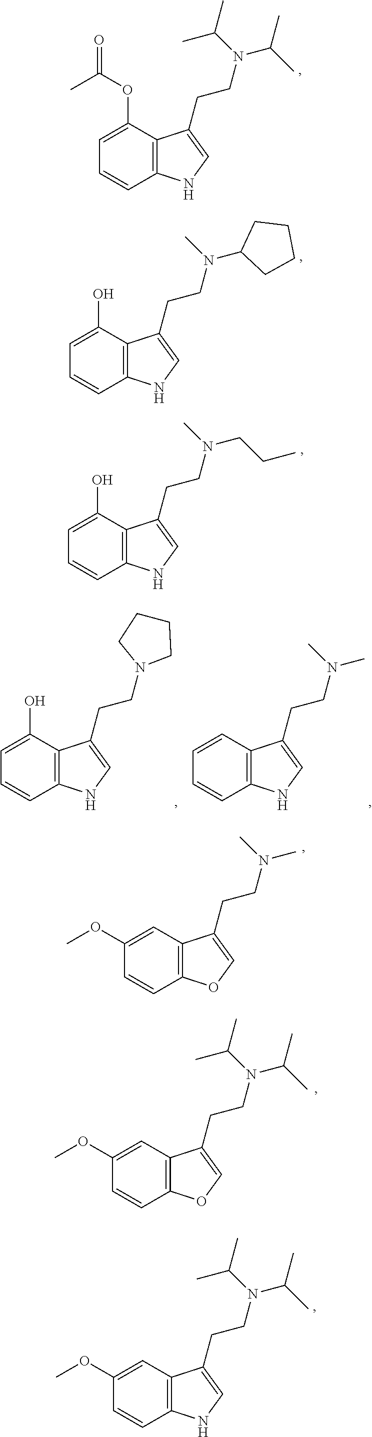

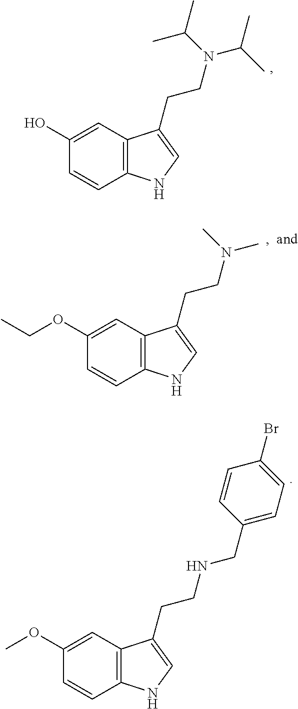

In some embodiments, the benzofuran is selected from

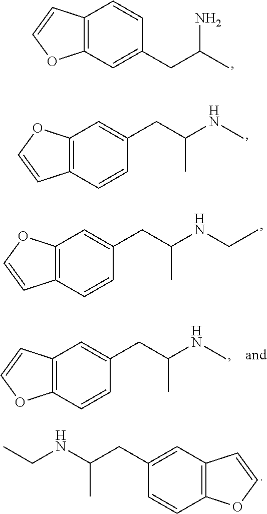

In some embodiments, the amphetamine is selected from

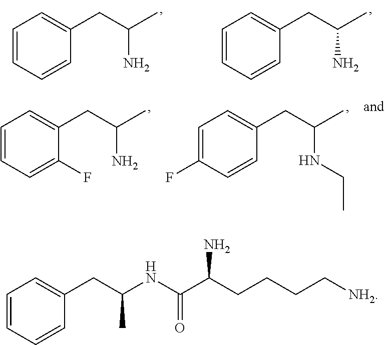

In some embodiments, at least one of said two or more compounds comprises Compound 1

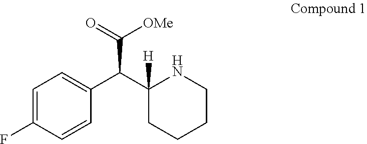 [SMILES: COC(=O)[C@H](c1ccc(F)cc1)[C@H]1CCCCN1]

or a pharmaceutically acceptable salt, solvate, or hydrate thereof.

In some embodiments, at least one of said two or more compounds comprises Compound 1-A

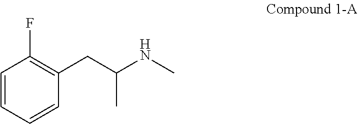

or a pharmaceutically acceptable salt, solvate, or hydrate thereof.

In some embodiments, at least one of said two or more compounds comprises Compound 2

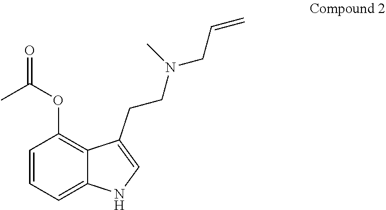 [SMILES: C=CCN(C)CCc1c[nH]c2cccc(OC(C)=O)c12]

or a pharmaceutically acceptable salt, solvate, or hydrate thereof.

In some embodiments, at least one of said two or more compounds comprises Compound 2-A

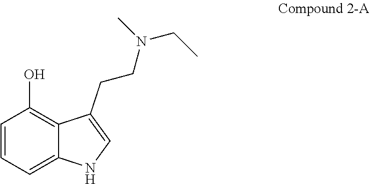 [SMILES: CCN(C)CCc1c[nH]c2cccc(O)c12]

or a pharmaceutically acceptable salt, solvate, or hydrate thereof.

In some embodiments, at least one of said two or more compounds comprises Compound 2-B

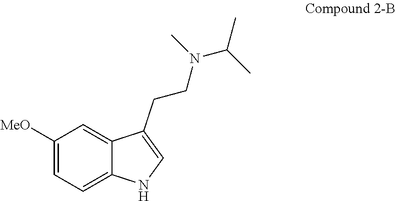 [SMILES: COc1ccc2[nH]cc(CCN(C)C(C)C)c2c1]

or a pharmaceutically acceptable salt, solvate, or hydrate thereof.

In some embodiments, at least one of said two or more compounds comprises Compound 2-C

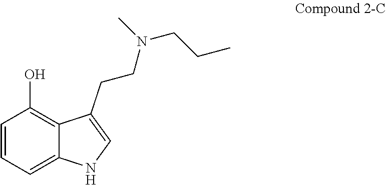 [SMILES: CCCN(C)CCc1c[nH]c2cccc(O)c12]

or a pharmaceutically acceptable salt, solvate, or hydrate thereof.

In some embodiments, at least one of said two or more compounds comprises Compound 2-D

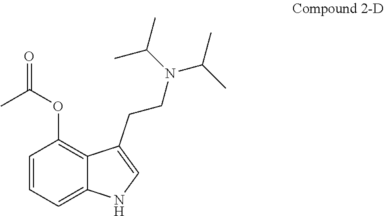 [SMILES: CC(=O)Oc1cccc2[nH]cc(CCN(C(C)C)C(C)C)c12]

or a pharmaceutically acceptable salt, solvate, or hydrate thereof.

In some embodiments, at least one of said two or more compounds comprises Compound 2-E

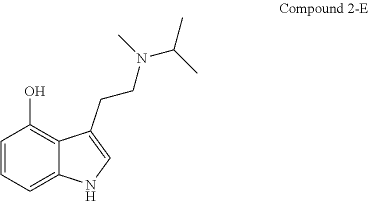 [SMILES: CC(C)N(C)CCc1c[nH]c2cccc(O)c12]

or a pharmaceutically acceptable salt, solvate, or hydrate thereof.

In some embodiments, at least one of said two or more compounds comprises Compound 2-F

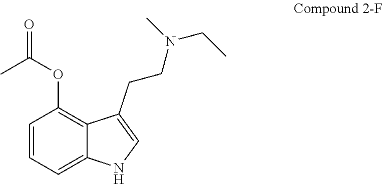 [SMILES: CCN(C)CCc1c[nH]c2cccc(OC(C)=O)c12]

or a pharmaceutically acceptable salt, solvate, or hydrate thereof.

In some embodiments, at least one of said two or more compounds comprises Compound

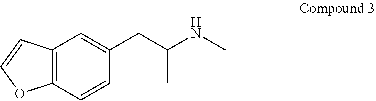

or a pharmaceutically acceptable salt, solvate, or hydrate thereof.

In some embodiments, at least one of said two or more compounds comprises Compound 3-A

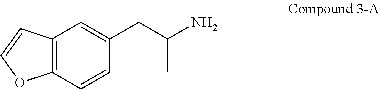

or a pharmaceutically acceptable salt, solvate, or hydrate thereof.

In some embodiments, at least one of said two or more compounds comprises Compound 3-B

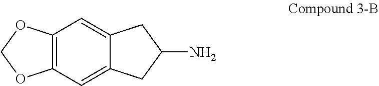 [SMILES: NC1Cc2cc3c(cc2C1)OCO3]

or a pharmaceutically acceptable salt, solvate, or hydrate thereof.

In some embodiments, Compound 1 or Compound 1-A, or a pharmaceutically acceptable salt, solvate, or hydrate thereof, is present in the combination at about 1 mg to about 20 mg. In some embodiments, Compound 1 or Compound 1-A, or a pharmaceutically acceptable salt, solvate, or hydrate thereof, is present in the combination at about 1 mg to about 10 mg. In some embodiments, Compound 1 or Compound 1-A, or a pharmaceutically acceptable salt, solvate, or hydrate thereof, is present in the combination at about 10 mg to about 20 mg. In some embodiments, Compound 1 or Compound 1-A, or a pharmaceutically acceptable salt, solvate, or hydrate thereof, is present in the combination at about 1 mg to about 5 mg. In some embodiments, Compound 1 or Compound 1-A, or a pharmaceutically acceptable salt, solvate, or hydrate thereof, is present in the combination at about 5 mg to about 10 mg. In some embodiments, Compound 1 or Compound 1-A, or a pharmaceutically acceptable salt, solvate, or hydrate thereof, is present in the combination at about 10 mg to about 15 mg. In some embodiments, Compound 1 or Compound 1-A, or a pharmaceutically acceptable salt, solvate, or hydrate thereof, is present in the combination at about 15 mg to about 20 mg. In some embodiments, Compound 1 or Compound 1-A, or a pharmaceutically acceptable salt, solvate, or hydrate thereof, is present in the combination at about 1 mg. In some embodiments, Compound 1 or Compound 1-A, or a pharmaceutically acceptable salt, solvate, or hydrate thereof, is present in the combination at about 2 mg. In some embodiments, Compound 1 or Compound 1-A, or a pharmaceutically acceptable salt, solvate, or hydrate thereof, is present in the combination at about 3 mg. In some embodiments, Compound 1 or Compound 1-A, or a pharmaceutically acceptable salt, solvate, or hydrate thereof, is present in the combination at about 4 mg. In some embodiments, Compound 1 or Compound 1-A, or a pharmaceutically acceptable salt, solvate, or hydrate thereof, is present in the combination at about 5 mg. In some embodiments, Compound 1 or Compound 1-A, or a pharmaceutically acceptable salt, solvate, or hydrate thereof, is present in the combination at about 6 mg. In some embodiments, Compound 1 or Compound 1-A, or a pharmaceutically acceptable salt, solvate, or hydrate thereof, is present in the combination at about 7 mg. In some embodiments, Compound 1 or Compound 1-A, or a pharmaceutically acceptable salt, solvate, or hydrate thereof, is present in the combination at about 8 mg. In some embodiments, Compound 1 or Compound 1-A, or a pharmaceutically acceptable salt, solvate, or hydrate thereof, is present in the combination at about 9 mg. In some embodiments, Compound 1 or Compound 1-A, or a pharmaceutically acceptable salt, solvate, or hydrate thereof, is present in the combination at about 10 mg. In some embodiments, Compound 1 or Compound 1-A, or a pharmaceutically acceptable salt, solvate, or hydrate thereof, is present in the combination at about 11 mg. In some embodiments, Compound 1 or Compound 1-A, or a pharmaceutically acceptable salt, solvate, or hydrate thereof, is present in the combination at about 12 mg. In some embodiments, Compound 1 or Compound 1-A, or a pharmaceutically acceptable salt, solvate, or hydrate thereof, is present in the combination at about 13 mg. In some embodiments, Compound 1 or Compound 1-A, or a pharmaceutically acceptable salt, solvate, or hydrate thereof, is present in the combination at about 14 mg. In some embodiments, Compound 1 or Compound 1-A, or a pharmaceutically acceptable salt, solvate, or hydrate thereof, is present in the combination at about 15 mg. In some embodiments, Compound 1 or Compound 1-A, or a pharmaceutically acceptable salt, solvate, or hydrate thereof, is present in the combination at about 16 mg. In some embodiments, Compound 1 or Compound 1-A, or a pharmaceutically acceptable salt, solvate, or hydrate thereof, is present in the combination at about 17 mg. In some embodiments, Compound 1 or Compound 1-A, or a pharmaceutically acceptable salt, solvate, or hydrate thereof, is present in the combination at about 18 mg. In some embodiments, Compound 1 or Compound 1-A, or a pharmaceutically acceptable salt, solvate, or hydrate thereof, is present in the combination at about 19 mg. In some embodiments, Compound 1 or Compound 1-A, or a pharmaceutically acceptable salt, solvate, or hydrate thereof, is present in the combination at about 20 mg.

In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 1 mg to about 25 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 1 mg to about 10 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 10 mg to about 25 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 1 mg to about 5 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 5 mg to about 10 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 10 mg to about 15 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 15 mg to about 20 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 20 mg to about 25 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 1 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 2 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 3 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 4 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 5 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 6 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 7 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 8 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 9 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 10 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 11 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 12 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 13 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 14 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 15 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 16 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 17 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 18 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 19 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 20 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 21 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 22 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 23 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 24 mg. In some embodiments, Compound 2, Compound 2-A, Compound 2-B, Compound 2-C, Compound 2-D, Compound 2-E, or Compound 2-F, or a pharmaceutically acceptable salt, solvate, or hydrate thereof, is present in the combination at about 25 mg.

In some embodiments, Compound 3, Compound 3-A, or Compound 3-B, or a pharmaceutically acceptable salt, solvate, or hydrate thereof, is present in the combination at about 1 mg to about 75 mg. In some embodiments, Compound 3, Compound 3-A, or Compound 3-B, or a pharmaceutically acceptable salt, solvate, or hydrate thereof, is present in the combination at about 1 mg to about 10 mg. In some embodiments, Compound 3, Compound 3-A, or Compound 3-B, or a pharmaceutically acceptable salt, solvate, or hydrate thereof, is present in the combination at about 10 mg to about 25 mg. In some embodiments, Compound 3, Compound 3-A, or Compound 3-B, or a pharmaceutically acceptable salt, solvate, or hydrate thereof, is present in the combination at about 1 mg to about 5 mg. In some embodiments, Compound 3, Compound 3-A, or Compound 3-B, or a pharmaceutically acceptable salt, solvate, or hydrate thereof, is present in the combination at about 5 mg to about 10 mg. In some embodiments, Compound 3, Compound 3-A, or Compound 3-B, or a pharmaceutically acceptable salt, solvate, or hydrate thereof, is present in the combination at about 10 mg to about 15 mg. In some embodiments, Compound 3, Compound 3-A, or Compound 3-B, or a pharmaceutically acceptable salt, solvate, or hydrate thereof, is present in the combination at about 15 mg to about 20 mg. In some embodiments, Compound 3, Compound 3-A, or Compound 3-B, or a pharmaceutically acceptable salt, solvate, or hydrate thereof, is present in the combination at about 20 mg to about 25 mg. In some embodiments, Compound 3, Compound 3-A, or Compound 3-B, or a pharmaceutically acceptable salt, solvate, or hydrate thereof, is present in the combination at about 1 mg. In some embodiments, Compound 3, Compound 3-A, or Compound 3-B, or a pharmaceutically acceptable salt, solvate, or hydrate thereof, is present in the combination at about 2 mg. In some embodiments, Compound 3, Compound 3-A, or Compound 3-B, or a pharmaceutically acceptable salt, solvate, or hydrate thereof, is present in the combination at about 3 mg. In some embodiments, Compound 3, Compound 3-A, or Compound 3-B, or a pharmaceutically acceptable salt, solvate, or hydrate thereof, is present in the combination at about 4 mg. In some embodiments, Compound 3, Compound 3-A, or Compound 3-B, or a pharmaceutically acceptable salt, solvate, or hydrate thereof, is present in the combination at about 5 mg. In some embodiments, Compound 3, Compound 3-A, or Compound 3-B, or a pharmaceutically acceptable salt, solvate, or hydrate thereof, is present in the combination at about 6 mg. In some embodiments, Compound 3, Compound 3-A, or Compound 3-B, or a pharmaceutically acceptable salt, solvate, or hydrate thereof, is present in the combination at about 7 mg. In some embodiments, Compound 3, Compound 3-A, or Compound 3-B, or a pharmaceutically acceptable salt, solvate, or hydrate thereof, is present in the combination at about 8 mg. In some embodiments, Compound 3, Compound 3-A, or Compound 3-B, or a pharmaceutically acceptable salt, solvate, or hydrate thereof, is present in the combination at about 9 mg. In some embodiments, Compound 3, Compound 3-A, or Compound 3-B, or a pharmaceutically acceptable salt, solvate, or hydrate thereof, is present in the combination at about 10 mg. In some embodiments, Compound 3, Compound 3-A, or Compound 3-B, or a pharmaceutically acceptable salt, solvate, or hydrate thereof, is present in the combination at about 11 mg. In some embodiments, Compound 3, Compound 3-A, or Compound 3-B, or a pharmaceutically acceptable salt, solvate, or hydrate thereof, is present in the combination at about 12 mg. In some embodiments, Compound 3, Compound 3-A, or Compound 3-B, or a pharmaceutically acceptable salt, solvate, or hydrate thereof, is present in the combination at about 13 mg. In some embodiments, Compound 3, Compound 3-A, or Compound 3-B, or a pharmaceutically acceptable salt, solvate, or hydrate thereof, is present in the combination at about 14 mg. In some embodiments, Compound 3, Compound 3-A, or Compound 3-B, or a pharmaceutically acceptable salt, solvate, or hydrate thereof, is present in the combination at about 15 mg. In some embodiments, Compound 3, Compound 3-A, or Compound 3-B, or a pharmaceutically acceptable salt, solvate, or hydrate thereof, is present in the combination at about 16 mg. In some embodiments, Compound 3, Compound 3-A, or Compound 3-B, or a pharmaceutically acceptable salt, solvate, or hydrate thereof, is present in the combination at about 17 mg. In some embodiments, Compound 3, Compound 3-A, or Compound 3-B, or a pharmaceutically acceptable salt, solvate, or hydrate thereof, is present in the combination at about 18 mg. In some embodiments, Compound 3, Compound 3-A, or Compound 3-B, or a pharmaceutically acceptable salt, solvate, or hydrate thereof, is present in the combination at about 19 mg. In some embodiments, Compound 3, Compound 3-A, or Compound 3-B, or a pharmaceutically acceptable salt, solvate, or hydrate thereof, is present in the combination at about 20 mg. In some embodiments, Compound 3, Compound 3-A, or Compound 3-B, or a pharmaceutically acceptable salt, solvate, or hydrate thereof, is present in the combination at about 21 mg. In some embodiments, Compound 3, Compound 3-A, or Compound 3-B, or a pharmaceutically acceptable salt, solvate, or hydrate thereof, is present in the combination at about 22 mg. In some embodiments, Compound 3, Compound 3-A, or Compound 3-B, or a pharmaceutically acceptable salt, solvate, or hydrate thereof, is present in the combination at about 23 mg. In some embodiments, Compound 3, Compound 3-A, or Compound 3-B, or a pharmaceutically acceptable salt, solvate, or hydrate thereof, is present in the combination at about 24 mg. In some embodiments, Compound 3, Compound 3-A, or Compound 3-B, or a pharmaceutically acceptable salt, solvate, or hydrate thereof, is present in the combination at about 25 mg. In some embodiments, Compound 3, Compound 3-A, or Compound 3-B, or a pharmaceutically acceptable salt, solvate, or hydrate thereof, is present in the combination at about 25 mg to about 75 mg. In some embodiments, Compound 3, Compound 3-A, or Compound 3-B, or a pharmaceutically acceptable salt, solvate, or hydrate thereof, is present in the combination at about 25 mg to about 30 mg, about 25 mg to about 35 mg, about 25 mg to about 40 mg, about 25 mg to about 45 mg, about 25 mg to about 50 mg, about 25 mg to about 55 mg, about 25 mg to about 60 mg, about 25 mg to about 65 mg, about 25 mg to about 70 mg, about 25 mg to about 75 mg, about 30 mg to about 35 mg, about 30 mg to about 40 mg, about 30 mg to about 45 mg, about 30 mg to about 50 mg, about 30 mg to about 55 mg, about 30 mg to about 60 mg, about 30 mg to about 65 mg, about 30 mg to about 70 mg, about 30 mg to about 75 mg, about 35 mg to about 40 mg, about 35 mg to about 45 mg, about 35 mg to about 50 mg, about 35 mg to about 55 mg, about 35 mg to about 60 mg, about 35 mg to about 65 mg, about 35 mg to about 70 mg, about 35 mg to about 75 mg, about 40 mg to about 45 mg, about 40 mg to about 50 mg, about 40 mg to about 55 mg, about 40 mg to about 60 mg, about 40 mg to about 65 mg, about 40 mg to about 70 mg, about 40 mg to about 75 mg, about 45 mg to about 50 mg, about 45 mg to about 55 mg, about 45 mg to about 60 mg, about 45 mg to about 65 mg, about 45 mg to about 70 mg, about 45 mg to about 75 mg, about 50 mg to about 55 mg, about 50 mg to about 60 mg, about 50 mg to about 65 mg, about 50 mg to about 70 mg, about 50 mg to about 75 mg, about 55 mg to about 60 mg, about 55 mg to about 65 mg, about 55 mg to about 70 mg, about 55 mg to about 75 mg, about 60 mg to about 65 mg, about 60 mg to about 70 mg, about 60 mg to about 75 mg, about 65 mg to about 70 mg, about 65 mg to about 75 mg, or about 70 mg to about 75 mg. In some embodiments, Compound 3, Compound 3-A, or Compound 3-B, or a pharmaceutically acceptable salt, solvate, or hydrate thereof, is present in the combination at about 25 mg, about 30 mg, about 35 mg, about 40 mg, about 45 mg, about 50 mg, about 55 mg, about 60 mg, about 65 mg, about 70 mg, or about 75 mg. In some embodiments, Compound 3, Compound 3-A, or Compound 3-B, or a pharmaceutically acceptable salt, solvate, or hydrate thereof, is present in the combination at least about 25 mg, about 30 mg, about 35 mg, about 40 mg, about 45 mg, about 50 mg, about 55 mg, about 60 mg, about 65 mg, or about 70 mg. In some embodiments, Compound 3, Compound 3-A, or Compound 3-B, or a pharmaceutically acceptable salt, solvate, or hydrate thereof, is present in the combination at most about 30 mg, about 35 mg, about 40 mg, about 45 mg, about 50 mg, about 55 mg, about 60 mg, about 65 mg, about 70 mg, or about 75 mg.

In another aspect, provided herein is a combination comprising at least two compounds independently selected from empathogens, stimulants, and tryptamines. In some embodiments, the combination comprises an empathogen, a stimulant, and a tryptamine.

In some embodiments, the empathogen is selected from Compound 3

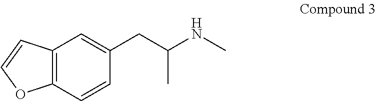

### and Compound 3-A

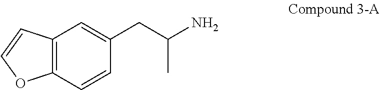

or a pharmaceutically acceptable salt, solvate, or hydrate thereof. In some embodiments, the empathogen is Compound 3 or a pharmaceutically acceptable salt, solvate, or hydrate thereof. In some embodiments, the empathogen is Compound 3-A or a pharmaceutically acceptable salt, solvate, or hydrate thereof.

In some embodiments, the stimulant is selected from Compound 1:

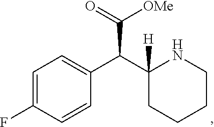 [SMILES: COC(=O)[C@H](c1ccc(F)cc1)[C@H]1CCCCN1]

### Compound 1-A:

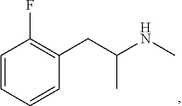

### and Compound 4:

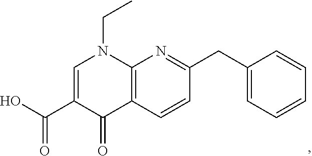 [SMILES: CCn1cc(C(=O)O)c(=O)c2ccc(Cc3ccccc3)nc21]

or a pharmaceutically acceptable salt, solvate, or hydrate thereof. In some embodiments, the stimulant is Compound 1 or a pharmaceutically acceptable salt, solvate, or hydrate thereof. In some embodiments, the stimulant is Compound 1-A or a pharmaceutically acceptable salt, solvate, or hydrate thereof. In some embodiments, the stimulant is Compound 4 (Amfonelic acid) or a pharmaceutically acceptable salt, solvate, or hydrate thereof.

In some embodiments, the stimulant is selected from Compound 1

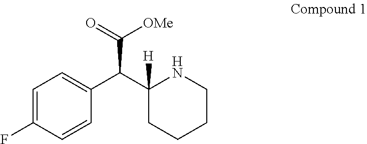 [SMILES: COC(=O)[C@H](c1ccc(F)cc1)[C@H]1CCCCN1]

### and Compound 1-A

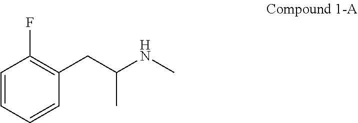

or a pharmaceutically acceptable salt, solvate, or hydrate thereof. In some embodiments, the stimulant is Compound 1 or a pharmaceutically acceptable salt, solvate, or hydrate thereof. In some embodiments, the stimulant is Compound 1-A or a pharmaceutically acceptable salt, solvate, or hydrate thereof.

In some embodiments, the tryptamine is selected from Compound 2,

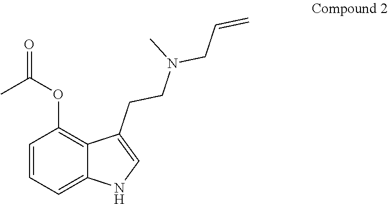 [SMILES: C=CCN(C)CCc1c[nH]c2cccc(OC(C)=O)c12]

### Compound 2-A,

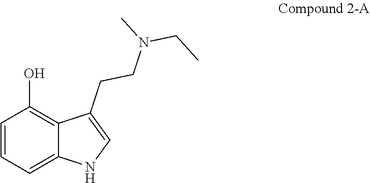 [SMILES: CCN(C)CCc1c[nH]c2cccc(O)c12]

### Compound 2-B,

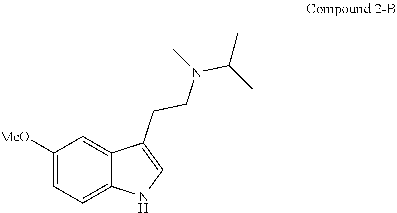 [SMILES: COc1ccc2[nH]cc(CCN(C)C(C)C)c2c1]

### Compound 2-C,

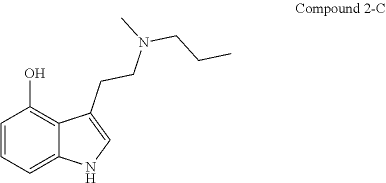 [SMILES: CCCN(C)CCc1c[nH]c2cccc(O)c12]

### Compound 2-D,

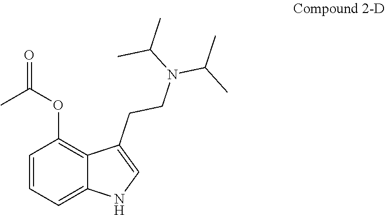 [SMILES: CC(=O)Oc1cccc2[nH]cc(CCN(C(C)C)C(C)C)c12]

### Compound 2-E

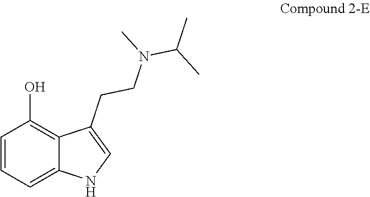 [SMILES: CC(C)N(C)CCc1c[nH]c2cccc(O)c12]

or a pharmaceutically acceptable salt, solvate, or hydrate thereof. In some embodiments, the tryptamine is Compound 2 or a pharmaceutically acceptable salt, solvate, or hydrate thereof. In some embodiments, the tryptamine is Compound 2-A or a pharmaceutically acceptable salt, solvate, or hydrate thereof. In some embodiments, the tryptamine is Compound 2-B or a pharmaceutically acceptable salt, solvate, or hydrate thereof. In some embodiments, the tryptamine is Compound 2-C or a pharmaceutically acceptable salt, solvate, or hydrate thereof. In some embodiments, the tryptamine is Compound 2-D or a pharmaceutically acceptable salt, solvate, or hydrate thereof. In some embodiments, the tryptamine is Compound 2-E or a pharmaceutically acceptable salt, solvate, or hydrate thereof. In some embodiments, the tryptamine is Compound 2-F or a pharmaceutically acceptable salt, solvate, or hydrate thereof.

In some embodiments, at least one of said two or more compounds comprises Compound 2-F

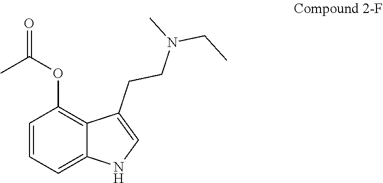 [SMILES: CCN(C)CCc1c[nH]c2cccc(OC(C)=O)c12]

or a pharmaceutically acceptable salt, solvate, or hydrate thereof.

In another aspect, provided herein is a combination comprising one or more compounds selected from:

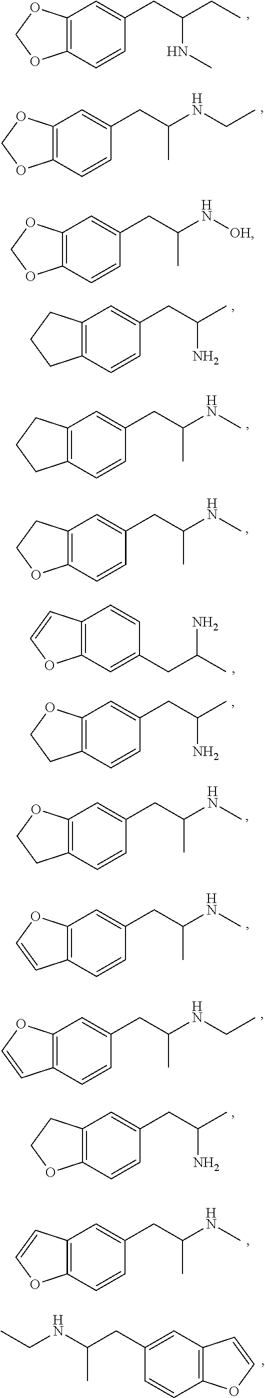

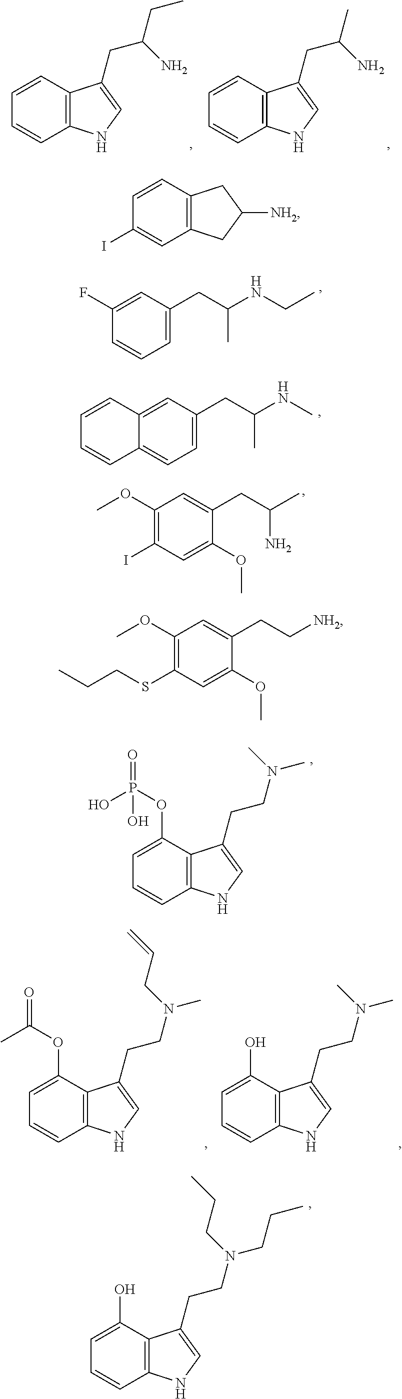

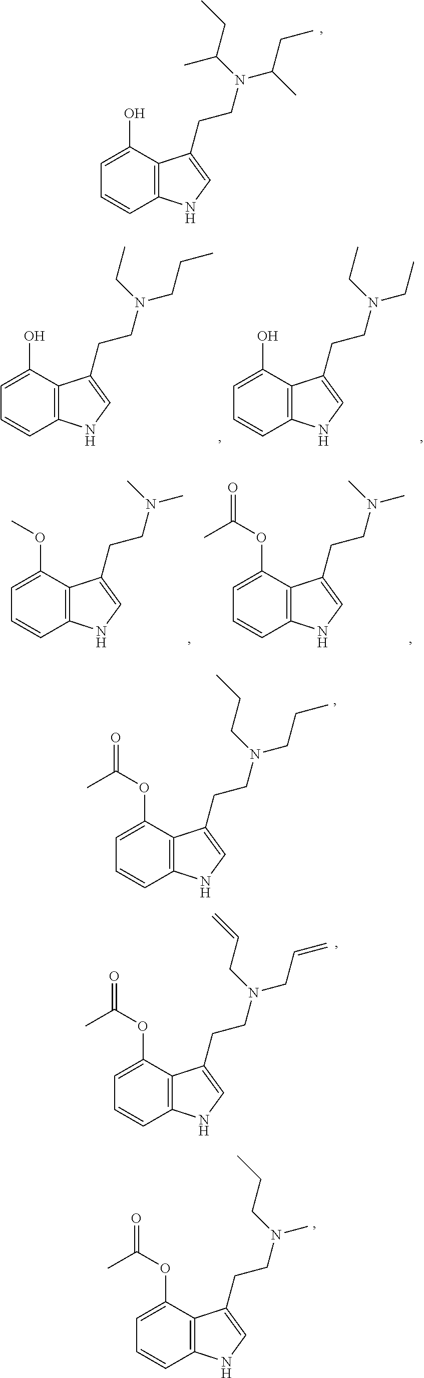

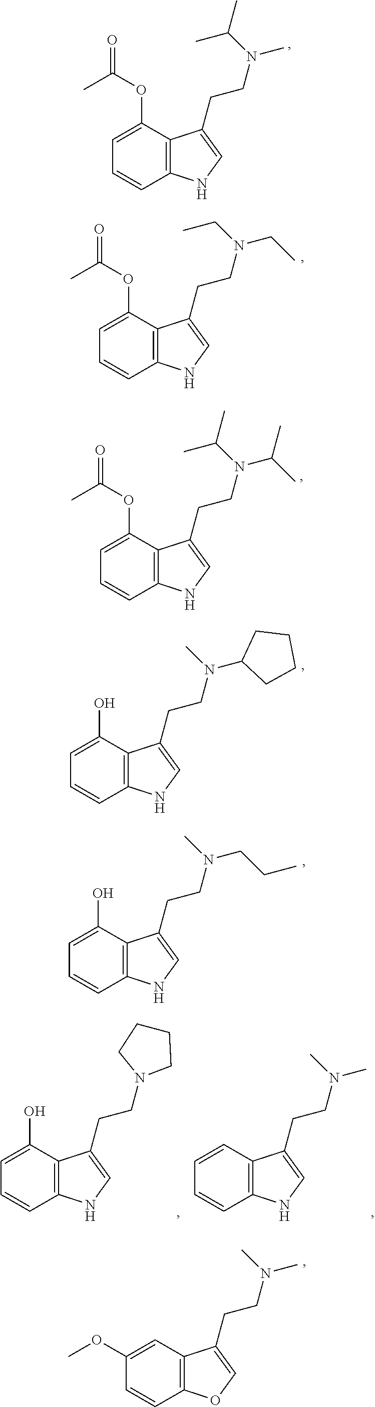

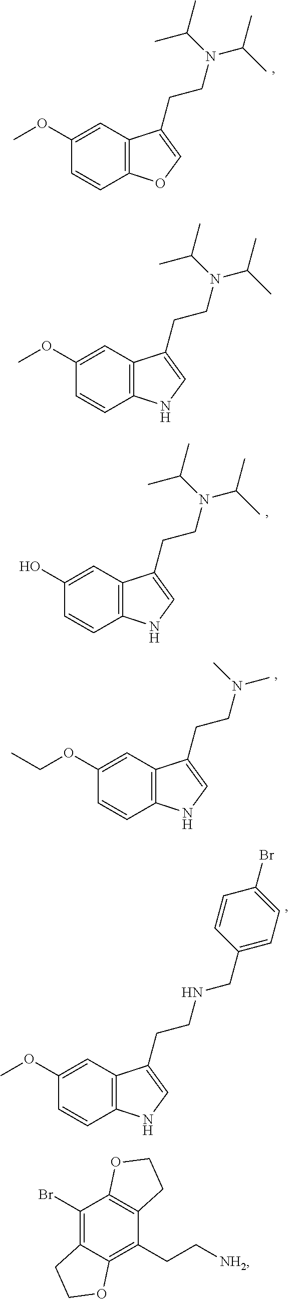

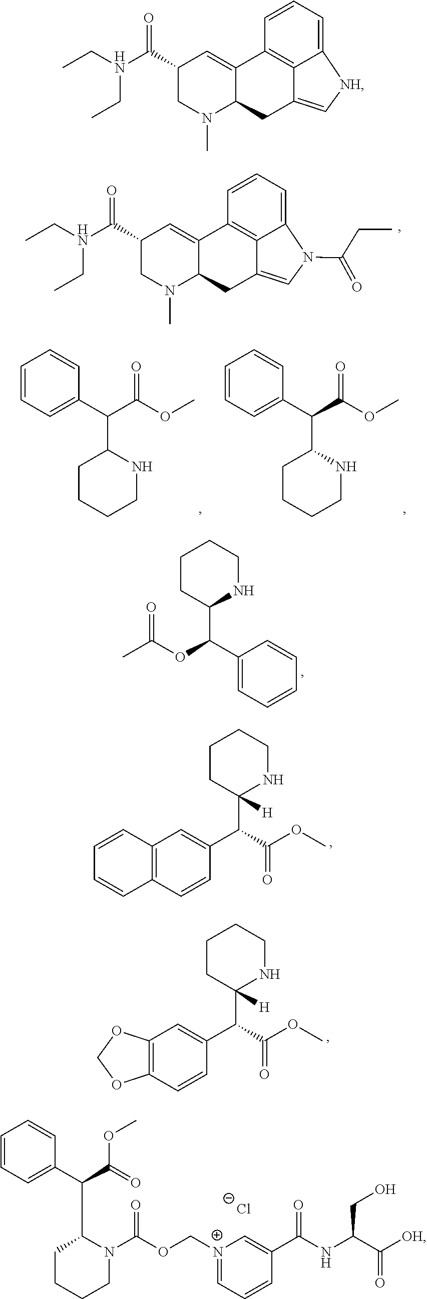

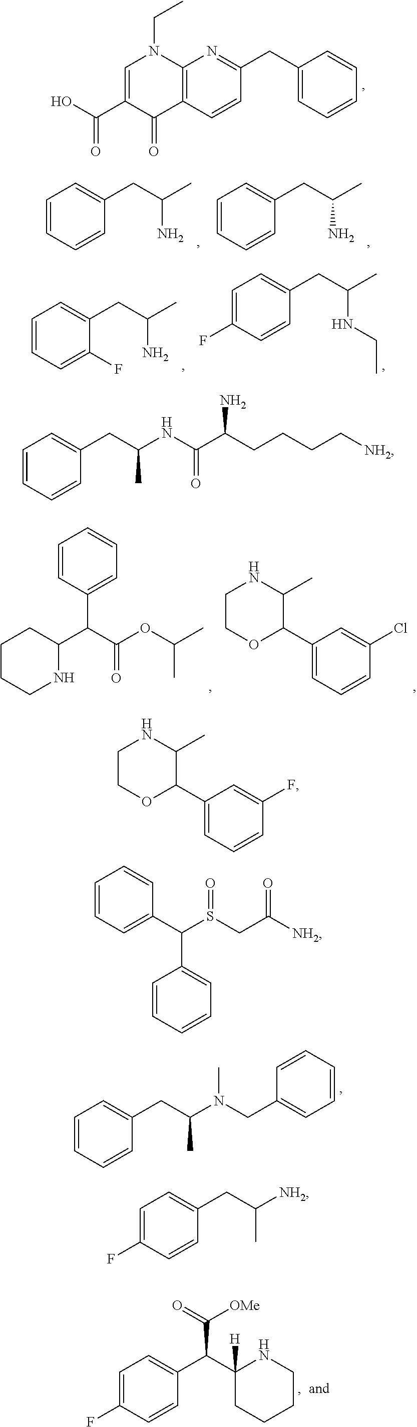

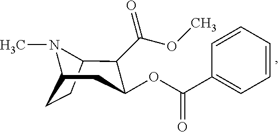

or pharmaceutically acceptable salts, solvates, or hydrates thereof.

In some embodiments, the compound, or a pharmaceutically acceptable salt, solvate, or hydrate thereof, is present in the combination at about 1 mg to about 100 mg. In some embodiments, the compound or a pharmaceutically acceptable salt, solvate, or hydrate thereof, is present in the combination at about 1 mg to about 10 mg. In some embodiments, the compound, or a pharmaceutically acceptable salt, solvate, or hydrate thereof, is present in the combination at about 10 mg to about 20 mg. In some embodiments, the compound, or a pharmaceutically acceptable salt, solvate, or hydrate thereof, is present in the combination at about 20 mg to about 30 mg. In some embodiments, the compound, or a pharmaceutically acceptable salt, solvate, or hydrate thereof, is present in the combination at about 30 mg to about 40 mg. In some embodiments, the compound, or a pharmaceutically acceptable salt, solvate, or hydrate thereof, is present in the combination at about 40 mg to about 50 mg. In some embodiments, the compound, or a pharmaceutically acceptable salt, solvate, or hydrate thereof, is present in the combination at about 50 mg to about 60 mg. In some embodiments, the compound, or a pharmaceutically acceptable salt, solvate, or hydrate thereof, is present in the combination at about 60 mg to about 70 mg. In some embodiments, the compound, or a pharmaceutically acceptable salt, solvate, or hydrate thereof, is present in the combination at about 70 mg to about 80 mg. In some embodiments, the compound, or a pharmaceutically acceptable salt, solvate, or hydrate thereof, is present in the combination at about 80 mg to about 90 mg. In some embodiments, the compound, or a pharmaceutically acceptable salt, solvate, or hydrate thereof, is present in the combination at about 90 mg to about 100 mg. In some embodiments, the compound, or a pharmaceutically acceptable salt, solvate, or hydrate thereof, is present in the combination at about 1 mg to about 5 mg. In some embodiments, the compound, or a pharmaceutically acceptable salt, solvate, or hydrate thereof, is present in the combination at about 5 mg to about 10 mg. In some embodiments, the compound, or a pharmaceutically acceptable salt, solvate, or hydrate thereof, is present in the combination at about 10 mg to about 15 mg. In some embodiments, the compound, or a pharmaceutically acceptable salt, solvate, or hydrate thereof, is present in the combination at about 15 mg to about 20 mg. In some embodiments, the compound, or a pharmaceutically acceptable salt, solvate, or hydrate thereof, is present in the combination at about 20 mg to about 25 mg. In some embodiments, the compound, or a pharmaceutically acceptable salt, solvate, or hydrate thereof, is present in the combination at about 25 mg to about 30 mg. In some embodiments, the compound, or a pharmaceutically acceptable salt, solvate, or hydrate thereof, is present in the combination at about 30 mg to about 35 mg. In some embodiments, the compound, or a pharmaceutically acceptable salt, solvate, or hydrate thereof, is present in the combination at about 35 mg to about 40 mg. In some embodiments, the compound, or a pharmaceutically acceptable salt, solvate, or hydrate thereof, is present in the combination at about 40 mg to about 45 mg. In some embodiments, the compound, or a pharmaceutically acceptable salt, solvate, or hydrate thereof, is present in the combination at about 45 mg to about 50 mg. In some embodiments, the compound, or a pharmaceutically acceptable salt, solvate, or hydrate thereof, is present in the combination at about 50 mg to about 55 mg. In some embodiments, the compound, or a pharmaceutically acceptable salt, solvate, or hydrate thereof, is present in the combination at about 55 mg to about 60 mg. In some embodiments, the compound, or a pharmaceutically acceptable salt, solvate, or hydrate thereof, is present in the combination at about 60 mg to about 65 mg. In some embodiments, the compound, or a pharmaceutically acceptable salt, solvate, or hydrate thereof, is present in the combination at about 65 mg to about 70 mg. In some embodiments, the compound, or a pharmaceutically acceptable salt, solvate, or hydrate thereof, is present in the combination at about 70 mg to about 75 mg. In some embodiments, the compound, or a pharmaceutically acceptable salt, solvate, or hydrate thereof, is present in the combination at about 75 mg to about 80 mg. In some embodiments, the compound, or a pharmaceutically acceptable salt, solvate, or hydrate thereof, is present in the combination at about 80 mg to about 85 mg. In some embodiments, the compound, or a pharmaceutically acceptable salt, solvate, or hydrate thereof, is present in the combination at about 85 mg to about 90 mg. In some embodiments, the compound, or a pharmaceutically acceptable salt, solvate, or hydrate thereof, is present in the combination at about 90 mg to about 95 mg. In some embodiments, the compound, or a pharmaceutically acceptable salt, solvate, or hydrate thereof, is present in the combination at about 95 mg to about 100 mg. In some embodiments, the compound, or a pharmaceutically acceptable salt, solvate, or hydrate thereof, is present in the combination at about 1 mg. In some embodiments, the compound or a pharmaceutically acceptable salt, solvate, or hydrate thereof, is present in the combination at about 2 mg. In some embodiments, the compound, or a pharmaceutically acceptable salt, solvate, or hydrate thereof, is present in the combination at about 3 mg. In some embodiments, the compound, or a pharmaceutically acceptable salt, solvate, or hydrate thereof, is present in the combination at about 4 mg. In some embodiments, the compound, or a pharmaceutically acceptable salt, solvate, or hydrate thereof, is present in the combination at about 5 mg. In some embodiments, the compound, or a pharmaceutically acceptable salt, solvate, or hydrate thereof, is present in the combination at about 6 mg. In some embodiments, the compound, or a pharmaceutically acceptable salt, solvate, or hydrate thereof, is present in the combination at about 7 mg. In some embodiments, the compound, or a pharmaceutically acceptable salt, solvate, or hydrate thereof, is present in the combination at about 8 mg. In some embodiments, the compound, or a pharmaceutically acceptable salt, solvate, or hydrate thereof, is present in the combination at about 9 mg. In some embodiments, the compound, or a pharmaceutically acceptable salt, solvate, or hydrate thereof, is present in the combination at about 10 mg. In some embodiments, the compound, or a pharmaceutically acceptable salt, solvate, or hydrate thereof, is present in the combination at about 11 mg. In some embodiments, the compound, or a pharmaceutically acceptable salt, solvate, or hydrate thereof, is present in the combination at about 12 mg. In some embodiments, the compound, or a pharmaceutically acceptable salt, solvate, or hydrate thereof, is present in the combination at about 13 mg. In some embodiments, the compound, or a pharmaceutically acceptable salt, solvate, or hydrate thereof, is present in the combination at about 14 mg. In some embodiments, the compound, or a pharmaceutically acceptable salt, solvate, or hydrate thereof, is present in the combination at about 15 mg. In some embodiments, the compound, or a pharmaceutically acceptable salt, solvate, or hydrate thereof, is present in the combination at about 16 mg. In some embodiments, the compound, or a pharmaceutically acceptable salt, solvate, or hydrate thereof, is present in the combination at about 17 mg. In some embodiments, the compound, or a pharmaceutically acceptable salt, solvate, or hydrate thereof, is present in the combination at about 18 mg. In some embodiments, the compound, or a pharmaceutically acceptable salt, solvate, or hydrate thereof, is present in the combination at about 19 mg. In some embodiments, the compound, or a pharmaceutically acceptable salt, solvate, or hydrate thereof, is present in the combination at about 20 mg. In some embodiments, the compound, or a pharmaceutically acceptable salt, solvate, or hydrate thereof, is present in the combination at about 21 mg. In some embodiments, the compound, or a pharmaceutically acceptable salt, solvate, or hydrate thereof, is present in the combination at about 22 mg. In some embodiments, the compound, or a pharmaceutically acceptable salt, solvate, or hydrate thereof, is present in the combination at about 23 mg. In some embodiments, the compound, or a pharmaceutically acceptable salt, solvate, or hydrate thereof, is present in the combination at about 24 mg. In some embodiments, the compound, or a pharmaceutically acceptable salt, solvate, or hydrate thereof, is present in the combination at about 25 mg. In some embodiments, the compound, or a pharmaceutically acceptable salt, solvate, or hydrate thereof, is present in the combination at about 30 mg. In some embodiments, the compound, or a pharmaceutically acceptable salt, solvate, or hydrate thereof, is present in the combination at about 35 mg. In some embodiments, the compound, or a pharmaceutically acceptable salt, solvate, or hydrate thereof, is present in the combination at about 40 mg. In some embodiments, the compound, or a pharmaceutically acceptable salt, solvate, or hydrate thereof, is present in the combination at about 45 mg. In some embodiments, the compound, or a pharmaceutically acceptable salt, solvate, or hydrate thereof, is present in the combination at about 50 mg. In some embodiments, the compound, or a pharmaceutically acceptable salt, solvate, or hydrate thereof, is present in the combination at about 55 mg. In some embodiments, the compound, or a pharmaceutically acceptable salt, solvate, or hydrate thereof, is present in the combination at about 60 mg. In some embodiments, the compound, or a pharmaceutically acceptable salt, solvate, or hydrate thereof, is present in the combination at about 65 mg. In some embodiments, the compound, or a pharmaceutically acceptable salt, solvate, or hydrate thereof, is present in the combination at about 70 mg. In some embodiments, the compound, or a pharmaceutically acceptable salt, solvate, or hydrate thereof, is present in the combination at about 75 mg. In some embodiments, the compound, or a pharmaceutically acceptable salt, solvate, or hydrate thereof, is present in the combination at about 80 mg. In some embodiments, the compound, or a pharmaceutically acceptable salt, solvate, or hydrate thereof, is present in the combination at about 85 mg. In some embodiments, the compound, or a pharmaceutically acceptable salt, solvate, or hydrate thereof, is present in the combination at about 90 mg. In some embodiments, the compound, or a pharmaceutically acceptable salt, solvate, or hydrate thereof, is present in the combination at about 95 mg. In some embodiments, the compound, or a pharmaceutically acceptable salt, solvate, or hydrate thereof, is present in the combination at about 100 mg.

### Exemplary Combinations

Exemplary combinations disclosed herein include about 1 mg to about 20 mg of the one or more compounds of Formula (I) or pharmaceutically acceptable salts, solvates, or hydrates thereof. In some embodiments, the combination comprises about 1 mg to about 20 mg of the one or more compounds of Formula (II) or pharmaceutically acceptable salts, solvates, or hydrates thereof. In some embodiments, the combination comprises about 1 mg to about 140 mg of the one or more compounds of Formula (III) or pharmaceutically acceptable salts, solvates, or hydrates thereof.

In some embodiments, the one or more compounds of Formula (III) are present at an amount that is 2-fold, 3-fold, 4-fold, 5-fold, 6-fold, 7-fold, or 8-fold greater than the amount of the one or more compounds of Formula (I), the one or more compounds of Formula (II), or both. In some embodiments, the one or more compounds of Formula (III) are present at an amount that is about 2-fold greater than the amount of the one or more compounds of Formula (I), the one or more compounds of Formula (II), or both to about 8-fold greater than the amount of the one or more compounds of Formula (I), the one or more compounds of Formula (II), or both. In some embodiments, the one or more compounds of Formula (III) are present at an amount that is about 2-fold greater than the amount of the one or more compounds of Formula (I), the one or more compounds of Formula (II), or both to about 3-fold greater than the amount of the one or more compounds of Formula (I), the one or more compounds of Formula (II), or both, about 2-fold greater than the amount of the one or more compounds of Formula (I), the one or more compounds of Formula (II), or both to about 4-fold greater than the amount of the one or more compounds of Formula (I), the one or more compounds of Formula (II), or both, about 2-fold greater than the amount of the one or more compounds of Formula (I), the one or more compounds of Formula (II), or both to about 5-fold greater than the amount of the one or more compounds of Formula (I), the one or more compounds of Formula (II), or both, about 2-fold greater than the amount of the one or more compounds of Formula (I), the one or more compounds of Formula (II), or both to about 6-fold greater than the amount of the one or more compounds of Formula (I), the one or more compounds of Formula (II), or both, about 2-fold greater than the amount of the one or more compounds of Formula (I), the one or more compounds of Formula (II), or both to ab out 7-fold greater than the amount of the one or more compounds of Formula (I), the one or more compounds of Formula (II), or both, about 2-fold greater than the amount of the one or more compounds of Formula (I), the one or more compounds of Formula (II), or both to about 8-fold greater than the amount of the one or more compounds of Formula (I), the one or more compounds of Formula (II), or both, about 3-fold greater than the amount of the one or more compounds of Formula (I), the one or more compounds of Formula (II), or both to about 4-fold greater than the amount of the one or more compounds of Formula (I), the one or more compounds of Formula (II), or both, about 3-fold greater than the amount of the one or more compounds of Formula (I), the one or more compounds of Formula (II), or both to about 5-fold greater than the amount of the one or more compounds of Formula (I), the one or more compounds of Formula (II), or both, about 3-fold greater than the amount of the one or more compounds of Formula (I), the one or more compounds of Formula (II), or both to about 6-fold greater than the amount of the one or more compounds of Formula (I), the one or more compounds of Formula (II), or both, about 3-fold greater than the amount of the one or more compounds of Formula (I), the one or more compounds of Formula (II), or both to about 7-fold greater than the amount of the one or more compounds of Formula (I), the one or more compounds of Formula (II), or both, about 3-fold greater than the amount of the one or more compounds of Formula (I), the one or more compounds of Formula (II), or both to about 8-fold greater than the amount of the one or more compounds of Formula (I), the one or more compounds of Formula (II), or both, about 4-fold greater than the amount of the one or more compounds of Formula (I), the one or more compounds of Formula (II), or both to about 5-fold greater than the amount of the one or more compounds of Formula (I), the one or more compounds of Formula (II), or both, about 4-fold greater than the amount of the one or more compounds of Formula (I), the one or more compounds of Formula (II), or both to about 6-fold greater than the amount of the one or more compounds of Formula (I), the one or more compounds of Formula (II), or b oth, about 4-fold greater than the amount of the one or more compounds of Formula (I), the one or more compounds of Formula (II), or both to about 7-fold greater than the amount of the one or more compounds of Formula (I), the one or more compounds of Formula (II), or both, about 4-fold greater than the amount of the one or more compounds of Formula (I), the one or more compounds of Formula (II), or both to about 8-fold greater than the amount of the one or more compounds of Formula (I), the one or more compounds of Formula (II), or both, about 5-fold greater than the amount of the one or more compounds of Formula (I), the one or more compounds of Formula (II), or both to about 6-fold greater than the amount of the one or more compounds of Formula (I), the one or more compounds of Formula (II), or both, about 5-fold greater than the amount of the one or more compounds of Formula (I), the one or more compounds of Formula (II), or both to about 7-fold greater than the amount of the one or more compounds of Formula (I), the one or more compounds of Formula (II), or both, about 5-fold greater than the amount of the one or more compounds of Formula (I), the one or more compounds of Formula (II), or both to about 8-fold greater than the amount of the one or more compounds of Formula (I), the one or more compounds of Formula (II), or both, about 6-fold greater than the amount of the one or more compounds of Formula (I), the one or more compounds of Formula (II), or both to about 7-fold greater than the amount of the one or more compounds of Formula (I), the one or more compounds of Formula (II), or both, about 6-fold greater than the amount of the one or more compounds of Formula (I), the one or more compounds of Formula (II), or both to about 8-fold greater than the amount of the one or more compounds of Formula (I), the one or more compounds of Formula (II), or both, or about 7-fold greater than the amount of the one or more compounds of Formula (I), the one or more compounds of Formula (II), or both to about 8-fold greater than the amount of the one or more compounds of Formula (I), the one or more compounds of Formula (II), or both. In some embodiments, the one or more compounds of Formula (III) are present at an amount that is about 2-fold greater than the amount of the one or more compounds of Formula (I), the one or more compounds of Formula (II), or both, about 3-fold greater than the amount of the one or more compounds of Formula (I), the one or more compounds of Formula (II), or both, about 4-fold greater than the amount of the one or more compounds of Formula (I), the one or more compounds of Formula (II), or both, about 5-fold greater than the amount of the one or more compounds of Formula (I), the one or more compounds of Formula (II), or b oth, about 6-fold greater than the amount of the one or more compounds of Formula (I), the one or more compounds of Formula (II), or both, about 7-fold greater than the amount of the one or more compounds of Formula (I), the one or more compounds of Formula (II), or both, or about 8-fold greater than the amount of the one or more compounds of Formula (I), the one or more compounds of Formula (II), or both. In some embodiments, the one or more compounds of Formula (III) are present at an amount that is at least about 2-fold greater than the amount of the one or more compounds of Formula (I), the one or more compounds of Formula (II), or both, about 3-fold greater than the amount of the one or more compounds of Formula (I), the one or more compounds of Formula (II), or both, about 4-fold greater than the amount of the one or more compounds of Formula (I), the one or more compounds of Formula (II), or both, about 5-fold greater than the amount of the one or more compounds of Formula (I), the one or more compounds of Formula (II), or both, about 6-fold greater than the amount of the one or more compounds of Formula (I), the one or more compounds of Formula (II), or both, or about 7-fold greater than the amount of the one or more compounds of Formula (I), the one or more compounds of Formula (II), or both. In some embodiments, the one or more compounds of Formula (III) are present at an amount that is at most about 3-fold greater than the amount of the one or more compounds of Formula (I), the one or more compounds of Formula (II), or both, about 4-fold greater than the amount of the one or more compounds of Formula (I), the one or more compounds of Formula (II), or both, about 5-fold greater than the amount of the one or more compounds of Formula (I), the one or more compounds of Formula (II), or both, about 6-fold greater than the amount of the one or more compounds of Formula (I), the one or more compounds of Formula (II), or both, about 7-fold greater than the amount of the one or more compounds of Formula (I), the one or more compounds of Formula (II), or both, or about 8-fold greater than the amount of the one or more compounds of Formula (I), the one or more compounds of Formula (II), or both. In some embodiments, the one or more compounds of Formula (III) are present at an amount that is 7-fold greater than the amount of the one or more compounds of Formula (I) and the one or more compounds of Formula (II).

In some embodiments, the combination comprises: 10 mg of the one or more compounds of Formula (I); 10 mg of the one or more compounds of Formula (II); and 70 mg of the one or more compounds of Formula (III). In some embodiments, the one or more compounds of Formula (I) is Compound 1. In some embodiments, the one or more compounds of Formula (II) is Compound 2. In some embodiments, the one or more compounds of Formula (III) is Compound 3.

Another aspect of the present disclosure is method for treating or alleviating a disease, a disorder, or a condition in a subject in need thereof, the method comprising administering to the subject: the combination of any one of the afore-mentioned embodiments, or pharmaceutically acceptable salts, solvates, or hydrates thereof. In some embodiments, the disease, the disorder, or the condition is selected from sexual abuse, mood disorders, phobia disorders, dissociative disorders, stress disorders, personality disorders, obsessive compulsive disorders, tourette's disorder, grief, autism, eating disorders, alcohol use disorder, conduct disorder, conversion disorder, and addiction. In some embodiments, the sexual abuse is selected from adult sexual disorder and child sexual disorder. In some embodiments, the mood disorder is selected from persistent depressive disorder or dysthymia, major depressive disorder, cyclothymic disorder, premenstrual dysphoric disorder, generalized anxiety disorder, and panic disorder. In some embodiments, the phobia disorder is selected from phobia of animal, phobia of blood-injection-injury, phobia of blood, phobia of injections or transfusions, phobia of injury, phobia of medical care, phobia of natural environment, and situational phobias. In some embodiments, the stress disorder is selected from acute stress disorder, posttraumatic stress disorder, unspecified trauma- and stressor-related disorder, nightmare disorder, adjustment disorder, reactive attachment disorder, and disinhibited attached disorder. In some embodiments, the personality disorder is selected from antisocial behavior, antisocial personality disorder, avoidant personality disorder, borderline personality disorder, narcissistic personality disorder, and obsessive-compulsive personality disorder. In some embodiments, the eating disorder is selected from avoidant/restrictive food intake disorder, binge-eating disorder, body dysmorphic disorder, bulimia nervosa, and anorexia nervosa. In some embodiments, the addiction is selected from tobacco use addiction, opioid use disorder, and alcohol use disorder.

The compounds disclosed herein, in some embodiments, are used in different enriched isotopic forms, e.g., enriched in the content of ²H, ³H, ¹¹C, ¹³C and/or ¹⁴C. Deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds disclosed herein, whether radioactive or not, are encompassed within the scope of this disclosure.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

The compounds disclosed herein also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, an d isolating its conversion products from the urine, blood or other biological samples.

### Pharmaceutical Formulations

A combination of the present disclosure is formulated in any suitable pharmaceutical formulation. A pharmaceutical formulation of the present disclosure typically contains an active ingredient (e.g., a combination disclosed herein), and one or more pharmaceutically acceptable excipients or carriers, including but not limited to: inert solid diluents and fillers, diluents, sterile aqueous solution and various organic solvents, permeation enhancers, solubilizers, and adjuvants. In some embodiments, the pharmaceutical acceptable carriers or excipients are selected from water, alcohol, glycerol, chitosan, alginate, chondroitin, Vitamin E, mineral oil, and dimethyl sulfoxide (DMSO).

Pharmaceutical formulations are provided in any suitable form, which is determined based on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of each combination administered will be dependent on the severity of the disorder or condition, the rate of administration, the disposition of the combination and the discretion of the prescribing physician In some embodiments, an effective dosage is provided in pulsed dosing (i.e., administration of the combination in consecutive days, followed by consecutive days of rest from administration).

In some embodiments, the disclosure provides a pharmaceutical composition for oral administration containing at least one combination disclosed herein and a pharmaceutical excipient suitable for oral administration. The composition is in the form of a solid, liquid, gel, semi-liquid, or semi-solid.

In some embodiments, this disclosure provides a solid pharmaceutical composition for oral administration containing: (i) a combination disclosed herein; and (ii) a pharmaceutical excipient suitable for oral administration. In some embodiments, each compound or agent is present in a therapeutically effective amount. In other embodiments, one or more compounds or agents is present in a sub-therapeutic amount, and the compounds or agents act synergistically to provide a therapeutically effective pharmaceutical composition.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered combination moistened with an inert liquid diluent.

In some embodiments, the disclosure provides a pharmaceutical composition for injection containing a combination disclosed herein and a pharmaceutical excipient suitable for injection. Components and amounts of agents in the composition are as described herein.

In certain embodiments, the forms in which the combination disclosed herein are incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, f or example, parabens, chlorobutanol, phenol, sorbic acid, thimerosal, and the like.

Sterile injectable solutions are prepared by incorporating the combination disclosed herein in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions may also be prepared from a combination described herein and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

The disclosure also provides kits. The kits may include a combination disclosed herein and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the combination disclosed herein and the agent are provided as separate compositions in separate containers within the kit. In some embodiments, the combination disclosed herein and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Methods of Use

Psychedelics entactogens have the capacity to create the conditions for greater love, connection, intimacy, unity, sensuality, and communication in individuals who are suffering from sexual dysfunction or social disconnection. Since high levels of dopamine, serotonin, and norepinephrine are released during the attraction phase of romantic love, each hormone has specific effects on transforming the way individuals feel when in love.

Drug-assisted therapy, such as a combination as described herein, has the potential to alter a couple's neurochemistry towards love, connection, passion, and belonging through the creation of a peak experience that helps them resolve conflict, build trust, enhance intimacy, increase desire, and strengthen bonds. Furthermore, a drug-assisted therapy, such as a combination described herein, provides a solution to conditions such as psychogenic erectile dysfunction, hypoactive sexual desire disorder, and other sexual dysfunctions rooted in relationship problems.

Accordingly, in one aspect, provided herein is a method for treating or alleviating a sexual disorder in a subject in need thereof, the method comprising administering to said subject a combination provided herein, or pharmaceutically acceptable salts, solvates, or hydrates thereof, a pharmaceutical composition provided herein, or pharmaceutically acceptable salts, solvates, or hydrates thereof, or an oral unit dosage form provided herein.

In some embodiments, said sexual disorder is selected from hypoactive sexual desire disorder, sexual arousal disorder, anorgasmia, vaginismus, dyspareunia, erectile dysfunction, diminished libido, and abnormal ejaculation. In some embodiments, said sexual disorder is hypoactive sexual desire disorder. In some embodiments, said sexual disorder is sexual arousal disorder. In some embodiments, said sexual disorder is anorgasmia. In some embodiments, said sexual disorder is vaginismus. In some embodiments, said sexual disorder is dyspareunia. In some embodiments, said sexual disorder is erectile dysfunction. In some embodiments, said sexual disorder is diminished libido. In some embodiments, said sexual disorder is abnormal ejaculation.

In another aspect, provided herein is a method for enhancing sexual drive in a subject, the method comprising administering to said subject a combination provided herein, or pharmaceutically acceptable salts, solvates, or hydrates thereof, a pharmaceutical composition provided herein, or pharmaceutically acceptable salts, solvates, or hydrates thereof, or an oral unit dosage form provided herein.

In some embodiments, the subject is a subject with a penis. In some embodiments, the subject is a subject with a vagina. In some embodiments, the subject is a man. In some embodiments, the subject is a woman.

In another aspect, provided herein is a method of treating or alleviating a disease, a disorder, or a condition, in a subject in need thereof, the method comprising administering to said subject a combination provided herein, or pharmaceutically acceptable salts, solvates, or hydrates thereof, a pharmaceutical composition provided herein, or the oral unit dosage form provided herein.

In some embodiments, the disease, the disorder, or the condition is selected from sexual abuse, mood disorders, phobia disorders, dissociative disorders, stress disorders, personality disorders, obsessive compulsive disorders, tourette's disorder, grief, autism, eating disorders, alcohol use disorder, conduct disorder, conversion disorder, and addiction.

In some embodiments, the disease, the disorder, or the condition is sexual abuse. In some embodiments, the sexual abuse is selected from adult sexual disorder and child sexual disorder. In some embodiments, the adult sexual abuse is by non-spouse or non-partner. In some embodiments, the adult sexual abuse by non-spouse or non-partner is during the initial encounter. In some embodiments, the adult sexual abuse by non-spouse or non-partner is during the subsequent encounter. In some embodiments, the adult sexual abuse is confirmed. In some embodiments, the adult sexual abuse is suspected. In some embodiments, the child sexual abuse is during the initial encounter. In some embodiments, the child sexual abuse is during the subsequent encounter. In some embodiments, the child sexual abuse is confirmed. In some embodiments, the child sexual abuse is suspected. In some embodiments, the child sexual abuse is by a parent. In some embodiments, the child sexual abuse is by a non-parent. In some embodiments, the sexual abuse is caused by other circumstances.

In some embodiments, the disease, the disorder, or the condition is mood disorder. In some embodiments, the mood disorder is selected from persistent depressive disorder or dysthymia, major depressive disorder, cyclothymic disorder, premenstrual dysphoric disorder, generalized anxiety disorder, and panic disorder. In some embodiments, the major depressive disorder is recurrent. In some embodiments, the recurrent major depressive disorder is mild. In some embodiments, the recurrent major depressive disorder is moderate. In some embodiments, the recurrent major depressive disorder is severe. In some embodiments, the recurrent major depressive disorder is with psychotic features. In some embodiments, the major depressive disorder is a single episode. In some embodiments, the single major depressive disorder episode is mild. In some embodiments, the single major depressive disorder episode is moderate. In some embodiments, the single major depressive disorder episode is severe.

In some embodiments, the disease, the disorder, or the condition is phobia disorder. In some embodiments, the phobia disorder is selected from phobia of animal, phobia of blood-injection-injury, phobia of blood, phobia of injections or transfusions, phobia of injury, phobia of medical care, phobia of natural environment, and situational phobias.

In some embodiments, the disease, the disorder, or the condition is dissociative disorder. In some embodiments, the dissociative disorder is dissociative amnesia. In some embodiments, the dissociative disorder is dissociative identity disorder.

In some embodiments, the disease, the disorder, or the condition is stress disorder. In some embodiments, the stress disorder is selected from acute stress disorder, posttraumatic stress disorder, unspecified trauma- and stressor-related disorder, nightmare disorder, adjustment disorder, reactive attachment disorder, and disinhibited attached disorder. In some embodiments, the stress disorder is an adjustment disorder. In some embodiments, the adjustment disorder is unspecified. In some embodiments, the adjustment disorder is with anxiety. In some embodiments, the adjustment disorder is with depressed mood. In some embodiments, the adjustment disorder is with disturbance of conduct. In some embodiments, the adjustment disorder is with mixed anxiety and depressed mood. In some embodiments, the adjustment disorder is with mixed disturbance of emotions and conduct.

In some embodiments, the disease, the disorder, or the condition is personality disorder. In some embodiments, the personality disorder is selected from antisocial behavior, antisocial personality disorder, avoidant personality disorder, borderline personality disorder, narcissistic personality disorder, and obsessive-compulsive personality disorder.

In some embodiments, the disease, the disorder, or the condition is obsessive compulsive disorder.

In some embodiments, the disease, the disorder, or the condition is Tourette's disorder.

In some embodiments, the disease, the disorder, or the condition is grief. In some embodiments, the grief is uncomplicated bereavement. In some embodiments, the grief is persistent complex bereavement disorder.

In some embodiments, the disease, the disorder, or the condition is autism.

In some embodiments, the disease, the disorder, or the condition is eating disorder. In some embodiments, the eating disorder is selected from avoidant/restrictive food intake disorder, binge-eating disorder, body dysmorphic disorder, bulimia nervosa, and anorexia nervosa. In some embodiments, the anorexia nervosa is binge-eating or purging type. In some embodiments, the anorexia nervosa is restricting type.

In some embodiments, the disease, the disorder, or the condition is alcohol use disorder. In some embodiments, the alcohol use disorder is mild. In some embodiments, the alcohol use disorder is moderate. In some embodiments, the alcohol use disorder is severe.

In some embodiments, the disease, the disorder, or the condition is conduct disorder. In some embodiments, the conduct disorder is adolescent-onset type. In some embodiments, the conduct disorder is childhood-onset type. In some embodiments, the conduct disorder is unspecified.

In some embodiments, the disease, the disorder, or the condition is conversion disorder or functional neurological symptom disorder. In some embodiments, the conversion disorder is with abnormal movement. In some embodiments, the conversion disorder is with anesthesia or sensory loss. In some embodiments, the conversion disorder is with attacks or seizures. In some embodiments, the conversion disorder is with mixed symptoms. In some embodiments, the conversion disorder is with special sensory symptoms. In some embodiments, the conversion disorder is with speech symptoms. In some embodiments, the conversion disorder is with swallowing symptoms. In some embodiments, the conversion disorder is with weakness or paralysis.

In some embodiments, the disease, the disorder, or the condition is addiction. In some embodiments, the addiction is selected from tobacco use addiction, opioid use disorder, and alcohol use disorder. In some embodiments, the tobacco use disorder is mild. In some embodiments, the tobacco use disorder is moderate. In some embodiments, the tobacco use disorder is severe. In some embodiments, the opioid use disorder is mild. In some embodiments, the opioid use disorder is moderate. In some embodiments, the opioid use disorder is severe. In some embodiments, the alcohol use disorder is mild. In some embodiments, the alcohol use disorder is moderate. In some embodiments, the alcohol use disorder is severe.

As used herein, a therapeutically effective amount of a combination disclosed herein refers to an amount sufficient to affect the intended application, including but not limited to, disease treatment, as defined herein. Also contemplated in the subject methods is the use of a sub-therapeutic amount of a combination disclosed herein for treating an intended disease condition.

The amount of the combination disclosed herein administered will vary depending upon the subject and disease condition being treated, e.g., the weight and age of the subject, the severity of the disease condition, the manner of administration and the like, which can readily be determined by one of ordinary skill in the art.

In some embodiments, the therapeutic efficacy is measured based on an effect of treating a disease, a disorder, or a condition as described herein. In some embodiments, therapeutic efficacy is measured based on an effect of treating a sexual disorder, abuse, a mood disorder, a phobia disorder, a dissociative disorder, a stress disorder, a personality disorders, an obsessive-compulsive disorders Tourette's disorder, grief, autism, an eating disorder, an alcohol use disorder, a conduct disorder, a conversion disorder, or an addiction. In general, therapeutic efficacy of the methods and compositions disclosed herein, with regard to the treatment of a sexual disorder abuse, a mood disorder, a phobia disorder, a dissociative disorder, a stress disorder, a personality disorders, an obsessive compulsive disorders Tourette's disorder, grief, autism, an eating disorder, an alcohol use disorder, a conduct disorder, a conversion disorder, or an addiction, may be measured by the degree to which the methods and compositions promote an increase in sexual satisfaction. Several parameters to be considered in the determination of therapeutic efficacy are discussed herein. The proper combination of parameters for a particular situation can be established by the clinician. The progress of the method disclosed herein in treating a sexual disorder abuse, a mood disorder, a phobia disorder, a dissociative disorder, a stress disorder, a personality disorders, an obsessive-compulsive disorders Tourette's disorder, grief, autism, an eating disorder, an alcohol use disorder, a conduct disorder, a conversion disorder, or an addiction can be ascertained using any suitable method, such as those methods currently used in the clinic to determine sexual satisfaction for a sexual disorder or dysfunction. Exemplary efficacy parameters used to evaluate the treatment of a sexual disorder, sexual dysfunction, or a related disease, disorder, or condition by the method and compositions disclosed herein include, but are not limited to, level of sexual desire, level of sexual arousal, level of distress, reduction of distress, frequency of sexual desire, frequency of sexual arousal, frequency of sexual activity, frequency of orgasm, frequency of successful vaginal penetration, ease of vaginal penetration, level of vaginal lubrication, level of pain during sexual intercourse, frequency of erections, quality of erections, timing of ejaculation, overall sexual satisfaction, or frequency of distress. In some embodiments, the compositions and methods disclosed herein result in greater feelings of connection, romantic love, or pair bonding with sexual partners.

The treatment of a sexual disorder, abuse, a mood disorder, a phobia disorder, a dissociative disorder, a stress disorder, a personality disorders, an obsessive compulsive disorders Tourette's disorder, grief, autism, an eating disorder, an alcohol use disorder, a conduct disorder, a conversion disorder, and an addiction in a human patient desirably is evidenced by one or more (in any combination) of the foregoing results, although alternative or additional results of the referenced tests and/or other tests can evidence treatment efficacy.

In some embodiments, administration of a combination disclosed herein provides improved therapeutic efficacy. Improved efficacy may be measured using any method known in the art, including but not limited to those described herein. In some embodiments, the improved therapeutic efficacy is an improvement of at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000% or more, using an appropriate measure (e.g. level of sexual desire, level of sexual arousal, frequency of sexual desire, frequency of sexual arousal, frequency of sexual activity, frequency of orgasm, frequency of successful vaginal penetration, ease of vaginal penetration, level of vaginal lubrication, level of pain during sexual intercourse, frequency of erections, quality of erections, timing of ejaculation, and overall sexual satisfaction). Improved efficacy may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold or more, using an appropriate measure (e.g. level of sexual desire, level of sexual arousal, frequency of sexual desire, frequency of sexual arousal, frequency of sexual activity, frequency of orgasm, frequency of successful vaginal penetration, ease of vaginal penetration, level of vaginal lubrication, level of pain during sexual intercourse, frequency of erections, quality of erections, timing of ejaculation, and overall sexual satisfaction).

A subject being treated with a combination disclosed herein may be monitored to determine the effectiveness of treatment, and the treatment regimen may be adjusted based on the subject's physiological response to treatment. For example, if a clinical indication is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with a combination disclosed herein is discontinued if a clinical indication is above or below a threshold, such as in a lack of response or an adverse reaction. The biological effect may be a change in any of a variety of physiological indicators.

### Combination Therapy

In some embodiments, disclosed herein are methods for further combination therapies in which, in addition to a combination described herein, one or more second agents known to modulate other pathways, or other components of the same pathway, or even overlapping sets of target proteins is used. In one aspect, such therapy includes but is not limited to the combination of the composition comprising a combination described herein with one or more therapeutic agents to provide, where desired, a synergistic or additive therapeutic effect.

In some embodiments, disclosed herein is a method for using the combinations described herein or pharmaceutical composition in combination with other treatment approaches, such as sex therapy and couple's counseling.

### Biochemistry

The aforementioned methods of use and combination therapies are modulated by the effect the combinations disclosed herein (e.g. the exemplary combinations) have on one or more of serotonin receptors (e.g. 5ht2a, 5ht2c, and 5ht1a); dopamine receptors (e.g. D1, D2, D3); adrenergic receptors (e.g. α1, α2); serotonin transporter (SERT); norepinephrine transporter (NET); dopamine transporter (DAT); histamine receptors (e.g. H1); and Monoamine transporters (e.g. VMAT1, VMAT2). In some embodiments, the combinations disclosed herein have excitatory (and in cases of multiplex receptor activity observation, also inhibitory) effects on the one or more of serotonin receptors; dopamine receptors; adrenergic receptors; serotonin transporter; norepinephrine transporter; dopamine transporter; histamine receptors; and Monoamine transporters. In some embodiments, the combination comprises Compound 1 and Compound 2. In some embodiments, the combination comprises Compound 1, Compound 2, and Compound 3. In some embodiments, Compound 3 is present in the combination at 2-fold, 3-fold, 4-fold, 5-fold, 6-fold, 7-fold, or 8-fold the amount present in the combination of Compound 1, Compound 2, or both. In some embodiments, Compound 3 is present in the combination at 7-fold the amount present in the combination of Compound 1, Compound 2, or both. In some embodiments, Compound 3 is present in the combination at 7-fold the amount present in the combination of Compound 1 and Compound 2.

In some embodiments, at least a subset of the effects of one or more of serotonin receptors; dopamine receptors; adrenergic receptors; serotonin transporter; norepinephrine transporter; dopamine transporter; histamine receptors; and Monoamine transporters by the combinations disclosed herein resemble the activity pattern of the same receptor in response to MDMA administration. In these embodiments, the neurotoxic effects of administration of the combinations disclosed herein are significantly less than the neurotoxic effects of administration of MDMA.

### EXAMPLES

### Example 1: Phase I Clinical Trial for Treatment of Sexual Disorders

100 sexually active subjects suffering from one or more sexual disorders (50 subjects with penises and 50 subjects with vaginas) are enrolled in a Phase I clinical study to determine the safety and efficacy of a combination of Compounds 1, 2, and 3. A fixed-dose combination of Compounds 1, 2, and 3 is administered in a controlled clinical setting once per week for a period of 8 weeks. Based upon weekly self-reporting, the frequency of sexual activity, the frequency of successful vaginal penetration, the frequency of orgasm, reduction of distress, and overall sexual satisfaction of each patient is tracked over time. Patients are given a physical evaluation on a weekly basis to determine the presence of adverse events.

Groups receiving the combination of Compounds 1, 2, and 3, reported significantly more frequent sexual activity and overall sexual satisfaction in comparison to control groups.

### Example 2: Supported Medication Experience and Survey Results

A survey was conducted on 19 adults (13 male and 6 female) of age 21 or older on supported medication for 13 months in Costa Rica.

Over a 5-month period, individuals were administered a combination of 6 mg of 4-acetoxy-N-methyl-N-ethyltryptamine (metacetin or 4-AcO-MET), 68 mg of 1-(benzofuran-5-yl)-N-methylpropan-2-amine (5-MAPB), and 12 mg of 2-fluoromethamphetamine (2-FMA).

During March of 2018 individuals were administered a combination of 6.5 mg of 4-AcO-MET, 72 mg 5-MAPB, and 10.5 mg of 4-fluoromfetamphetamine (4-FMA).

The locations of where individuals consumed the medicine varied. 11 individuals took the medicine at their homes or at a friend's home, one person took their dose on a beach, 3 people took their medicines at their home and left to visit a music venue, 2 people took their dose s at a music venue, 1 person took their dose at a festival, and 1 person took their dose on a cruise ship.

### Results

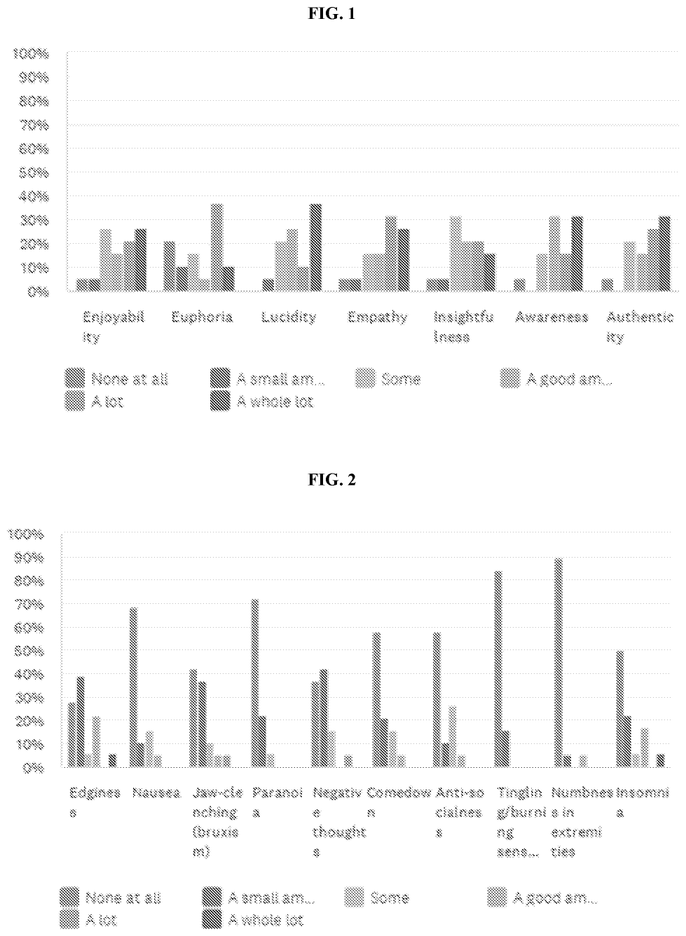

FIG. 1 shows a bar graph of the positive feelings that were experienced by the participants. Table 1 below further summarizes the data shown in FIG. 1.

TABLE 1
Positive Feelings Experienced by the Participants

| | None | A small | | A good | | A whole | | Weighted |
|---|---|---|---|---|---|---|---|---|
| | at all | amount | Some | amount | A lot | lot | Total | average |
| Enjoyability | 5.26% | 5.26% | 26.32% | 15.79% | 21.05% | 26.32% | 19 | 3.21 |
| | 1 | 1 | 5 | 3 | 4 | 5 | | |
| Euphoria | 21.05% | 10.53% | 15.79% | 5.26% | 36.84% | 10.53% | 19 | 2.58 |
| | 4 | 2 | 3 | 1 | 7 | 2 | | |
| Lucidity | 0.00% | 5.26% | 21.05% | 26.32% | 10.53% | 36.84% | 19 | 3.53 |
| | 0 | 1 | 4 | 5 | 2 | 7 | | |
| Empathy | 5.26% | 5.26% | 15.79% | 15.79% | 31.58% | 26.32% | 19 | 3.42 |
| | 1 | 1 | 3 | 3 | 6 | 5 | | |
| Insightfulness | 5.26% | 5.26% | 31.58% | 21.05% | 21.05% | 15.79% | 19 | 2.95 |
| | 1 | 1 | 6 | 4 | 4 | 3 | | |
| Awareness | 5.26% | 0.00% | 15.79% | 31.58% | 15.79% | 31.58% | 19 | 3.47 |
| | 1 | 0 | 3 | 6 | 3 | 6 | | |
| Authenticity | 5.26% | 0.00% | 21.05% | 15.79% | 26.32% | 31.58% | 19 | 3.53 |
| | 1 | 0 | 4 | 3 | 5 | 6 | | |

FIG. 2 shows a bar graph of the negative feelings that were experienced by the participants. Table 2 below further summarizes the data shown in FIG. 2.

TABLE 2
Negative Feelings Experienced by the Participants

| | None | A small | | A good | | A whole | | Weighted |
|---|---|---|---|---|---|---|---|---|
| | at all | amount | Some | amount | A lot | lot | Total | average |
| Edginess | 26.32% | 36.84% | 5.26% | 21.05% | 0.00% | 5.26% | 19 | 1.44 |
| | 5 | 7 | 1 | 4 | 0 | 1 | | |
| Nausea | 68.42% | 10.53% | 15.79% | 5.26% | 0.00% | 0.00% | 19 | 0.58 |
| | 13 | 2 | 3 | 1 | 0 | 0 | | |
| Jaw-clenching | 42.11% | 36.84% | 10.53% | 5.26% | 5.26% | 0.00% | 19 | 0.95 |
| (bruxism) | 8 | 7 | 2 | 1 | 1 | 0 | | |
| Paranoia | 72.22% | 22.22% | 5.26% | 0.00% | 0.00% | 0.00% | 19 | 0.33 |
| | 13 | 4 | 1 | 0 | 0 | 0 | | |
| Negative | 36.84% | 42.11% | 15.79% | 0.00% | 5.26% | 0.00% | 19 | 0.95 |
| thoughts | 7 | 8 | 3 | 0 | 1 | 0 | | |
| Comedown | 57.89% | 21.05% | 15.79% | 5.26% | 0.00% | 0.00% | 19 | 0.68 |
| | 11 | 4 | 3 | 1 | 0 | 0 | | |
| Anti- | 57.89% | 10.53% | 26.32% | 5.26% | 0.00% | 0.00% | 19 | 0.79 |
| socialness | 11 | 2 | 5 | 1 | 0 | 0 | | |
| Tingling/Burning | 84.21% | 15.79% | 0.00% | 0.00% | 0.00% | 0.00% | 19 | 0.16 |
| sensation | 16 | 3 | 0 | 0 | 0 | 0 | | |
| Numbness in | 89.47% | 5.26% | 0.00% | 5.26% | 0.00% | 0.00% | 19 | 0.21 |
| extremities | 17 | 1 | 0 | 1 | 0 | 0 | | |
| Insomnia | 50.00% | 22.22% | 5.56% | 16.67% | 0.00% | 5.56% | 18 | 1.11 |
| | 9 | 4 | 1 | 3 | 0 | 1 | | |

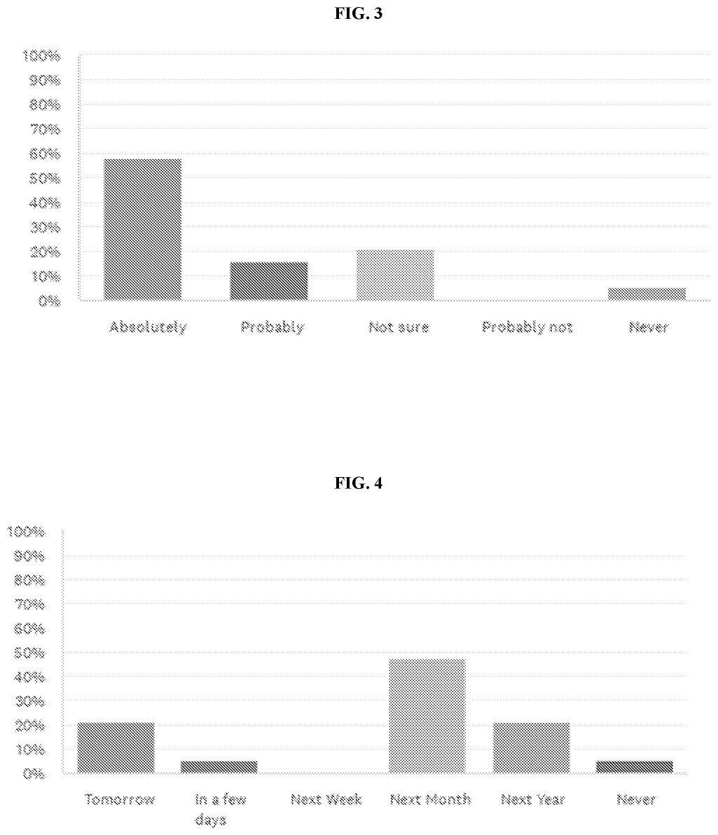

FIG. 3 shows the survey results on the question “would the participant like to participate in the same experience again”. Table 3 below further summarizes the data shown in FIG. 3.

TABLE 3
Answers to whether participants would participate
in the same experience again

| Answer Choices | Responses | Number of Participants |
|---|---|---|
| Absolutely | 57.89% | 11 |
| Probably | 15.79% | 3 |
| Not sure | 21.05% | 4 |
| Probably not | 0.00% | 0 |
| Never | 5.26% | 1 |
| | | Total: 19 |

FIG. 4 shows the survey results on the question “how soon would the participate like to participate in the same experience again”. Table 4 below further summarizes the data shown in FIG. 4.

TABLE 4
Answers to how soon the participants would
participate in the same experience again

| Answer Choices | Responses | Number of Participants |
|---|---|---|
| Tomorrow | 21.05% | 4 |
| In a few days | 5.26% | 1 |
| Next week | 0.00% | 0 |
| Next month | 47.37% | 9 |
| Next year | 21.05% | 4 |
| Never | 5.26% | 1 |
| | | Total: 19 |

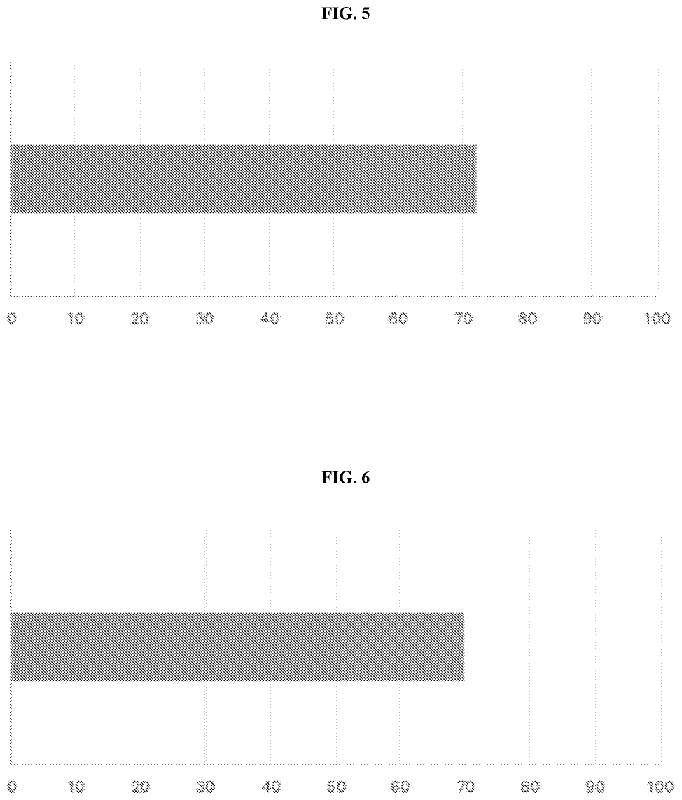

FIG. 5 shows the rating of the overall level of satisfaction (on a scale of 0-100) of the participants from this experience.

FIG. 6 shows survey results on how likely (on a scale of 0-100) the participants would recommend this experience to a friend or loved one.

### Conclusion

Overall, the participants had positive experiences from participating in the supported meditation experience as described herein.

### Example 3: Controlled Medication of Improved Combinations

A survey is conducted on 50 adults (25 male and 25 female) of age 21 or older on supported medication for 5 months.

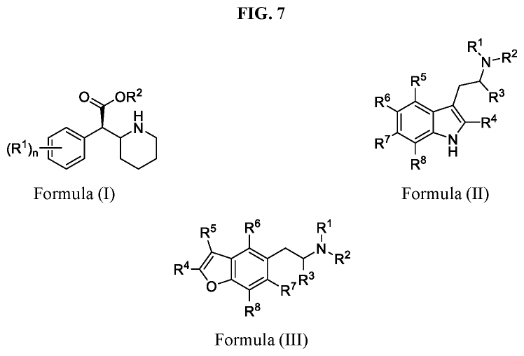

Over the 5 month period, individuals are administered either: (a) a combination of 10 mg of 4-acetoxy-N-methyl-N-ethyltryptamine (metacetin or 4-AcO-MET), 70 mg of 1-(benzofuran-5-yl)-N-methylpropan-2-amine (5-MAPB), and 10 mg of 2-fluoromethamphetamine (4F-MPH), or a combination with different amounts of the various compounds while maintaining the ratio described (see FIG. 7); (b) any one of 10 mg of 4-acetoxy-N-methyl-N-ethyltryptamine (metacetin or 4-AcO-ET), 70 mg of 1-(benzofuran-5-yl)-N-methylpropan-2-amine (5-MAPB), and 10 mg of 2-fluoromethamphetamine(4F-MPH) alone or in combination with only a single other constituent; or (c) a placebo made of pharmaceutically acceptable excipients and/or carriers.

Prior to administration, the subjects answer the Pre-experience questionnaire comprising at least some (if not all) of the questions in Table 5.

Following the administration period, the subjects answer the Post-experience questionnaire comprising at least some (if not all) of the questions in Table 5.

TABLE 5

| Sexuality Questions | Always Often Sometimes Rarely Never |
|---|---|
| Pre-experience questionnaire | |
| 1) I am satisfied with my sex life | 1 2 3 4 5 |
| 2) I have sexual fantasies | 1 2 3 4 5 |
| 3) I have sexual dreams | 1 2 3 4 5 |
| 4) I like to talk about things that concern sexuality | 1 2 3 4 5 |
| 5) I like to tell jokes involving sex | 1 2 3 4 5 |
| 6) I feel uninhibited towards sexuality | 1 2 3 4 5 |
| 7) I like to keep sexuality hidden | 5 4 3 2 1 |
| 8) I speak about sexuality with my partner | 1 2 3 4 5 |
| 9) I live sexuality in rigid manner | 5 4 3 2 1 |
| 10) I would live better without sexuality | 5 4 3 2 1 |
| 11) My sex life is planned | 5 4 3 2 1 |
| 12) I avoid situations that arouse my sexuality | 5 4 3 2 1 |
| 13) Sexuality creates worry for me | 5 4 3 2 1 |
| 14) I like watching movies or scenes involving sex | 1 2 3 4 5 |
| 15) I like to talk during sex | 1 2 3 4 5 |
| 16) I reach orgasm during sex | 1 2 3 4 5 |
| 17) During sex I “let go” | 1 2 3 4 5 |
| 18) Sexuality scares me | 5 4 3 2 1 |
| 19) I like to have an active role in my sexuality | 1 2 3 4 5 |
| Medical history questions | |
| 20) I have previous experience with MDMA? | Yes, No |
| 21) I have previous experience with psylocibin? | Yes, No |
| 22) I have previous experience with ritalin or | Yes, No |
| methylphenidate | |
| 23) I have a heart condition | Yes, No |
| 24) I have hypertension (high blood pressure) | Yes, No |
| 25) I have seizure disorder | Yes, No |
| 26) I have personal history or family history of | Yes, No |
| schizophrenia, psychotic break or psychosis | |
| 27) I have a mood disorder and am actively on SSRIs | Yes, No |
| 28) I experience erectile dysfunction | Yes, No |
| 29) I experience sexual pain | Yes, No |
| 30) I experience problems with sexual arousal or desire | Yes, No |
| 31) I struggle with orgasm | Yes, No |
| Post-experience questionnaire | |

1) Are you male or female?
2) What is your age?
3) What is your current weight?
4) What is that date of your experience?
5) Tell us about the experience
a. How many others did you have the experience with?
b. Were intentions set before the experience began?
c. What was the set and setting?
d. Was it a special occasion?
e. How did you feel about the length of the experience?
f. How did you feel the next day(s)?
6) What positive feelings did you experience?
a. Mystical experience
b. Unity
c. Soulfulness
d. Human Connection
e. Empathy
f. Compassion
g. Tactile enhancement
h. Comfort
i. Belonging
j. Sexual arousal
k. Increased libido or sexual desire
l. Euphoria
m. Passion
n. Ecstasy
o. Meaningfulness
p. Significance
q. Awe-inspiring
r. Enjoyability
s. Lucidity
t. Insightfulness
u. Awareness
v. Authenticity
w. Afterglow the next day
x. No comedown the next day or two
y. Capacity to orgasm maintained
z. Tactile enhancement
7) What negative experiences did you experience?
a. Edginess
b. Nausea
c. Jaw-clenching or grinding teeth (bruxism)
d. Paranoia
e. Negative thoughts
f. Comedown the next day or two
g. Flat emotions the next day
h. Tingling/burning sensation
i. Numbness in extremities
j. Insomnia
k. Muscle cramping
l. Blurred vision
m. Chills
n. sweating
o. Irritability
p. Anxiety
q. Hallucinations
r. Drowsiness
s. Lack of coordination
t. Dizziness
u. Headache
v. Dissociation
w. Hyperthermia
x. Erectile dysfunction or inability to orgasm
8) Please describe the best moments of the experience
9) Please describe the worst moments of the experience
10) Have you felt a persistent shift in your perception or state since the experience?
If so, was it negative or positive?
11) Did the experience effect any of your personal relationships? (i.e fell in love,
got engaged, forgave someone, broke up, separated, etc . . . ) If so, please explain
12) Would you like to have this experience again?
13) How soon would you like to have this experience again?
14) Please rate your overall level of satisfaction with this experience
(0 being the worst and 100 being the best)
15) How likely would you be (on a scale of 0-100) to recommend this experience
to a friend or loved one?
16) Please sum up your experience using one to three sentences?

It is observed that subjects given the described combination experienced exaggerated positive feelings while experiencing little-to-no negative feelings, relative to both the placebo group and the groups wherein the subjects ingested one or two members of the described combination. It is observed that several subjects report experiencing one or more positive feelings that are commonly associated with ingesting MDMA (confirmed by subject-testimony) while also not experiencing few or none of the negative feelings commonly associated with ingesting MDMA (confirmed by subject-testimony).

### Example 4: Biochemic Analysis of a Combination of Compound 1, Compound 2, and Compound 3

Studies are conducted testing the biochemistry of the combinations disclosed herein. Excitatory (and, in some cases, inhibitory) assays are run on serotonin receptors: 5ht2a, 5ht2c, and 5ht1a; dopamine receptors: D1, D2, D3; adrenergic receptors: α1, α2; serotonin transporter (SERT); norepinephrine transporter (NET); dopamine transporter (DAT); histamine receptor: H1, Monoamine transporters: VMAT1, VMAT2.

In the studies, (a) a combination of 10 mg of 4-acetoxy-N-methyl-N-ethyltryptamine (metacetin or 4-AcO-MET), 70 mg of 1-(benzofuran-5-yl)-N-methylpropan-2-amine (5-MAPB), and 10 mg of 2-fluoromethamphetamine (4F-MPH), or a combination with different amounts of the various compounds while maintaining the ratio described; (b) any one of 10 mg of 4-acetoxy-N-methyl-N-ethyltryptamine (metacetin or 4-AcO-MET), 70 mg of 1-(benzofuran-5-yl)-N-methylpropan-2-amine (5-MAPB), and 10 mg of 2-fluoromethamphetamine (4F-MPH) alone or in combination with only a single other constituent; or (c) a placebo made of pharmaceutically acceptable excipients and/or carriers are used on any one of the aforementioned receptors alone, or in any combination.

The results of these experiments show that administration of any single Compound of the disclosed combinations, or administration of a combination consisting of two (of the three) Compounds result in insignificant or no change in the activity of at least a subset of the receptors tested. However, administration with the disclosed combination resulted in augmented activity in at least a subset of receptors tested. This subset is known to correlate with positive responses to known sexual disorders, including hypoactive sexual desire disorder, sexual arousal disorder, sexual aversion, anorgasmia, vaginismus, dyspareunia, erectile dysfunction, diminished libido, and abnormal ejaculation.

### Example 5: Neurotoxic Analysis of a Combination of Compound 1, Compound 2, and Compound 3

It is indicated that the experiences felt by users of the combinations disclosed herein resemble the positive feelings experienced when ingested MDMA. Studies are conducted to test the neurotoxic outcomes of administration of the combinations disclosed herein to mammalian subjects.

In these studies, (a) a combination of 10 mg of 4-acetoxy-N-methyl-N-ethyltryptamine (metacetin or 4-AcO-MET), 70 mg of 1-(benzofuran-5-yl)-N-methylpropan-2-amine (5-MAPB), and 10 mg of 2-fluoromethamphetamine (4F-MPH), or a combination with different amounts of the various compounds while maintaining the ratio described; (b) any one of 10 mg of 4-acetoxy-N-methyl-N-ethyltryptamine (metacetin or 4-AcO-MET), 70 mg of 1-(benzofuran-5-yl)-N-methylpropan-2-amine (5-MAPB), and 10 mg of 2-fluoromethamphetamine (4F-MPH) alone or in combination with only a single other constituent; (c) MDMA; or (d) a placebo made of pharmaceutically acceptable excipients and/or carriers are used as experimental groups in known neurochemical and/or neurotoxic assays used to study the same derived from MDMA administration. An example assay is found in Górska et al. (Neurotoxicity Research volume 33, pages 532-548 (2018)) which is incorporated by reference herein in its entirety.

It is observed that any one of 10 mg of 4-acetoxy-N-methyl-N-ethyltryptamine (metacetin or 4-AcO-MET), 70 mg of 1-(benzofuran-5-yl)-N-methylpropan-2-amine (5-MAPB), and 10 mg of 2-fluoromethamphetamine (4F-MPH) alone or in combination (including the combination of all 3 Compounds)

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A combination comprising:
a. one or more compounds of Formula (I) 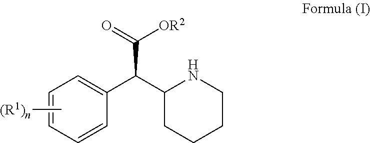
or pharmaceutically acceptable salts, solvates, or hydrates thereof, wherein
each R¹ is independently selected from hydrogen, halo, C₁₋₆alkyl, C₁₋₆haloalkyl, C₁₋₆ alkoxy, C₁₋₆haloalkoxy, —OR³, —C(O)R³, and —C(O)OR³;
R² is selected from hydrogen and C₁₋₆alkyl;
R³ is selected from hydrogen, C₁₋₆alkyl, C₃₋₆cycloalkyl, and C₆₋₁₀aryl; and
n is selected from 1, 2, 3, 4, and 5; and
b. one or more compounds of Formula (II) 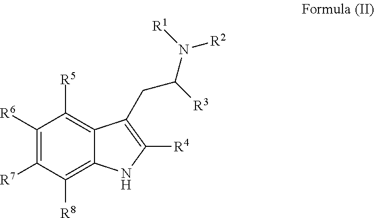
or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆-haloalkoxy, and —N(R⁹)₂;
R⁴, R⁵, R⁶, R⁷, and R⁸ are independently selected from hydrogen, halo, C₁₋₆alkyl, C₁₋₆ haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

2. The combination of claim 1, wherein the combination comprises about 1 mg to about 20 mg of said one or more compounds of Formula (I) or pharmaceutically acceptable salts, solvates, or hydrates thereof.

3. The combination of any one of the preceding claims, wherein the combination comprises about 1 mg to about 25 mg of said one or more compounds of Formula (II) or pharmaceutically acceptable salts, solvates, or hydrates thereof.

4. A combination comprising:
a. one or more compounds of Formula (IV): 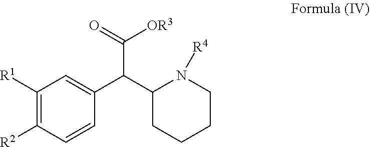
or a pharmaceutically acceptable salts, solvates, or hydrates thereof, wherein:
R¹ and R² are independently selected from hydrogen, halo, C₁₋₆alkyl, C₁₋₆haloalkyl, C₁₋₆alkoxy, C₁₋₆haloalkoxy, —OR⁵, —C(O)R⁵, and —C(O)OR⁵; or R¹ and R² together with the atoms to which they are attached join to form 5-8 membered cycloalkyl, 5-8 membered heterocycloalkyl, or 6-membered aryl;
R³ is selected from hydrogen and C1-6alkyl;
R⁴ is selected from hydrogen, C₁₋₆alkyl, and —C(O)OR⁵; and
R⁵ is selected from hydrogen and C₁₋₆alkyl, wherein C₁₋₆alkyl is optionally substituted with aryl, substituted aryl, heteroaryl, and substituted heteroaryl; and
b. one or more compounds of Formula (V) 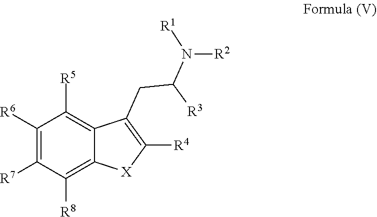
or pharmaceutically acceptable salts, solvates, or hydrates thereof, wherein
X is O or NH;
R¹ and R² are independently selected from C₁₋₆alkyl and C₂₋₆alkenyl, wherein the C₁₋₆ alkyl and C₂₋₆alkenyl are optionally substituted with C₆₋₁₀aryl C₃₋₆cycloalkyl, or C₃₋₆heterocycloalkyl; or R¹ and R² together with the nitrogen to which they are attached join to form a 3-6 membered heterocycloalkyl;
R³ is selected from hydrogen, halo, C₁₋₆alkyl, C₁₋₆haloalkyl, C₁₋₆alkoxy, C₁₋₆-haloalkoxy, and —N(R⁹)₂;
R⁴, R⁵, R⁶, R⁷, and R⁸ are independently selected from hydrogen, halo, C₁₋₆alkyl, C₁₋₆ haloalkyl, C₁₋₆alkoxy, C₁₋₆haloalkoxy, —N(R⁹)₂, —OR⁹, —OC(O)R⁹, —C(O)R⁹, —C(O)OR⁹ and —OP(O)(OH)₂; and
R⁹ is selected from hydrogen, C₁₋₆alkyl, C₁₋₆haloalkyl C₃₋₆cycloalkyl, and C₆₋₁₀aryl.

5. The combination of claim 4, wherein the combination comprises about 1 mg to about 20 mg of said one or more compounds of Formula (IV) or pharmaceutically acceptable salts, solvates, or hydrates thereof.

6. The combination of claim 4, wherein the combination comprises about 1 mg to about 25 mg of said one or more compounds of Formula (V) or pharmaceutically acceptable salts, solvates, or hydrates thereof.

7. The combination of any one of the preceding claims, wherein the combination further comprises one or more compounds of Formula (III) 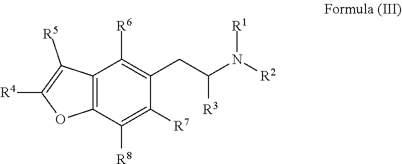
or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂;
R⁴, R⁵, R⁶, R⁷, and R⁸ are independently selected from hydrogen, halo, C₁₋₆alkyl, C₁₋₆-haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

8. The combination of any one of the preceding claims, wherein the combination comprises about 1 mg to about 75 mg of said one or more compounds of Formula (III) or pharmaceutically acceptable salts, solvates, or hydrates thereof.

9. The combination of any one of the preceding claims, wherein the combination further comprises one or more compounds of Formula (VI) 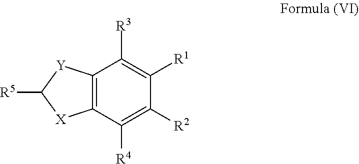
or pharmaceutically acceptable salts, solvates, or hydrates thereof, wherein
X and Y are independently selected from O and CH₂;
R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶;
R³ and R⁴ are independently selected from hydrogen, halogen, C₁₋₆alkyl, C₁₋₆haloalkyl, C₁₋₆ alkoxy, C₁₋₆haloalkoxy, —N(R⁶)₂, —OR⁶, —OC(O)R⁶, —C(O)R⁶, and —C(O)OR⁶;
R⁵ is selected from hydrogen, halogen, —N(R⁶)₂, and —OR⁶; and
R⁶ is selected from hydrogen, C₁₋₆alkyl, C₁₋₆haloalkyl C₃₋₆cycloalkyl, and C₆₋₁₀aryl.

10. The combination of any one of the preceding claims, wherein the combination further comprises one or more compounds of Formula (VII) 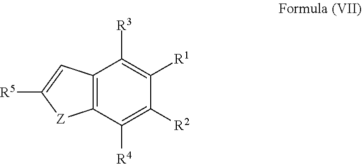
or pharmaceutically acceptable salts, solvates, or hydrates thereof, wherein
Z is selected from NH or O;
R¹ and R² are independently selected from hydrogen, halogen, C₁₋₆alkyl and C₂₋₆alkenyl, wherein the C₁₋₆alkyl and C₂₋₆alkenyl are optionally substituted with —N(R⁶)₂, —N(R⁶)OH, —OR⁶, —OC(O)R⁶, —C(O)R⁶, and —C(O)OR⁶;
R³ and R⁴ are independently selected from hydrogen, halogen, C₁₋₆alkyl, C₁₋₆haloalkyl, C₁₋₆ alkoxy, C₁₋₆haloalkoxy, —N(R⁶)₂, —OR⁶, —OC(O)R⁶, —C(O)R⁶, and —C(O)OR⁶;
R⁵ is selected from hydrogen, halogen, —N(R⁶)₂, and —OR⁶; and
R⁶ is selected from hydrogen, C₁₋₆alkyl, C₁₋₆haloalkyl C₃₋₆cycloalkyl, and C₆₋₁₀aryl.

11. The combination of any of the preceding claims, wherein the combination further comprises one or more compounds selected

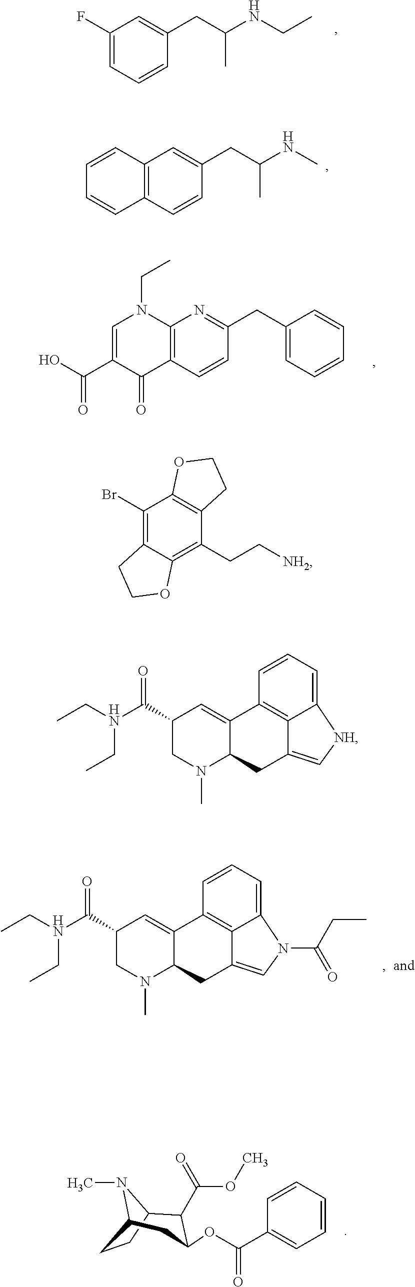

12. The combination of any one of the preceding claims, wherein the combination comprises about 1 mg to about 75 mg of said one or more compounds of Formula (VI) or pharmaceutically acceptable salts, solvates, or hydrates thereof.

13. The combination of any one of the preceding claims, wherein the combination comprises about 1 mg to about 75 mg of said one or more compounds of Formula (VII) or pharmaceutically acceptable salts solvates, hydrates thereof.

14. The combination of any one of the preceding claims, wherein said one or more compounds of Formula (I) comprises one or more compounds of Formula (I-A) 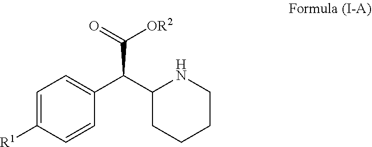
or pharmaceutically acceptable salts, solvates, or hydrates thereof, wherein
each R¹ is independently selected from hydrogen, halo, C₁₋₆alkyl, C₁₋₆haloalkyl, C₁₋₆alkoxy, C₁₋₆haloalkoxy, —OR³, —C(O)R³, and —C(O)OR³;
R² is selected from hydrogen and C₁₋₆alkyl;
R³ is selected from hydrogen, C₁₋₆alkyl, C₃₋₆cycloalkyl, and C₆₋₁₀aryl; and
n is selected from 1, 2, 3, 4, and 5.

15. The combination of any one of the preceding claims, wherein said one or more compounds of Formula (II) comprises one or more compounds of Formula (II-A) 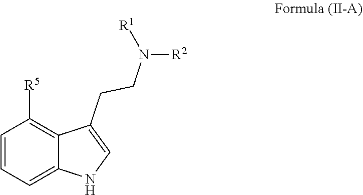
or pharmaceutically acceptable salts, solvates, or hydrates thereof, wherein
R¹ and R² are independently selected from C₁₋₆alkyl and C₂₋₆alkenyl;
R⁵ is selected from hydrogen, halo, C₁₋₆alkyl, C₁₋₆haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

16. The combination of any one of the preceding claims, wherein said one or more compounds of Formula (III) comprises one or more compounds of Formula (III-A) 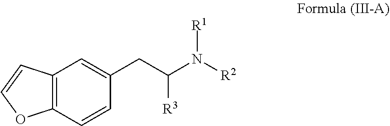
or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂; and
R⁹ is selected from hydrogen, C₁₋₆alkyl, C₁₋₆haloalkyl C₃₋₆cycloalkyl, and C₆₋₁₀aryl.

17. The combination of any one of the preceding claims, wherein said one or more compounds of Formula (I) comprise Compound 1 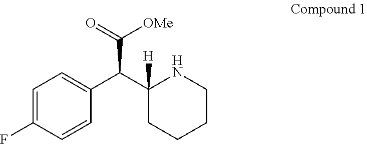 [SMILES: COC(=O)[C@H](c1ccc(F)cc1)[C@H]1CCCCN1]
or a pharmaceutically acceptable salt, solvate, or hydrate thereof.

18. The combination of any one of the preceding claims, wherein said one or more compounds of Formula (II) comprise Compound 2 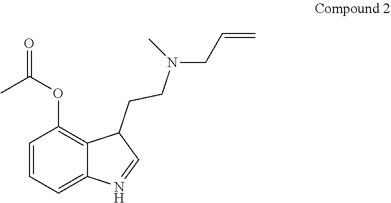 [SMILES: C=CCN(C)CCC1C=Nc2cccc(OC(C)=O)c21]
or a pharmaceutically acceptable salt, solvate, or hydrate thereof.

19. The combination of any one of the preceding claims, wherein said one or more compounds of Formula (III) comprise Compound 3 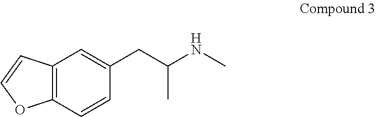
or a pharmaceutically acceptable salt, solvate, or hydrate thereof.

20. The combination of any one of the preceding claims, wherein the compound of Formula I is selected from: 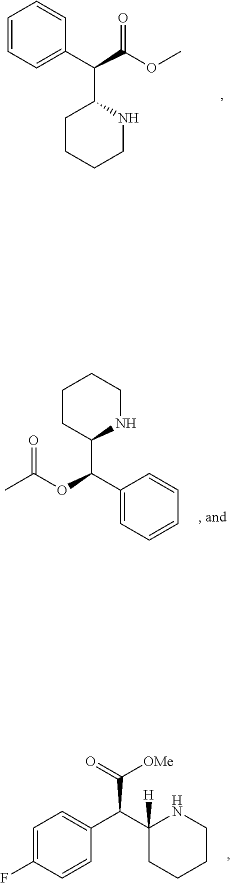
or pharmaceutically acceptable salts, solvates, or hydrates thereof.

21. The combination of any one of the preceding claims, wherein the compound of Formula II is selected from: 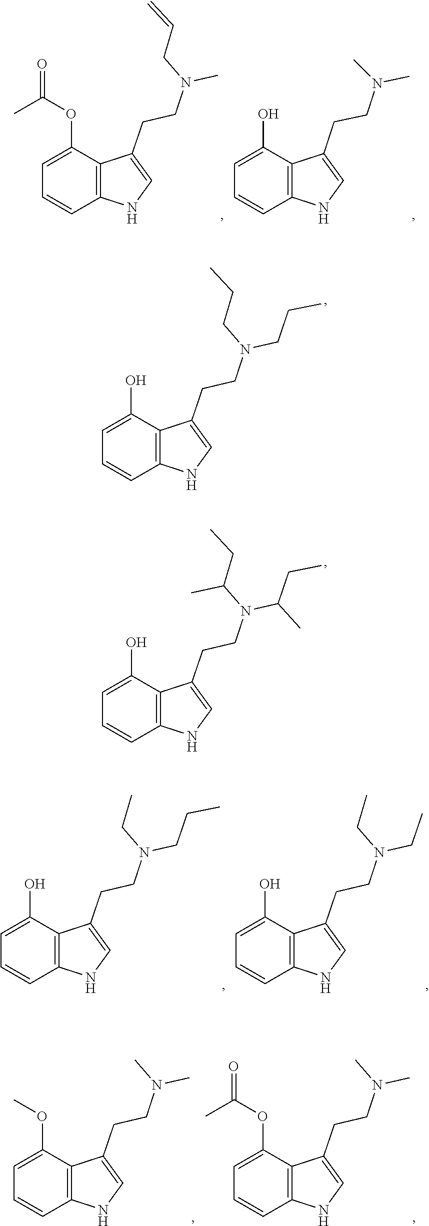 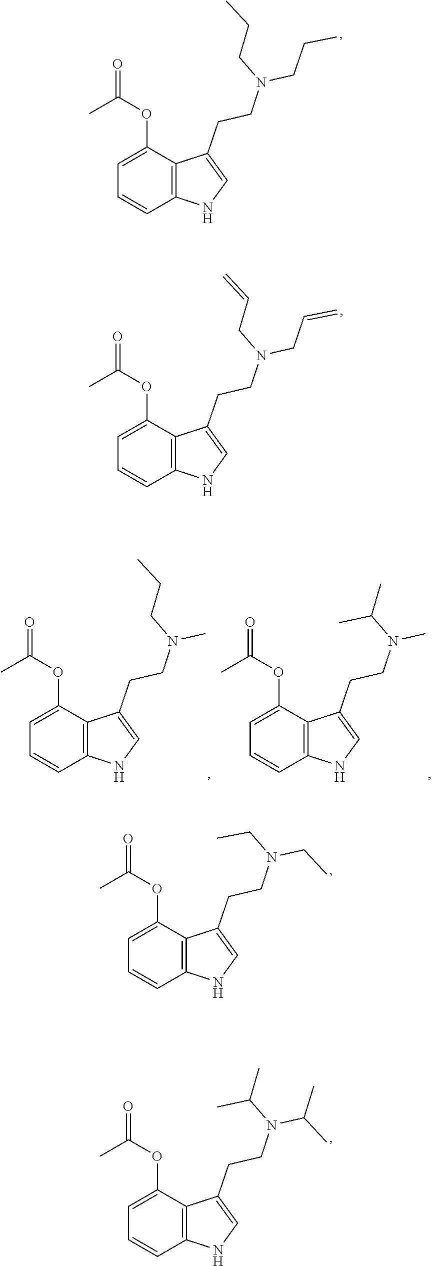 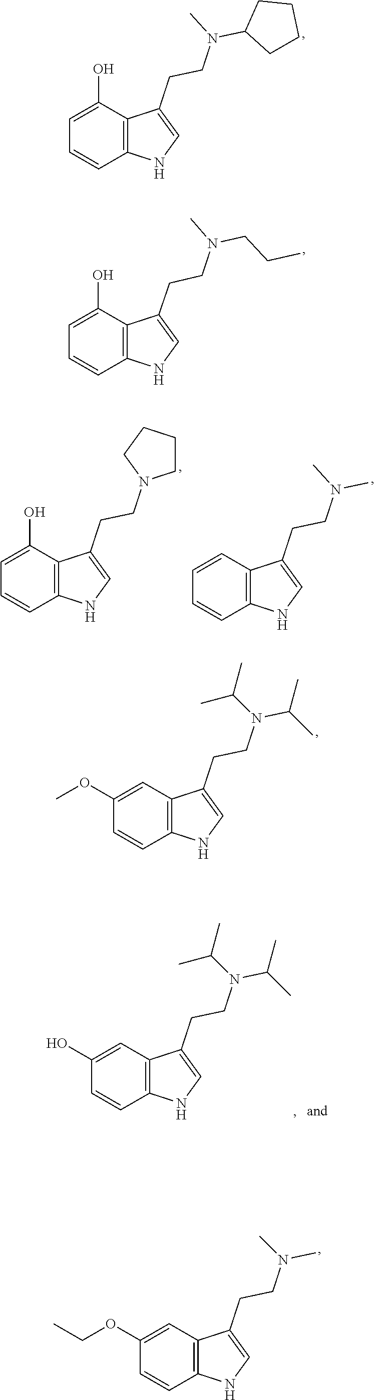
or pharmaceutically acceptable salts, solvates, or hydrates thereof.

22. The combination of any one of the preceding claims, wherein the compound of Formula (III) is selected from: 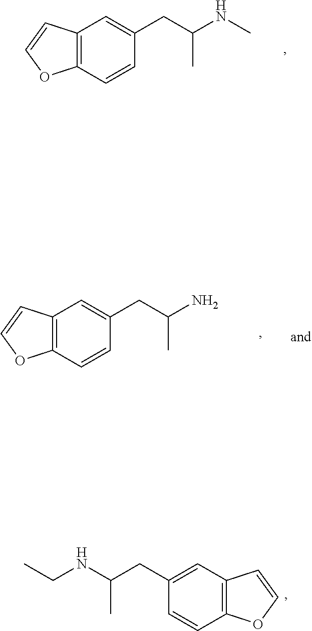
or pharmaceutically acceptable salts, solvates, or hydrates thereof.

23. The combination of any one of the preceding claims, wherein the compound of Formula (IV) is selected from:

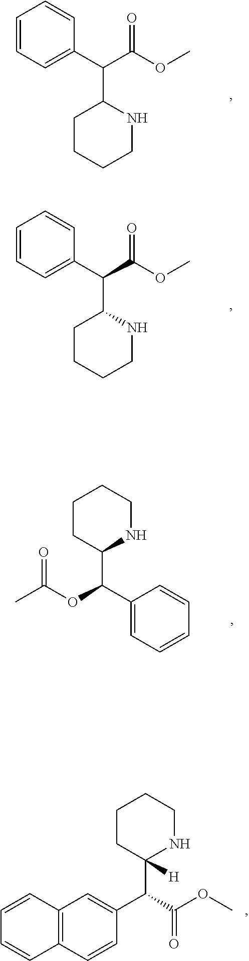

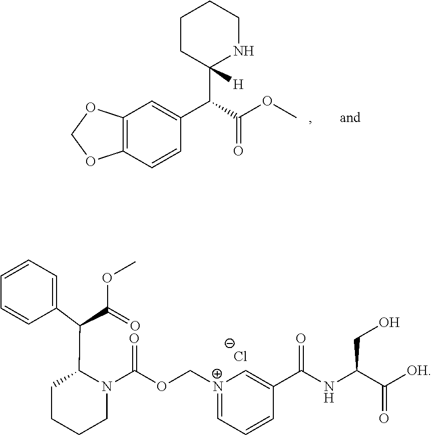 [SMILES: COC(=O)[C@H](c1ccc2c(c1)OCO2)[C@@H]1CCCCN1 ; COC(=O)[C@H](c1ccccc1)[C@H]1CCCCN1C(=O)OC[n+]1cccc(C(=O)N[C@@H](CO)C(=O)O)c1 ; [Cl-]]

24. The combination of any one of the preceding claims, wherein the compound of Formula (V) is selected from: 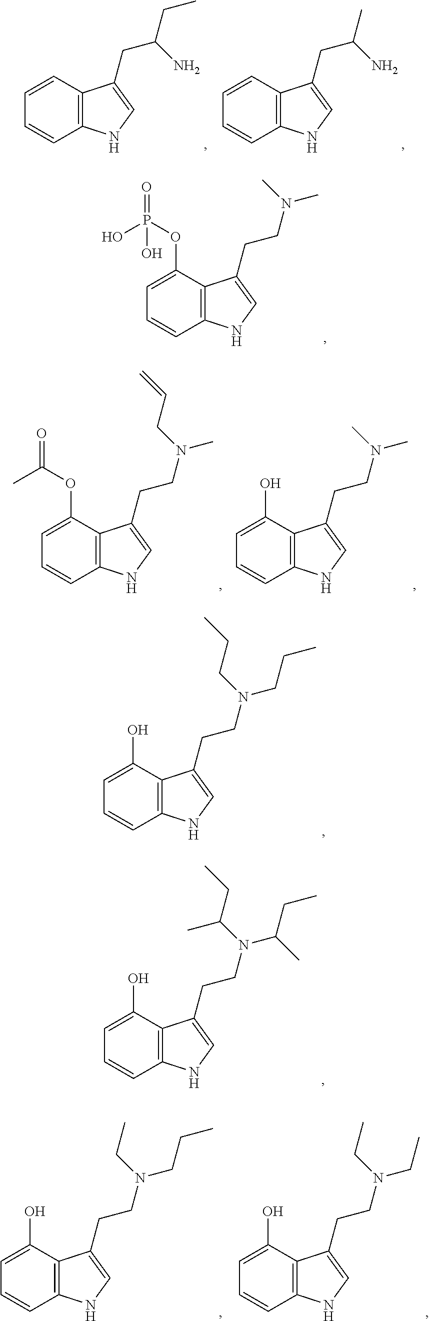 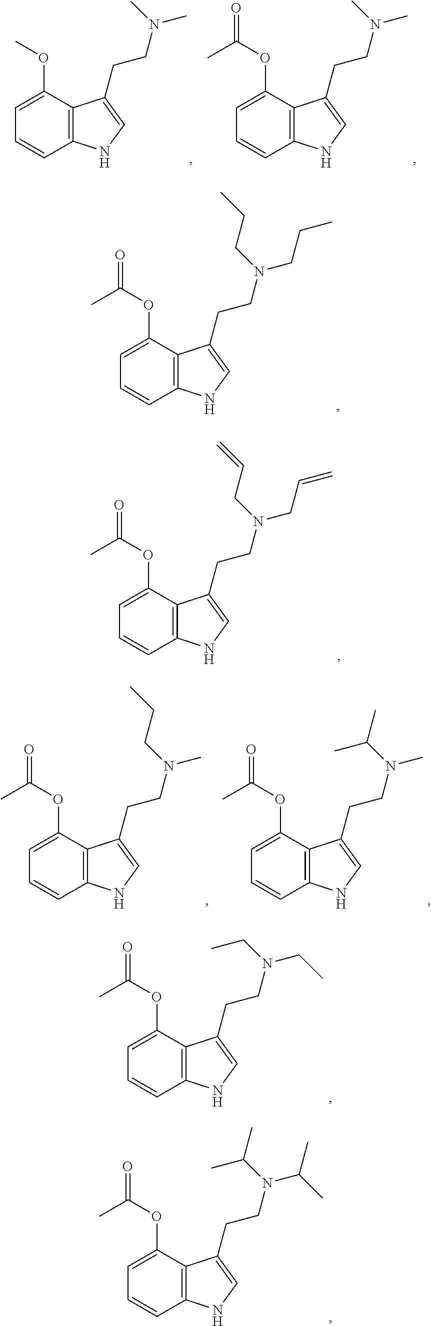 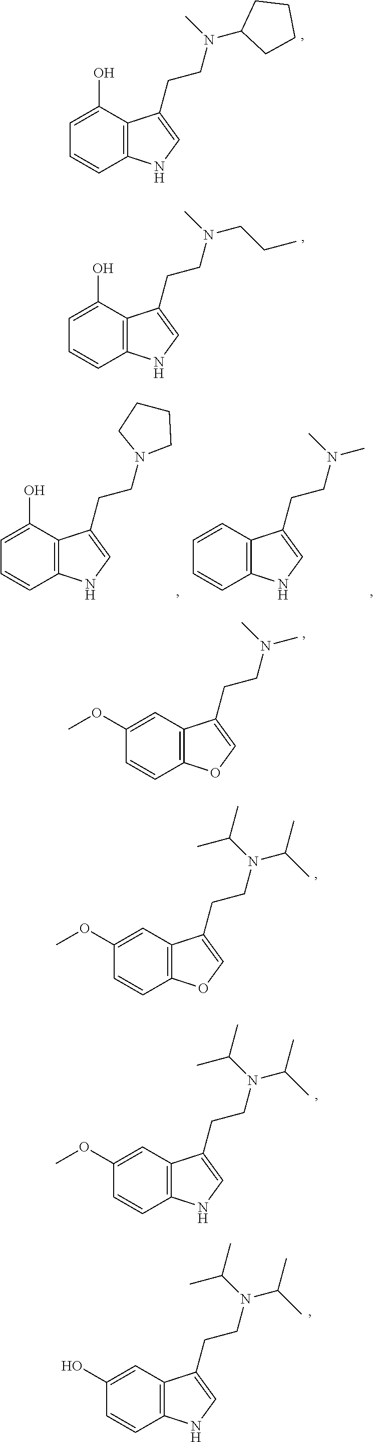 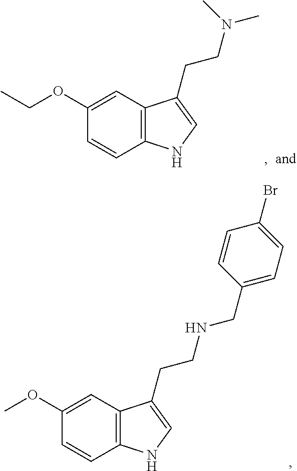
or pharmaceutically acceptable salts, solvates, or hydrates thereof.

25. The combination of any one of the preceding claims, wherein the compound of Formula VI is selected from: 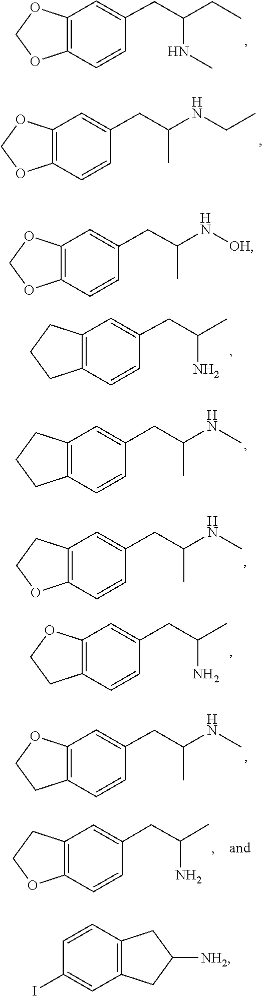
or pharmaceutically acceptable salts, solvates, or hydrates thereof.

26. The combination of any one of the preceding claims, wherein the compound of Formula VII is selected from:

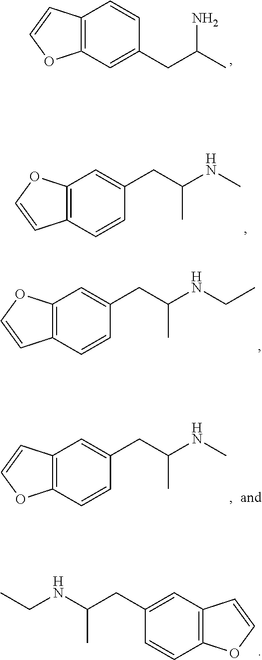

27. A combination comprising one or more compounds selected from: 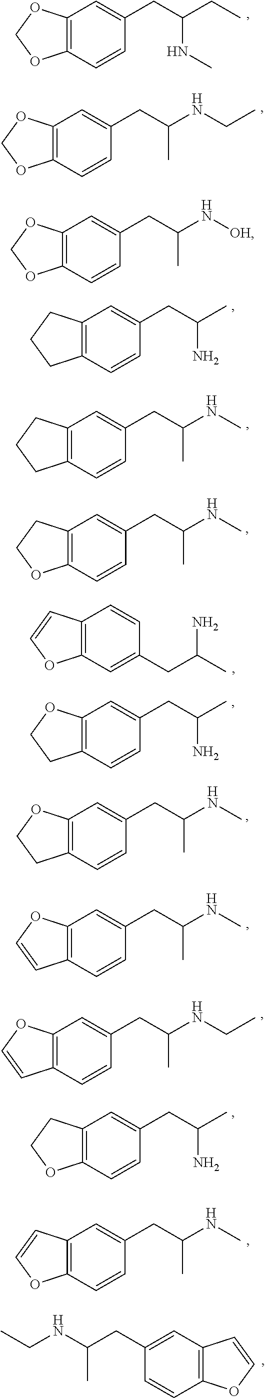 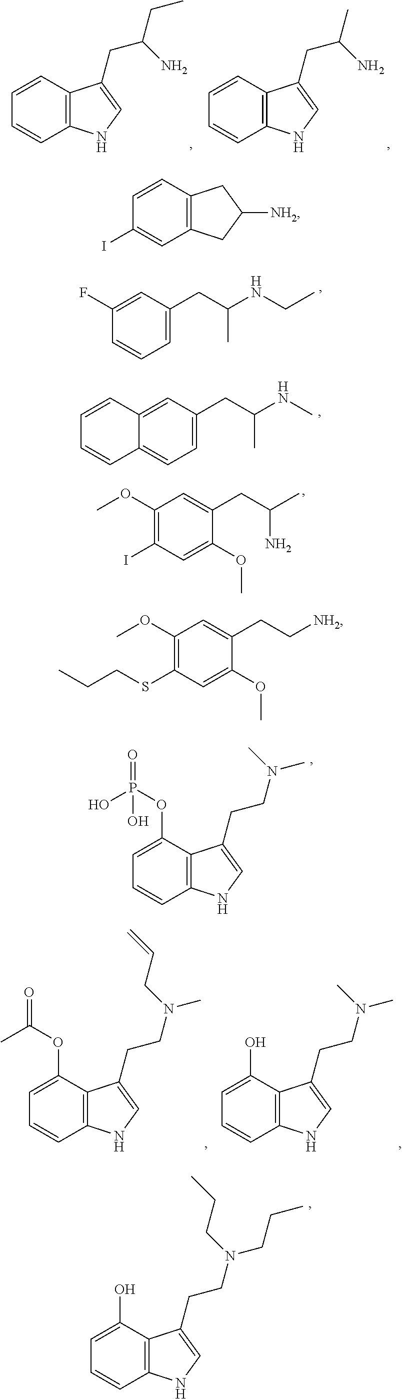 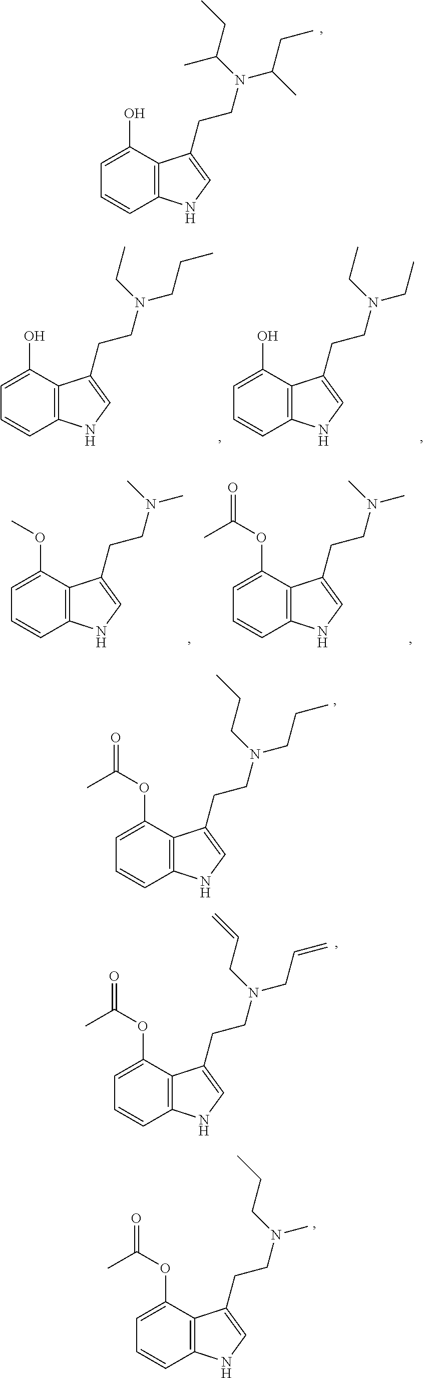 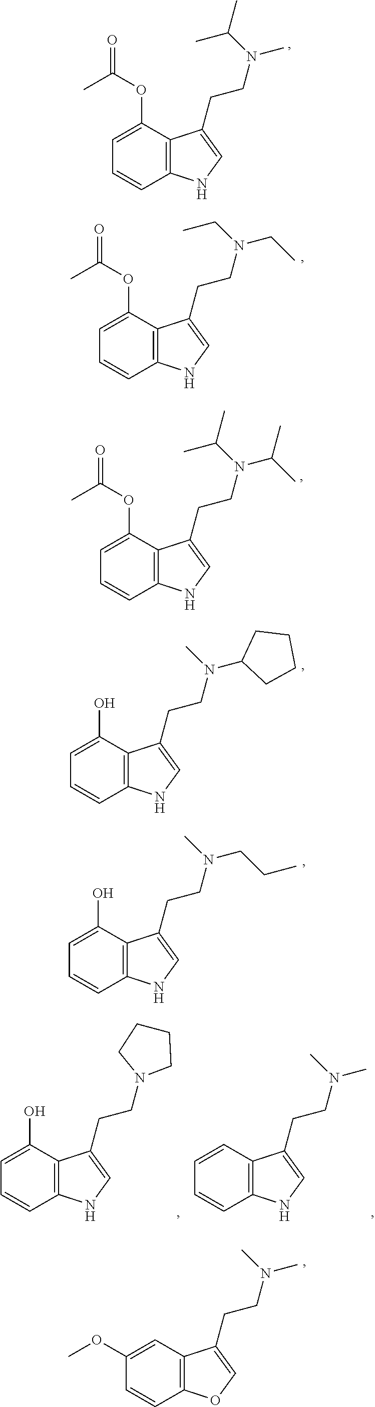 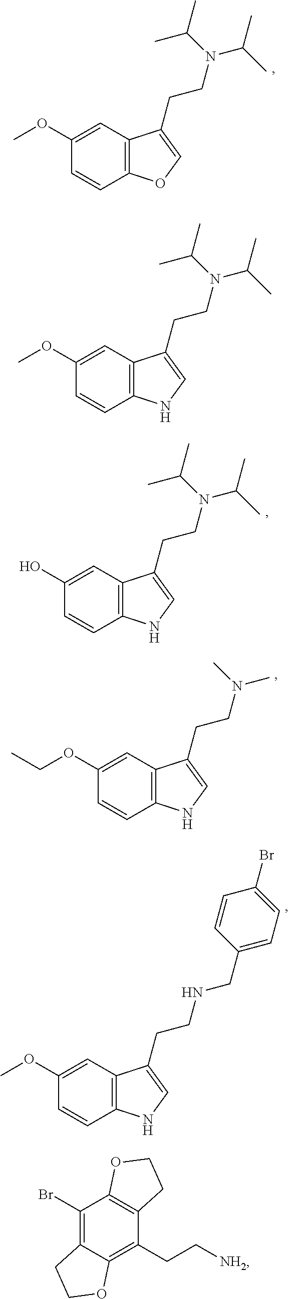 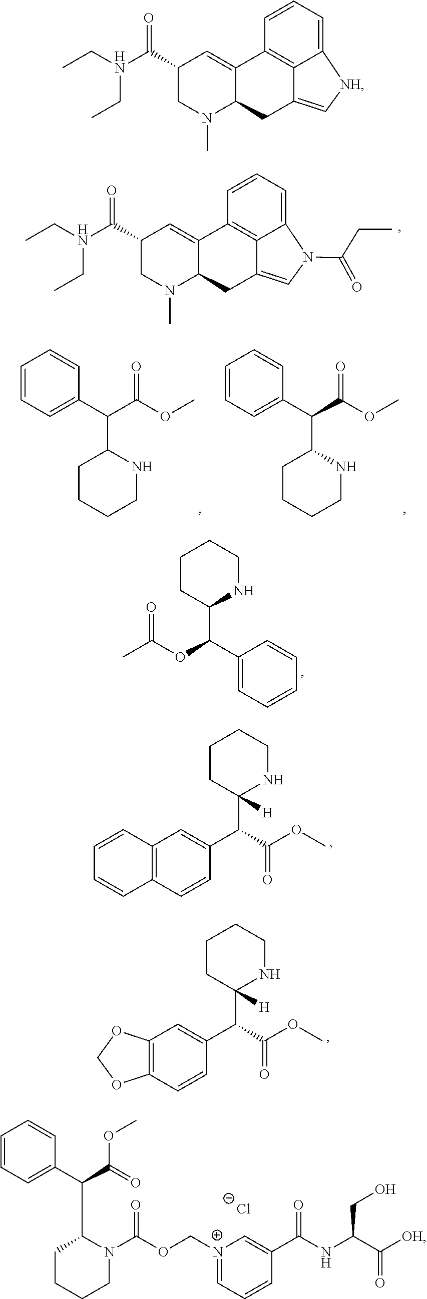 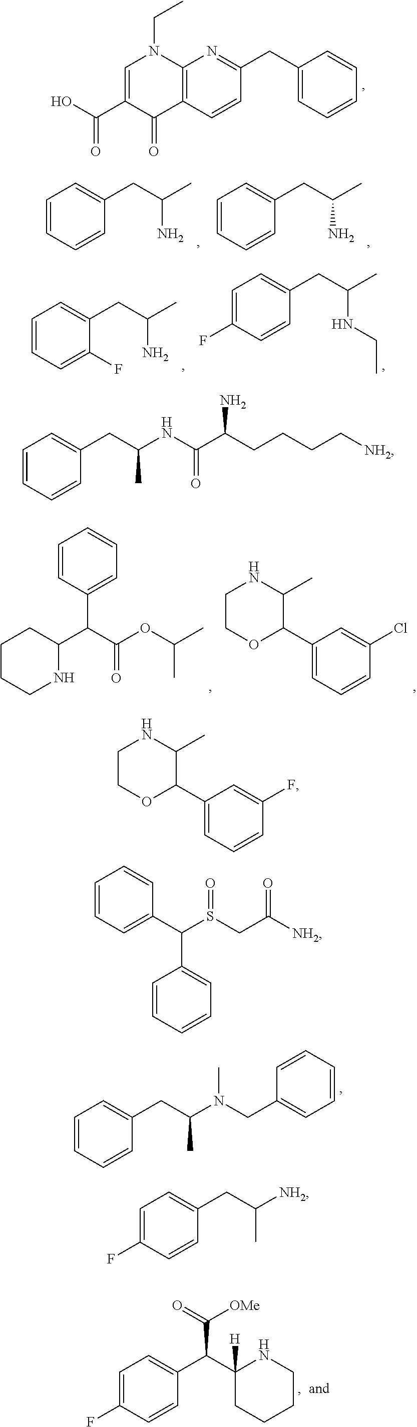 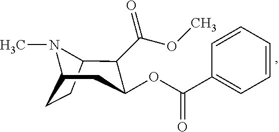
or pharmaceutically acceptable salts, solvates, or hydrates thereof.

28. A pharmaceutical composition, comprising (A) the combination of any one of claims 1-27, or pharmaceutically acceptable salts, solvates, or hydrates thereof, and (B) a pharmaceutically acceptable carrier.

29. An oral unit dosage form comprising (A) the combination of any one of claims 1-27 or the pharmaceutical composition of claim 28, and (B) a pharmaceutically acceptable carrier.

30. The oral unit dosage form of claim 29, wherein the oral unit dosage form is a tablet.

31. The oral unit dosage form of claim 29, wherein the oral unit dosage form is a liquid.

32. A method for treating a sexual disorder in a subject in need thereof, the method comprising administering to said subject:
the combination of any one of claims 1-27, or pharmaceutically acceptable salts, solvates, or hydrates thereof;
the pharmaceutical composition of claim 28; or
the oral unit dosage form of any one of claims 29-31.

33. The method of claim 32, wherein said sexual disorder is selected from hypoactive sexual desire disorder, sexual arousal disorder, sexual aversion, anorgasmia, vaginismus, dyspareunia, erectile dysfunction, diminished libido, and abnormal ejaculation.

34. A method for treating a disease, a disorder, or a condition in a subject in need thereof, the method comprising administering to said subject:
the combination of any one of claims 1-27, or pharmaceutically acceptable salts, solvates, or hydrates thereof;
the pharmaceutical composition of claim 28; or
the oral unit dosage form of any one of claims 29-31.

35. The method of claim 34, wherein the disease, the disorder, or the condition is selected from sexual abuse, mood disorders, phobia disorders, dissociative disorders, stress disorders, personality disorders, obsessive compulsive disorders, Tourette's disorder, grief, autism, eating disorders, alcohol use disorder, conduct disorder, conversion disorder, and addiction.

36. The method of claim 35, wherein the sexual abuse is selected from adult sexual disorder and child sexual disorder.

37. The method of claim 35, wherein the mood disorder is selected from persistent depressive disorder or dysthymia, major depressive disorder, cyclothymic disorder, premenstrual dysphoric disorder, generalized anxiety disorder, and panic disorder.

38. The method of claim 35, wherein the phobia disorder is selected from phobia of animal, phobia of blood-injection-injury, phobia of blood, phobia of injections or transfusions, phobia of injury, phobia of medical care, phobia of natural environment, and situational phobias.

39. The method of claim 35, wherein the stress disorder is selected from acute stress disorder, posttraumatic stress disorder, unspecified trauma- and stressor-related disorder, nightmare disorder, adjustment disorder, reactive attachment disorder, and disinhibited attached disorder.

40. The method of claim 35, wherein the personality disorder is selected from antisocial behavior, antisocial personality disorder, avoidant personality disorder, borderline personality disorder, narcissistic personality disorder, and obsessive-compulsive personality disorder.

41. The method of claim 35, wherein the eating disorder is selected from avoidant/restrictive food intake disorder, binge-eating disorder, body dysmorphic disorder, bulimia nervosa, and anorexia nervosa.

42. The method of claim 35, wherein the addiction is selected from tobacco use addiction, opioid use disorder, and alcohol use disorder.

43. A combination comprising two or more compounds useful for the treatment of one or more sexual disorders, or pharmaceutically acceptable salts, solvates, or hydrates thereof, wherein said two or more compounds comprise an optionally fused benzene ring.

44. The combination of claim 43, comprising three or more compounds useful for the treatment of one or more sexual disorders, wherein said three or more compounds comprise an optionally fused benzene ring.

45. The combination of claim 43 or 44, wherein said compounds are independently selected from phenethylamines, tryptamines, benzofurans, and amphetamines.

46. The combination of any one of claims 43-45, wherein at least one of said two or more compounds comprises Compound 1 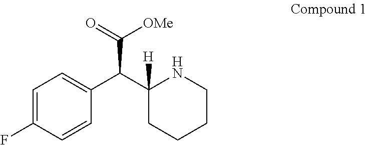 [SMILES: COC(=O)[C@H](c1ccc(F)cc1)[C@H]1CCCCN1]
or a pharmaceutically acceptable salt, solvate, or hydrate thereof.

47. The combination of any one of claims 43-45, wherein at least one of said two or more compounds comprises Compound 2 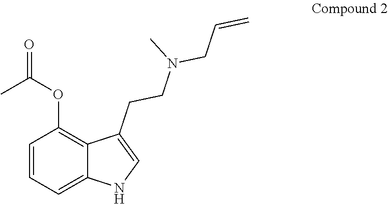 [SMILES: C=CCN(C)CCc1c[nH]c2cccc(OC(C)=O)c12]
or a pharmaceutically acceptable salt, solvate, or hydrate thereof.

48. The combination of any one of claims 43-45, at least one of said two or more compounds comprises Compound 3 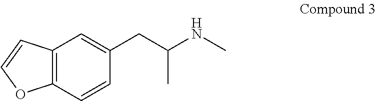
or a pharmaceutically acceptable salt, solvate, or hydrate thereof.

49. The combination of claim 46, wherein Compound 1 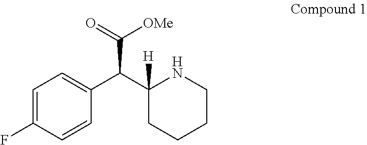 [SMILES: COC(=O)[C@H](c1ccc(F)cc1)[C@H]1CCCCN1]
or a pharmaceutically acceptable salt, solvate, or hydrate thereof, is present in the combination at about 1 mg to about 20 mg.

50. The combination of claim 47, wherein Compound 2 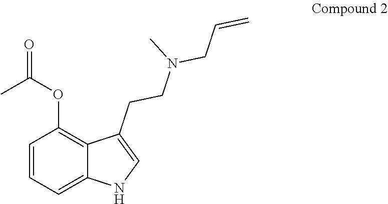 [SMILES: C=CCN(C)CCc1c[nH]c2cccc(OC(C)=O)c12]
or a pharmaceutically acceptable salt, solvate, or hydrate thereof, is present in the combination at about 1 mg to about 25 mg.

51. The combination of claim 48, wherein Compound 3 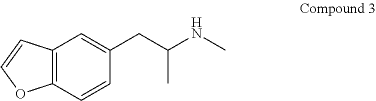
or a pharmaceutically acceptable salt, solvate, or hydrate thereof, is present in the combination at about 1 mg to about 75 mg.

52. A pharmaceutical composition, comprising (A) the combination of any one of claims 43-51, or pharmaceutically acceptable salts, solvates, or hydrates thereof, and (B) a pharmaceutically acceptable carrier.

53. An oral unit dosage form comprising (A) the combination of any one of claims 43-51, or the pharmaceutical composition of claim 52, and (B) a pharmaceutically acceptable carrier.

54. The oral unit dosage form of claim 53, wherein the oral unit dosage form is a tablet.

55. The oral unit dosage form of claim 53, wherein the oral unit dosage form is a liquid.

56. A combination comprising at least two compounds independently selected from empathogens, stimulants, and tryptamines.

57. The combination of claim 56, wherein the combination comprises an empathogen, a stimulant, and a tryptamine.

58. A method for treating a sexual disorder in a subject in need thereof, the method comprising administering to said subject:
the combination of any one of claims 43-51, or pharmaceutically acceptable salts, solvates, or hydrates thereof,
the pharmaceutical composition of claim 52;
the oral unit dosage form of any one of claims 53-55; or
the combination of claim 56 or 57.

59. The method of claim 58, wherein said sexual disorder is selected from hypoactive sexual desire disorder, sexual arousal disorder, sexual aversion, anorgasmia, vaginismus, dyspareunia, erectile dysfunction, diminished libido, and abnormal ejaculation.

60. A method for enhancing sexual drive in a subject, the method comprising administering to said subject:
the combination of any one of claims 43-51, or pharmaceutically acceptable salts, solvates, or hydrates thereof;
the pharmaceutical composition of claim 52;
the oral unit dosage form of any one of claims 53-55; or
the combination of claim 56 or 57.

61. A method for treating a disease, a disorder, or a condition in a subject in need thereof, the method comprising administering to said subject:
the combination of any one of claims 43-51, or pharmaceutically acceptable salts, solvates, or hydrates thereof;
the pharmaceutical composition of claim 52; or
the oral unit dosage form of any one of claims 53-55.

62. The method of claim 61, wherein the disease, the disorder, or the condition is selected from sexual abuse, mood disorders, phobia disorders, dissociative disorders, stress disorders, personality disorders, obsessive compulsive disorders, tourette's disorder, grief, autism, eating disorders, alcohol use disorder, conduct disorder, conversion disorder, and addiction.

63. The method of claim 61, wherein the sexual abuse is selected from adult sexual disorder and child sexual disorder.

64. The method of claim 61, wherein the mood disorder is selected from persistent depressive disorder or dysthymia, major depressive disorder, cyclothymic disorder, premenstrual dysphoric disorder, generalized anxiety disorder, and panic disorder.

65. The method of claim 61, wherein the phobia disorder is selected from phobia of animal, phobia of blood-injection-injury, phobia of blood, phobia of injections or transfusions, phobia of injury, phobia of medical care, phobia of natural environment, and situational phobias.

66. The method of claim 61, wherein the stress disorder is selected from acute stress disorder, posttraumatic stress disorder, unspecified trauma- and stressor-related disorder, nightmare disorder, adjustment disorder, reactive attachment disorder, and disinhibited attached disorder.

67. The method of claim 61, wherein the personality disorder is selected from antisocial behavior, antisocial personality disorder, avoidant personality disorder, borderline personality disorder, narcissistic personality disorder, and obsessive-compulsive personality disorder.

68. The method of claim 61, wherein the eating disorder is selected from avoidant/restrictive food intake disorder, binge-eating disorder, body dysmorphic disorder, bulimia nervosa, and anorexia nervosa.

69. The method of claim 61, wherein the addiction is selected from tobacco use addiction, opioid use disorder, and alcohol use disorder.

70. A combination comprising:
a. one or more compounds of Formula (I) 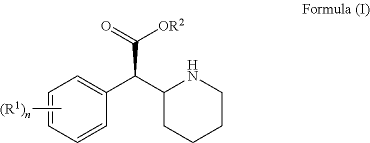
or pharmaceutically acceptable salts, solvates, or hydrates thereof, wherein
each R¹ is independently selected from hydrogen, halo, C₁₋₆alkyl, C₁₋₆haloalkyl, C₁₋₆ alkoxy, C₁₋₆haloalkoxy, —OR³, —C(O)R³, and —C(O)OR³;
R² is selected from hydrogen and C₁₋₆alkyl;
R³ is selected from hydrogen, C₁₋₆alkyl, C₃₋₆cycloalkyl, and C₆₋₁₀aryl; and
n is selected from 1, 2, 3, 4, and 5;
b. one or more compounds of Formula (II) 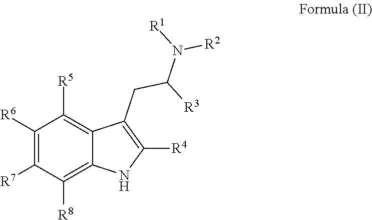
or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆-haloalkoxy, and —N(R⁹)₂;
R⁴, R⁵, R⁶, R⁷, and R⁸ are independently selected from hydrogen, halo, C₁₋₆alkyl, C₁₋₆ haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl; and
one or more compounds of Formula III) 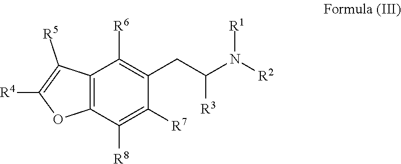
or pharmaceutically acceptable salts, solvates, or hydrates thereof, wherein
R¹ and R² are independently selected from C₁₋₆alkyl and C₂₋₆alkenyl;
R³ is selected from hydrogen, halo, C₁₋₆alkyl, C₁₋₆haloalkyl, C₁₋₆alkoxy, C₁₋₆haloalkoxy, and —N(R⁹)₂;
R⁴, R⁵, R⁶, R⁷, and R⁸ are independently selected from hydrogen, halo, C₁₋₆alkyl, C₁₋₆-haloalkyl, C₁₋₆alkoxy, C₁₋₆haloalkoxy, —N(R⁹)₂, —OR⁹, —OC(O)R⁹, —C(O)R⁹, and —C(O)OR⁹; and
R⁹ is selected from hydrogen, C₁₋₆alkyl, C₁₋₆haloalkyl C₃₋₆cycloalkyl, and C₆₋₁₀aryl.

71. The combination of claim 70, wherein said combination comprises about 1 mg to about 20 mg of said one or more compounds of Formula (I) or pharmaceutically acceptable salts, solvates, or hydrates thereof.

72. The combination of any one of the preceding claims, wherein said combination comprises about 1 mg to about 20 mg of said one or more compounds of Formula (II) or pharmaceutically acceptable salts, solvates, or hydrates thereof.

73. The combination of any one of the preceding claims, wherein said combination comprises about 1 mg to about 140 mg of said one or more compounds of Formula (III) or pharmaceutically acceptable salts, solvates, or hydrates thereof.

74. The combination of any one of the preceding claims, wherein said one or more compounds of Formula (III) are present at an amount that is 2-fold, 3-fold, 4-fold, 5-fold, 6-fold, 7-fold, or 8-fold greater than said amount of said one or more compounds of Formula (I), said one or more compounds of Formula (II), or both.

75. The combination of any one of the preceding claims, wherein said one or more compounds of Formula (III) are present at an amount that is 7-fold greater than said amount of said one or more compounds of Formula (I) and said one or more compounds of Formula (II).

76. The combination of any one of the preceding claims, comprising:
a. 10 mg of said one or more compounds of Formula (I);
b. 10 mg of said one or more compounds of Formula (II); and
c. 70 mg of said one or more compounds of Formula (III).

77. The combination of any one of the preceding claims, wherein said one or more compounds of Formula (I) is Compound 1.

78. The combination of any one of the preceding claims, wherein said one or more compounds of Formula (II) is Compound 2.

79. The combination of any one of the preceding claims, wherein said one or more compounds of Formula (III) is Compound 3.

80. A method for treating a disease, a disorder, or a condition in a subject in need thereof, the method comprising administering to said subject:
the combination of any one of claims 70-79, or pharmaceutically acceptable salts, solvates, or hydrates thereof.

81. The method of claim 80, wherein the disease, the disorder, or the condition is selected from sexual abuse, mood disorders, phobia disorders, dissociative disorders, stress disorders, personality disorders, obsessive compulsive disorders, tourette's disorder, grief, autism, eating disorders, alcohol use disorder, conduct disorder, conversion disorder, and addiction.

82. The method of claim 80, wherein the sexual abuse is selected from adult sexual disorder and child sexual disorder.

83. The method of claim 80, wherein the mood disorder is selected from persistent depressive disorder or dysthymia, major depressive disorder, cyclothymic disorder, premenstrual dysphoric disorder, generalized anxiety disorder, and panic disorder.

84. The method of claim 80, wherein the phobia disorder is selected from phobia of animal, phobia of blood-injection-injury, phobia of blood, phobia of injections or transfusions, phobia of injury, phobia of medical care, phobia of natural environment, and situational phobias.

85. The method of claim 80, wherein the stress disorder is selected from acute stress disorder, posttraumatic stress disorder, unspecified trauma- and stressor-related disorder, nightmare disorder, adjustment disorder, reactive attachment disorder, and disinhibited attached disorder.

86. The method of claim 80, wherein the personality disorder is selected from antisocial behavior, antisocial personality disorder, avoidant personality disorder, borderline personality disorder, narcissistic personality disorder, and obsessive-compulsive personality disorder.

87. The method of claim 80, wherein the eating disorder is selected from avoidant/restrictive food intake disorder, binge-eating disorder, body dysmorphic disorder, bulimia nervosa, and anorexia nervosa.

88. The method of claim 80, wherein the addiction is selected from tobacco use addiction, opioid use disorder, and alcohol use disorder.
